(19) United States
(12) Patent Application Publication
(10) Pub. No.: US 2023/0365519 A1
(43) Pub. Date: Nov. 16, 2023

(54) CARBOXYLIC DIARYLTHIAZEPINEAMINES AND USES THEREOF

(71) Applicant: Kures, Inc., New York, NY (US)

(72) Inventor: Andrew Kruegel, Secaucus, NJ (US)

(21) Appl. No.: 18/030,492

(22) PCT Filed: Oct. 6, 2021
(86) PCT No.: PCT/IB2021/059179

§ 371 Date: Apr. 6, 2023

Related U.S. Application Data
(60) Provisional application No. 63/088,421, filed on Oct. 6, 2020.

(51) Int. Cl. C07D 281/02; C07D 417/12; C07D 513/04

(52) U.S. Cl. CPC C07D 281/02; C07D 417/12; C07D 513/04

(57) ABSTRACT

The present disclosure provides a compound having the structure: Formula (I) wherein R₁ is —H or -(alkyl); R₂ is -(alkyl)-CO2H or -(alkyl)-CO2-(alkyl); R₃ is —H or -(alkyl); and X is —Br or —I, or a pharmaceutically acceptable salt or ester thereof, for treating or preventing a neurological disorder, including Huntington’s disease, Rett syndrome, and CDKL5 disorder.

All references, including patents and patent applications, cited in this specification are hereby incorporated by reference. No admission is made that any reference constitutes prior art. Nor does discussion of any reference constitute an admission that such reference forms part of the common general knowledge in the art, in any country.

### BACKGROUND OF THE INVENTION

The mu-opioid receptor (MOR) has been recognized as an important molecular target for several decades. However, the vast majority of MOR agonists used clinically today are structurally related to or derived from morphine (and other poppy alkaloids). These compounds suffer from many serious problems, including development of tolerance (increased dosing is required to achieve the same effects), high addiction liability, and other side effects (e.g., respiratory depression, nausea, and constipation) (Williams, J.T. et al. 2013). Therefore, there is a continuing interest in the development of new MOR agonists with improved therapeutic profile (Corbett, A.D. et al. 2006).

MOR agonists have been widely used for pain treatment. There is also historical and growing interest in the use of MOR agonists as medicaments for depression. Prior to the adoption of tricyclic antidepressants and electroshock therapy as favored treatments for depression, opiates were among the only options available, with the “opium cure” being an accepted treatment modality in the early 20th century (Berrocoso, E. et al. 2009). More recently, studies in both rodents (Besson, A. et al. 1996) and humans (Bodkin, J.A. et al. 1995) have suggested that MOR activation may lead to antidepressant and/or anxiolytic effects. The antidepressant tianeptine has also been reported to act as a full agonist of the MOR (Gassaway, M.M. et al. 2014).

On the molecular level, MORs are extensively expressed in the hippocampus and have been shown to exert a variety of indirect modulatory effects on glutamatergic neurons in this brain region (Xie, C.W. et al. 1997; Svoboda, K.R. et al. 1999). Normalization and modulation of glutamate signaling has been strongly associated with the actions of antidepressants (Paul, I.A. and Skolnick, P. 2003) and indeed, the NMDA antagonist ketamine, shows rapid and efficacious antidepressant activity in human clinical trials (Zarate, C.A. Jr et al. 2006). Further, agonists of the related delta-opioid receptor (DOR) have been demonstrated to show robust antidepressant efficacy (Jutkiewicz, E.M. 2006).

Opioid receptor dysfunction may also be associated with borderline personality disorder (BPD). Patients afflicted with BPD exhibit alterations in both basal MOR binding potential and endogenous opioid responses to negative stimuli (Prossin, A.R. et al. 2010). There is also a high prevalence of BPD among patients seeking buprenorphine treatment for opioid addiction (Sansone, R.A. et al. 2008). Accordingly, MOR modulators may be useful medicaments for BPD.

Long-acting prescription opioids may also be used as maintenance (replacement) therapies in the treatment of opioid addiction. In this case, a prescription opioid is provided to the patient chronically and under medical supervision to substitute for the use of illicit opioids (e.g., heroin), thus reducing cravings for, and abuse of, the illicit drug. Opioid maintenance therapy is considered a standard method of care for opioid addiction and is more successful than behavioral or antagonist interventions (Bart, G. 2012).

Unfortunately, respiratory depression is a major liability associated with the use of MOR agonists (Pattinson, K.T.S. 2008). This side effect may complicate medical use of opioids both in the control of pain and alongside other medications that also depress respiration. Similarly, the primary cause of death in cases of acute opioid overdose is respiratory failure. Accordingly, methods of controlling or reversing opioid-induced respiratory depression associated with opioid use or overdose are of high interest. Likewise, new opioid agents with reduced potential to cause respiratory depression are also valuable.

The standard of care for opioid-induced respiratory depression and overdose is administration of naloxone, an MOR antagonist (Wermeling, D.P. 2015). Although effective, naloxone treatment is associated with several important shortcomings. For one, naloxone also reverses the analgesic effects of MOR agonists and is thus inappropriate for use in settings where pain management is required (e.g., postoperatively). Naloxone administration also induces precipitated withdrawal symptoms, which although not typically life threatening, are extremely unpleasant for the patient. Lastly, naloxone is not orally bioavailable and exhibits a short half-life, meaning that it must be administered via parenteral routes and, when treating respiratory depression from long-acting opioids, continuously to overcome the short duration of action. Therefore, new therapeutic strategies to address opioid-induced respiratory depression or overdose without these shortcomings are desirable.

In addition to pain, anxiety, behavioral disorders, and mood disorders, there are other serious neurological disorders that afflict patients. Disorders such as Huntington’s disease, Rett syndrome, and CDKL5 disorder, amongst others, have significant deleterious effects, with limited or no treatment options available. Therefore, there is an ongoing need for therapeutics to address these neurological disorders and their symptoms.

### SUMMARY OF THE INVENTION

The present disclosure provides a compound having the structure:

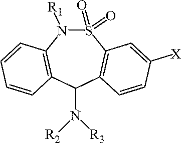

wherein
R₁ is —H or -(alkyl);
R₂ is -(alkyl)—CO₂H or -(alkyl)—CO₂—(alkyl);
R₃ is —H or -(alkyl); and
X is —Br or —I, or a pharmaceutically acceptable salt or ester thereof,
wherein the compound is for treating or preventing a neurological disorder in a subject, wherein the neurological disorder is selected from the group consisting of Huntington’s disease; Rett syndrome; a Rett syndrome variant; Angelman syndrome; Prader-Willi syndrome; neonatal onset encephalopathy; X-linked recessive mental retardation; fetal alcohol spectrum disorder; Hirschsprung disease; CDKL5 disorder; West syndrome; FOXG1 syndrome; and Lennox-Gastaut syndrome.

The present disclosure further provides a process for producing a compound having the structure:

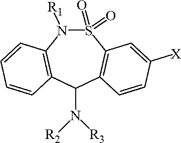

comprising
(a) contacting the compound having the structure:

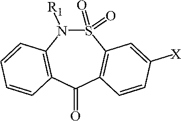

with a reducing agent in a first suitable solvent to produce a compound having the structure: 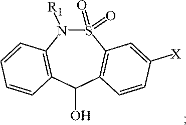
(b) reacting the product of step (a) with a halogenating agent, tosylating agent or triflating agent in a second suitable solvent so as to produce a compound having the structure:

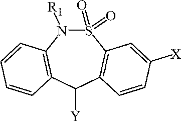

wherein Y is OTs, OTf, Cl, Br, or I;
(c) reacting the product of step (b) with an amine in the presence of a base in a third suitable solvent so as to produce the compound having the structure:

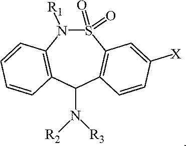

wherein the compound is for treating or preventing a neurological disorder in a subject, wherein the neurological disorder is selected from the group consisting of Huntington’s disease; Rett syndrome; a Rett syndrome variant; Angelman syndrome; Prader-Willi syndrome; neonatal onset encephalopathy; X-linked recessive mental retardation;
fetal alcohol spectrum disorder; Hirschsprung disease; CDKL5 disorder; West syndrome; FOXG1 syndrome; and Lennox-Gastaut syndrome.

The present disclosure also provides a method of treating a subject afflicted with a neurological disorder, wherein the neurological disorder is selected from the group consisting of Huntington’s disease; Rett syndrome; a Rett syndrome variant; Angelman syndrome; Prader-Willi syndrome; neonatal onset encephalopathy; X-linked recessive mental retardation; fetal alcohol spectrum disorder; Hirschsprung disease; CDKL5 disorder; West syndrome; FOXG1 syndrome; and Lennox-Gastaut syndrome comprising administering to the subject an effective amount of a DOR agonist or DOR antagonist and an effective amount of a compound having the structure:

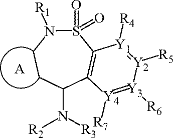

wherein
A is an aryl or heteroaryl, with or without substitution;
R₁ is —H or -(alkyl);
R₂ is -(alkyl), -(alkenyl), -(alkynyl), -(alkyl)—OH, -(alkyl)-CO₂H, -(alkyl)—CO₂-(alkyl), -(alkyl) —C(O)—NH₂, -(alkyl) -C(O)-NH(alkyl), -(alkyl)-C(O)-NH-(hydroxyalkyl), -(alkyl)—C(O)—N(alkyl)₂, -(alkyl)-C(O)-N(hydroxyalkyl)₂, -(alkyl)-O-(alkyl), -(alkyl)-S-(alkyl), -(alkyl)-CF3, -(alkyl)-O-(hydroxyalkyl), -(alkyl)-O-(alkyl)-O-(alkyl), -(alkyl)-(CH)-(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, -NH-(alkyl), -NH-(alkenyl), -NH-(alkynyl), -NH-(aryl), -NH-(heteroaryl), —OH, —OAc, -O-C(O)(alkyl), -O-(alkyl), -O-(alkylaryl), -O-(alkenyl), -O-(alkynyl), -O-(aryl), -O-(heteroaryl), -S-(alkyl), -S-(alkenyl), -S-(alkynyl), -S-(aryl), -S-(heteroaryl), -S(O)-(alkyl), -S(O)-(aryl), -S(O)-(heteroaryl), -SO₂-(alkyl), -SO₂(aryl), or -SO2-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
or a pharmaceutically acceptable salt thereof,
so as to thereby treat the neurological disorder.

The present disclosure provides a method of treating a subject afflicted with a neurological disorder, wherein the neurological disorder is selected from the group consisting of Huntington’s disease; Rett syndrome; a Rett syndrome variant; Angelman syndrome; Prader-Willi syndrome; neonatal onset encephalopathy; X-linked recessive mental retardation; fetal alcohol spectrum disorder; Hirschsprung disease; CDKL5 disorder; West syndrome; FOXG1 syndrome; and Lennox-Gastaut syndrome comprising administering to the subject an effective amount of a compound having the structure:

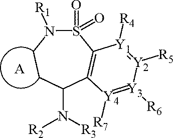

wherein
A is an aryl or heteroaryl, with or without substitution;
R₁ is —H or -(alkyl);
R₂ is -(alkyl), -(alkenyl), -(alkynyl), -(alkyl)—OH, -(alkyl)-CO₂H, -(alkyl)-CO₂-(alkyl), -(alkyl) —C(O)—NH₂, -(alkyl) -C(O)-NH(alkyl), -(alkyl)-C(O)-NH-(hydroxyalkyl), -(alkyl)-C(O)-N(alkyl)₂, -(alkyl)-C(O)-N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-O-(alkyl), -(alkyl)-(CH)-(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-
(1,3-dioxane) or -(alkyl)-(4,5-dihydrooxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, -NH-(alkyl), -NH-(alkenyl), -NH-(alkynyl), -NH-(aryl), -NH-(heteroaryl), —OH, —OAc, —O—C(O) (alkyl), -O-(alkyl), -O-(alkylaryl), -O-(alkenyl), -O-(alkynyl), -O-(aryl), -O-(heteroaryl), -S-(alkyl), -S-(alkenyl), -S-(alkynyl), -S-(aryl), -S-(heteroaryl), -S(O)-(alkyl), -S(O)-(aryl), -S(O)-(heteroaryl), —SO₂—(alkyl), —SO₂—(aryl), or -SO2-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
or a pharmaceutically acceptable salt thereof,
so as to thereby treat the neurological disorder.

The present disclosure also provides a method of treating a subject afflicted with a neurological disorder, wherein the neurological disorder is selected from the group consisting of Huntington’s disease; Rett syndrome; a Rett syndrome variant; Angelman syndrome; Prader-Willi syndrome; neonatal onset encephalopathy; X-linked recessive mental retardation; fetal alcohol spectrum disorder; Hirschsprung disease; CDKL5 disorder; West syndrome; FOXG1 syndrome; and Lennox-Gastaut syndrome comprising administering to the subject an effective amount of an NMDA receptor antagonist, an NMDA receptor partial agonist, a neurokinin 1 receptor antagonist, a DOR agonist or a DOR antagonist and an effective amount of a compound having the structure:

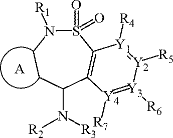

wherein
A is an aryl or heteroaryl, with or without substitution;
R₁ is —H or -(alkyl);
R₂ is -(alkyl), -(alkenyl), -(alkynyl), -(alkyl)-OH, -(alkyl)-CO₂H, -(alkyl)-CO₂-(alkyl), -(alkyl) —C(O)—NH₂, -(alkyl) -C(O)-NH(alkyl), -(alkyl)-C(O)-NH-(hydroxyalkyl), -(alkyl)-C(O)-N(alkyl)₂, -(alkyl)-C(O)-N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-O-(alkyl), -(alkyl)-(CH)-(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, -NH-(alkyl), -NH-(alkenyl), -NH-(alkynyl), -NH-(aryl), -NH-(heteroaryl), —OH, —OAc, —O—C(O) (alkyl), -O-(alkyl), -O-(alkylaryl), -O-(alkenyl), -O-(alkynyl), -O-(aryl), -O-(heteroaryl), -S-(alkyl), -S-(alkenyl), -S-(alkynyl), -S-(aryl), -S-(heteroaryl), -S(O)-(alkyl), -S(O)-(aryl), -S(O)-(heteroaryl), -SO₂-(alkyl), -SO₂-(aryl), or -SO2-(heteroaryl); and
Y₁, Y₂, Y₃ and Y₄ are each independently N or C,
   wherein when Y₃ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
or a pharmaceutically acceptable salt thereof,
so as to thereby treat the neurological disorder.

The present disclosure further provides a method of treating a subject afflicted with a neurological disorder, wherein the neurological disorder is selected from the group consisting of Huntington’s disease; Rett syndrome; a Rett syndrome variant; Angelman syndrome; Prader-Willi syndrome; neonatal onset encephalopathy; X-linked recessive mental retardation; fetal alcohol spectrum disorder; Hirschsprung disease; CDKL5 disorder; West syndrome; FOXG1 syndrome; and Lennox-Gastaut syndrome comprising administering to the subject an effective amount of naloxone or methylnaltrexone and an effective amount of a compound having the structure:

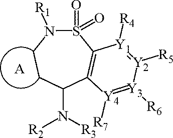

wherein
A is an aryl or heteroaryl, with or without substitution;
R₁ is —H or -(alkyl);
R₂ is -(alkyl), -(alkenyl), -(alkynyl), -(alkyl)-OH, -(alkyl)-CO₂H, -(alkyl)-CO₂-(alkyl), -(alkyl) —C(O)—NH₂, -(alkyl) -C(O)-NH(alkyl), -(alkyl)-C(O)-NH-(hydroxyalkyl), -(alkyl)-C(O)-N(alkyl)₂, -(alkyl)-C(O)-N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-O-(alkyl), -(alkyl)-(CH)-(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, -NH-(alkyl), -NH-(alkenyl), -NH-(alkynyl), -NH-(aryl), -NH-(heteroaryl), —OH, —OAc, -O-C(O) (alkyl), -O-(alkyl), -O-(alkylaryl), -O-(alkenyl), -O-(alkynyl), -O-(aryl), -O-(heteroaryl), -S-(alkyl), -S-(alkenyl), -S-(alkynyl), -S-(aryl), -S-(heteroaryl), -S(O)-(alkyl), -S(O)-(aryl), -S(O)-(heteroaryl), -SO₂-(alkyl), -SO₂-(aryl), or -SO2-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
or a pharmaceutically acceptable salt thereof,
so as to thereby treat the neurological disorder.

It should also be appreciated that compounds as defined herein, or compositions as defined herein can be used for the manufacture of a medicament for treating a subject afflicted with a neurological disorder, wherein the neurological disorder is selected from the group consisting of Huntington’s disease; Rett syndrome; a Rett syndrome variant; Angelman syndrome; Prader-Willi syndrome; neonatal onset encephalopathy; X-linked recessive mental retardation; fetal alcohol spectrum disorder; Hirschsprung disease; CDKL5 disorder; West syndrome; FOXG1 syndrome; and Lennox-Gastaut syndrome.

The foregoing brief summary broadly describes certain features and technical advantages. Further features and technical advantages will be described in the detailed description and examples that follow.

Other features and advantages will be better understood from the detailed description read in connection with the accompanying examples. However, the examples provided herein are intended to help illustrate the invention or assist with developing an understanding of the invention, and are not intended to limit the invention’s scope.

### BRIEF DESCRIPTION OF THE FIGURES

FIG. 1A: DSBA data for compound 84. Odds ratios that the behavioral phenotypes induced by drug treatments are similar to the inverse of the behavioral phenotype exhibited by Q175 heterozygous mice at 6 months of age. An odds ratio of 1 suggests a random relationship. *p < 0.05; **p < 0.01.

FIG. 1B: DBSA data for compound 7e. Odds ratios that the behavioral phenotypes induced by drug treatments are similar to the inverse of the behavioral phenotype exhibited by Q175 heterozygous mice at 6 months of age. An odds ratio of 1 suggests a random relationship. *p < 0.05; **p < 0.01.

FIG. 2A: DSBA data for compound 84. Odds ratios that the behavioral phenotypes induced by drug treatments are similar to the inverse of the behavioral phenotype exhibited by Q175 heterozygous mice at 10 months of age. An odds ratio of 1 suggests a random relationship. *p < 0.05; **p < 0.01.

FIG. 2B: DSBA data for compound 7e. Odds ratios that the behavioral phenotypes induced by drug treatments are similar to the inverse of the behavioral phenotype exhibited by Q175 heterozygous mice at 10 months of age. An odds ratio of 1 suggests a random relationship. *p < 0.05; **p < 0.01.

### DETAILED DESCRIPTION OF THE INVENTION

The following description sets forth numerous exemplary configurations, parameters, and the like. It should be recognized, however, that such description is not intended as a limitation on the scope of the present disclosure, but is instead provided as a description of exemplary embodiments.

In each instance herein, in descriptions, embodiments, and examples of the present disclosure, the terms “comprising”, “including”, etc., are to be read expansively, without limitation. Thus, unless the context clearly requires otherwise, throughout the description and the claims, the words “comprise”, “comprising”, and the like are to be construed in an inclusive sense as to opposed to an exclusive sense, that is to say in the sense of “including but not limited to”.

The term “consisting essentially of”, as used herein, may refer to the presence of a component in a composition. For example, a concentrate may be at least 80% by weight of the composition, or at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.8%, or at least 99.9% by weight of the composition (% w/w). For liquids, a concentrate may be at least 80% by volume of the composition volume, or at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.8%, or at least 99.9% by volume of the composition volume (% v/v).

In the present description, the articles “a” and “an” are used to refer to one or to more than one (i.e., to at least one) of the grammatical object of the article. By way of example, “an element” can be taken to mean one element or more than one element.

Throughout this description, the term “approximately” is used to indicate that a value includes the standard deviation of error for the method being employed to determine the value, for example, dosage levels, as described in detail herein. In particular, the term “approximately” encompasses a 10% to 15% deviation (positive and negative) in the stated value or range, particularly 10% deviation (positive and negative) in the stated value or range.

“Neurological disorder” refers to various conditions of the neurological system including neurodegenerative and neurodevelopmental conditions. Specifically included are Huntington’s disease; Rett syndrome; Rett syndrome variants; Angelman syndrome; Prader-Willi syndrome; neonatal onset encephalopathy; X-linked recessive mental retardation; fetal alcohol spectrum disorder; Hirschsprung disease; CDKL5 disorder; West syndrome; FOXG1 syndrome; and Lennox-Gastaut syndrome.

“Symptoms” of a neurological disorder refer to various physical effects exhibited in subjects. Physical symptoms can include one or more of: involuntary movements (e.g., chorea); abnormal muscle rigidity or contraction (e.g., dystonia); abnormal hand movements (e.g., stereotypies); poor muscle tone (e.g., hypotonia), unusual eye movements; unusual facial expressions; tremors; impaired gait, posture, or balance; frequent falls; loss of balance; difficulty with swallowing; teeth grinding (e.g., bruxism); fatigue; and insomnia. Cognitive symptoms are also noted, and may include one or more of: difficulties with concentration or organization; communication difficulties; perseveration; lack of impulse control; lack of self-awareness; slowed thought processing; and learning difficulties. Serious symptoms are noted, including breathing problems; irregular heartbeat; and seizures.

“Treating” as used herein refers to reducing, ameliorating, or resolving a disorder, for example a neurological disorder, such as Huntington’s disease, Rett syndrome, or CDKL5 disorder. A treatment is expected to result in the reduction, amelioration, or elimination of one or more symptoms of the disorder.

“Preventing” as used herein refers stopping or delaying the onset of a disorder, for example a neurological disorder, such as Huntington’s disease, Rett syndrome, or CDKL5 disorder. A preventative measure is expected to result in the inhibition or delay in onset of one or more symptoms of the disorder, the lessening of symptoms if such do arise, and/or the inhibition or delay of the progression of the disorder.

It should be understood that the term “treating or preventing” does not exclude the possibility of obtaining both treatment and prevention (e.g., at the same time or at different times) of a disorder in any given subject.

Of particular interest in the present disclosure is the use of a compound of this disclosure in the treatment or prevention of one or more neurological disorders in a subject. This includes neurodegenerative diseases and neurodevelopmental disorders, for example, Huntington’s disease; Rett syndrome which includes Rett syndrome variants, such as the Rett syndrome Rolando variant (congenital variant), the Rett syndrome Zappella variant, the Rett syndrome Hanefeld variant (early epilepsy variant); Angelman syndrome; Prader-Willi syndrome; neonatal onset encephalopathy; X-linked recessive mental retardation (MRX); fetal alcohol spectrum disorder; Hirschsprung disease; CDKL5 disorder; West syndrome (infantile spasms, ISSX); FOXG1 syndrome; and Lennox-Gastaut syndrome.

Notably, the Huntingtin gene product (Htt), is known to interact with the gene product associated with Rett syndrome (MeCP2). The MeCP2 gene product is also associated with Angelman syndrome, Prader-Willi syndrome, neonatal onset encephalopathy, X-linked recessive mental retardation, fetal alcohol spectrum disorder, and Hirschsprung disease. CDKL5 and FOXG1 mutations may be associated with Rett syndrome, and CDKL5 and FOXG1 mutations lead to symptoms that overlap with Rett syndrome symptoms. Accordingly, CDKL5 and FOXG1 disorders have been referred to as Rett syndrome related disorders.

The present disclosure provides a compound having the structure:

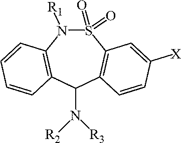

wherein
R₁ is —H or -(alkyl);
R₂ is —(alkyl)—CO₂H or —(alkyl)—CO₂—(alkyl);
R₃ is —H or -(alkyl); and
X is —Br or —I,
or a pharmaceutically acceptable salt or ester thereof,
wherein the compound is for treating or preventing a neurological disorder, wherein the neurological disorder is selected from the group consisting of Huntington’s disease, Rett syndrome, and CDKL5 disorder, and the other neurological disorders as described herein.

In some embodiments, wherein
R₁ is —H or -(alkyl);
R₂ is -(C₂-C₅ alkyl)-CO₂H or -(C₂-C₅ alkyl)-CO₂-(alkyl);
R₃ is —H or -(alkyl); and
X is —Br or —I.

In some embodiments, wherein
R₁ is —H or -(alkyl);
R₂ is -(C₂-C₅ alkyl)-CO₂H;
R₃ is —H or -(alkyl); and
X is —Br or —I.

In some embodiments, wherein
R₁ is —H or -(alkyl);
R₂ is -(C₂-C₅ alkyl)-CO₂-(alkyl);
R₃ is —H or -(alkyl); and
X is —Br or —I.

In some embodiments, wherein
R₁ is —H or -(alkyl);
R₂ is -(C₂-C₅ alkyl) —CO₂CH₃;
R₃ is —H or -(alkyl); and
X is —Br or —I.

In some embodiments, wherein
R₁ is —H or -(alkyl);
R₂ is -(C₂-C₅ alkyl)-CO₂CH₂CH₃;
R₃ is —H or -(alkyl); and
X is —Br or —I.

In some embodiments, wherein R₁ is —H, —CH₃ or —CH₂CH₃.

In some embodiments, wherein R₃ is —H, —CH₃ or —CH₂CH₃.

In some embodiments, wherein X is —Br.

In some embodiments, wherein X is —I.

In some embodiments, wherein
R₂ is —(C₂ alkyl) —CO₂H, —(C₃ alkyl) —CO₂H, —(C₄ alkyl) —CO₂H, or (C₅
alkyl) —CO₂H; and
R₃ is —H.

In some embodiments, wherein
R₂ is —(C₂ alkyl) —CO₂CH₃, —(C₃ alkyl) —CO₂CH₃, —(C₄ alkyl) —CO₂CH₃, or
—(C₅ alkyl) —CO₂CH₃; and
R₃ is —H.

In some embodiments, wherein
R₂ is -(C₂ alkyl)-CO₂CH₂CH₃, -(C₃ alkyl)-CO₂CH₂CH₃, —(C₄ alkyl)-
CO₂CH₂CH₃, or -(C₅ alkyl)-CO₂CH₂CH₃; and
R₃ is —H.

In some embodiments, wherein
R₁ is -(alkyl);
R₂ is -(alkyl)-CO₂H or -(alkyl)-CO₂-(alkyl);
R₃ is —H; and
X is —Br or —I.

In some embodiments, wherein wherein
R₁ is -(alkyl);
R₂ is -(C₂-C₅ alkyl)-CO₂H or -(C₂-C₅ alkyl) —CO₂CH₃;
R₃ is —H; and
X is —Br or I—,
or a pharmaceutically acceptable salt or ester thereof.

In some embodiments, wherein wherein
R₁ is -(alkyl);
R₂ is -(C₂-C₅ alkyl)-CO₂H or -(C₂-C₅ alkyl) —CO₂CH₂CH₃
R₃ is —H; and
X is —Br or I—,
or a pharmaceutically acceptable salt or ester thereof.

In some embodiments, the compound having the structure:

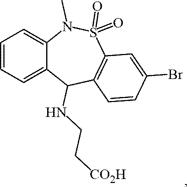

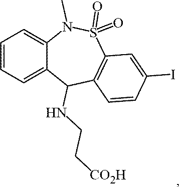

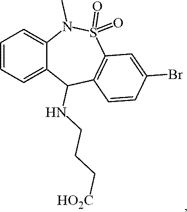

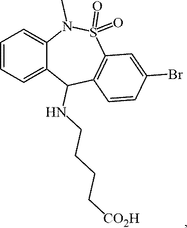

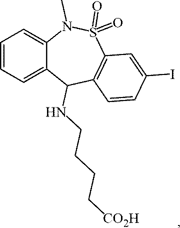

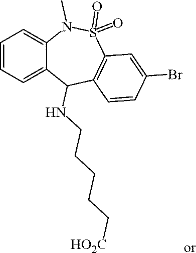

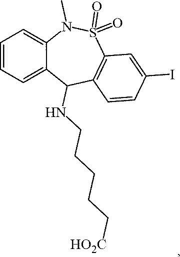

or a pharmaceutically acceptable salt or ester thereof.

In some embodiments, the compound having the structure:

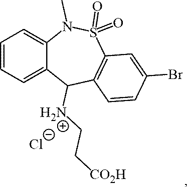

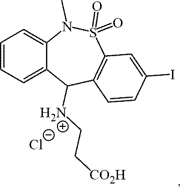

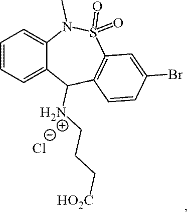

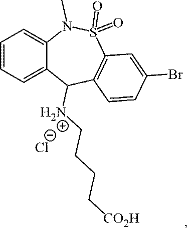

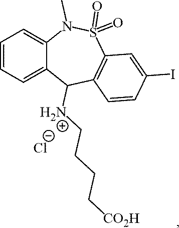

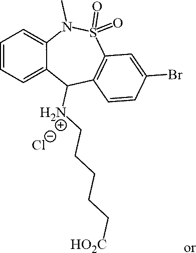

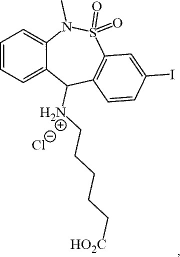

or a pharmaceutically acceptable salt or ester thereof.

In some embodiments, the compound having the structure:

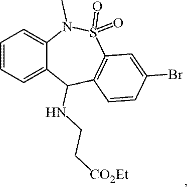

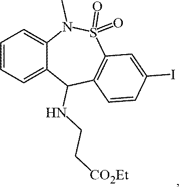

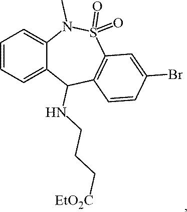

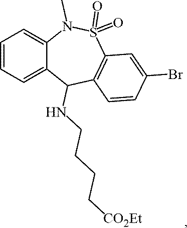

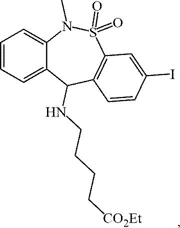

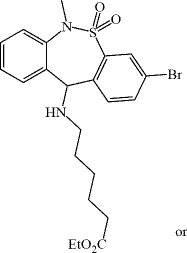

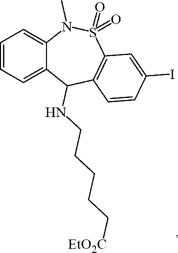

or a pharmaceutically acceptable salt or ester thereof.

In some embodiments, a pharmaceutical composition comprising a compound of the present disclosure and a pharmaceutically acceptable carrier is for treating or preventing a neurological disorder, wherein the neurological disorder is selected from the group consisting of Huntington’s disease, Rett syndrome, and CDKL5 disorder, and the other neurological disorders as described herein.

In some embodiments, a method of activating a mu-opioid receptor or delta-opioid receptor comprising contacting the mu-opioid receptor or delta-opioid receptor with a compound of the present disclosure, to thereby treat a neurological disorder, wherein the neurological disorder is selected from the group consisting of Huntington’s disease, Rett syndrome, and CDKL5 disorder, and the other neurological disorders as described herein.

In some embodiments, a method of treating a subject afflicted with a neurological disorder, wherein the neurological disorder is selected from the group consisting of Huntington’s disease, Rett syndrome, and CDKL5 disorder, and the other neurological disorders as described herein, comprising administering an effective amount of a compound of the present disclosure to the subject so as to treat the neurological disorder.

In some embodiments, a method of treating a subject afflicted with a neurological disorder, wherein the neurological disorder is Huntington’s disease comprising administering an effective amount of a compound of the present disclosure to the subject so as to treat the neurological disorder.

In some embodiments, a method of treating a subject afflicted with a neurological disorder, wherein the neurological disorder is Rett syndrome comprising administering an effective amount of a compound of the present disclosure to the subject so as to treat the neurological disorder.

In some embodiments, a method of treating a subject afflicted with a neurological disorder, wherein the neurological disorder is CDKL5 disorder comprising administering an effective amount of a compound of the present disclosure to the subject so as to treat the neurological disorder.

In some embodiments, a method of treating a subject afflicted with a neurological disorder, wherein the neurological disorder is selected from the group consisting of Huntington’s disease, Rett syndrome, and CDKL5 disorder, and the other neurological disorders as described herein comprising administering to the subject an effective amount of an NMDA receptor antagonist, an NMDA receptor partial agonist, a neurokinin 1 receptor antagonist or a DOR agonist and an effective amount of a compound of the present disclosure so as to thereby treat the neurological disorder.

In some embodiments, a method of treating a subject afflicted with a neurological disorder, wherein the neurological disorder is selected from the group consisting of Huntington’s disease, Rett syndrome, and CDKL5 disorder, and the other neurological disorders as described herein comprising administering to the subject an effective amount of DOR antagonist and an effective amount of a compound of the present disclosure so as to thereby treat the neurological disorder.

In some embodiments, a method of treating a subject afflicted with a neurological disorder, wherein the neurological disorder is selected from the group consisting of Huntington’s disease, Rett syndrome, and CDKL5 disorder, and the other neurological disorders as described herein comprising administering to the subject an effective amount of an NMDA receptor antagonist, an NMDA receptor partial agonist, a neurokinin 1 receptor antagonist, a neurokinin 2 receptor antagonist, a neurokinin 3 receptor antagonist or a DOR agonist and an effective amount of a compound of the present disclosure so as to thereby treat the neurological disorder.

In some embodiments, a method of treating a subject afflicted with a neurological disorder, wherein the neurological disorder is selected from the group consisting of Huntington’s disease, Rett syndrome, and CDKL5 disorder, and the other neurological disorders as described herein comprising administering to the subject an effective amount of DOR antagonist and an effective amount of a compound of the present disclosure so as to thereby treat the neurological disorder.

In some embodiments, a method of treating a subject afflicted with a neurological disorder, wherein the neurological disorder is Huntington’s disease comprising administering to the subject an effective amount of an NMDA receptor antagonist, an NMDA receptor partial agonist, a neurokinin 1 receptor antagonist, a neurokinin 2 receptor antagonist, a neurokinin 3 receptor antagonist or a DOR agonist and an effective amount of a compound of the present disclosure so as to thereby treat the neurological disorder.

In some embodiments, a method of treating a subject afflicted with a neurological disorder, wherein the neurological disorder is Rett syndrome comprising administering to the subject an effective amount of an NMDA receptor antagonist, an NMDA receptor partial agonist, a neurokinin 1 receptor antagonist, a neurokinin 2 receptor antagonist, a neurokinin 3 receptor antagonist or a DOR agonist and an effective amount of a compound of the present disclosure so as to thereby treat the neurological disorder.

In some embodiments, a method of treating a subject afflicted with a neurological disorder, wherein the neurological disorder is CDKL5 disorder comprising administering to the subject an effective amount of an NMDA receptor antagonist, an NMDA receptor partial agonist, a neurokinin 1 receptor antagonist, a neurokinin 2 receptor antagonist, a neurokinin 3 receptor antagonist or a DOR agonist and an effective amount of a compound of the present disclosure so as to thereby treat the neurological disorder.

In some embodiments, a method of treating a subject afflicted with a neurological disorder, wherein the neurological disorder is Huntington’s disease comprising administering to the subject an effective amount of DOR antagonist and an effective amount of a compound of the present disclosure so as to thereby treat the neurological disorder.

In some embodiments, a method of treating a subject afflicted with a neurological disorder, wherein the neurological disorder is Rett syndrome comprising administering to the subject an effective amount of DOR antagonist and an effective amount of a compound of the present disclosure so as to thereby treat the neurological disorder.

In some embodiments, a method of treating a subject afflicted with a neurological disorder, wherein the neurological disorder is CDKL5 disorder comprising administering to the subject an effective amount of DOR antagonist and an effective amount of a compound of the present disclosure so as to thereby treat the neurological disorder.

In some embodiments, a method of treating a subject afflicted with a neurological disorder, wherein the neurological disorder is selected from the group consisting of Huntington’s disease, Rett syndrome, and CDKL5 disorder, and the other neurological disorders as described herein comprising administering to the subject an effective amount of an NMDA receptor antagonist, an NMDA receptor partial agonist, a neurokinin 1 receptor antagonist, a DOR agonist or a DOR antagonist and an effective amount of a pharmaceutical composition comprising a compound of the present disclosure or a salt or ester thereof, so as to thereby treat the neurological disorder.

In some embodiments, a method of treating a subject afflicted with a neurological disorder, wherein the neurological disorder is Huntington’s disease comprising administering to the subject an effective amount of an NMDA receptor antagonist, an NMDA receptor partial agonist, a neurokinin 1 receptor antagonist or a DOR antagonist and an effective amount of a pharmaceutical composition comprising a compound of the present disclosure or a salt or ester thereof, so as to thereby treat the neurological disorder.

In some embodiments, a method of treating a subject afflicted with a neurological disorder, wherein the neurological disorder is Rett syndrome comprising administering to the subject an effective amount of an NMDA receptor antagonist, an NMDA receptor partial agonist, a neurokinin 1 receptor antagonist or a DOR antagonist and an effective amount of a pharmaceutical composition comprising a compound of the present disclosure or a salt or ester thereof, so as to thereby treat the neurological disorder.

In some embodiments, a method of treating a subject afflicted with a neurological disorder, wherein the neurological disorder is CDKL5 disorder comprising administering to the subject an effective amount of an NMDA receptor antagonist, an NMDA receptor partial agonist, a neurokinin 1 receptor antagonist or a DOR antagonist and an effective amount of a pharmaceutical composition comprising a compound of the present disclosure or a salt or ester thereof, so as to thereby treat the neurological disorder.

In some embodiments, a method of treating a subject afflicted with a neurological disorder, wherein the neurological disorder is selected from the group consisting of Huntington’s disease, Rett syndrome, and CDKL5 disorder, and the other neurological disorders as described herein comprising administering to the subject an effective amount of a DOR agonist or a DOR antagonist and an effective amount of a pharmaceutical composition comprising a compound of the present disclosure or a salt or ester thereof, so as to thereby treat the neurological disorder.

In some embodiments, a method of treating a subject afflicted with a neurological disorder, wherein the neurological disorder is Huntington’s disease comprising administering to the subject an effective amount of a DOR agonist or a DOR antagonist and an effective amount of a pharmaceutical composition comprising a compound of the present disclosure or a salt or ester thereof, so as to thereby treat the neurological disorder.

In some embodiments, a method of treating a subject afflicted with a neurological disorder, wherein the neurological disorder is Rett syndrome comprising administering to the subject an effective amount of a DOR agonist or a DOR antagonist and an effective amount of a pharmaceutical composition comprising a compound of the present disclosure or a salt or ester thereof, so as to thereby treat the neurological disorder.

In some embodiments, a method of treating a subject afflicted with a neurological disorder, wherein the neurological disorder is CDKL5 disorder comprising administering to the subject an effective amount of a DOR agonist or a DOR antagonist and an effective amount of a pharmaceutical composition comprising a compound of the present disclosure or a salt or ester thereof, so as to thereby treat the neurological disorder.

The present disclosure further provides a process for producing a compound to be used for the methods described herein, the compound having the structure:

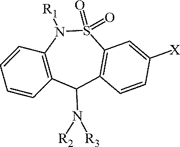

and the process comprising
(a) contacting the compound having the structure:

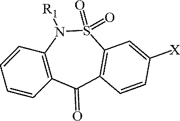

with a reducing agent in a first suitable solvent to produce a compound having the structure:

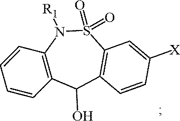

(b) reacting the product of step (a) with a halogenating agent or triflating agent in a second suitable solvent so as to produce a compound having the structure:

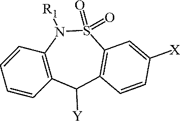

wherein Y is OTf, Cl, Br, or I;
(c) reacting the product of step (b) with an amine in the presence of a base in a third suitable solvent so as to produce the compound having the structure:

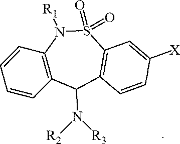

In some embodiments, the compound having the structure:

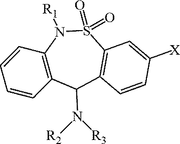

is prepared by a process comprising
(a) contacting the compound having the structure:

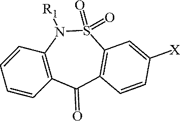

with a reducing agent in a first suitable solvent to produce a compound having the structure: 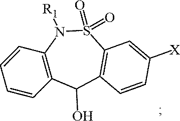
(b) reacting the product of step (a) with a halogenating agent, tosylating or triflating agent in a second suitable solvent so as to produce a compound having the structure:

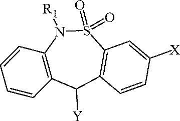

wherein Y is OTs, OTf, Cl, Br, or I;
(c) reacting the product of step (b) with an amine in the presence of a base in a third suitable solvent so as to produce the compound having the structure:

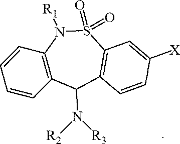

In some embodiments, the process wherein the reducing agent is sodium borohydride.

In some embodiments, the process wherein the halogenating agent is sulfonyl chloride or hydrogen chloride.

In some embodiments, the process wherein the halogenating agent is thionyl chloride or hydrogen chloride.

In some embodiments, the process wherein the amine is a primary amine or a secondary amine.

In some embodiments, the process wherein the first suitable solvent is methanol.

In some embodiments, the process wherein the second suitable solvent is dichloromethane.

In some embodiments, the process wherein the third suitable solvent is nitromethane.

The present disclosure yet further provides a method of treating a subject afflicted with a neurological disorder, wherein the neurological disorder is selected from the group consisting of Huntington’s disease, Rett syndrome, and CDKL5 disorder, and the other neurological disorders as described herein comprising administering to the subject an effective amount of a DOR agonist and an effective amount of a compound having the structure:

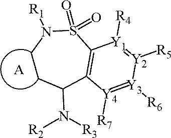

wherein
A is an aryl or heteroaryl, with or without substitution;
R₁ is —H or -(alkyl);
R₂ is -(alkyl), -(alkenyl), -(alkynyl), -(alkyl)-OH, -(alkyl)-CO₂H, -(alkyl)-CO₂-(alkyl), -(alkyl) —C(O)—NH₂, -(alkyl) —C(O)—NH(alkyl), -(alkyl)-C(O)-NH-(hydroxyalkyl), -(alkyl)-C(O)-N(alkyl)₂, -(alkyl)-C(O)-N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-O-(alkyl), -(alkyl)-(CH)-(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, -NH-(alkyl), -NH-(alkenyl), -NH-(alkynyl), -NH-(aryl), -NH-(heteroaryl), —OH, —OAc, -O-C(O) (alkyl), -O-(alkyl), -O-(alkylaryl), -O-(alkenyl), -O-(alkynyl), -O-(aryl), -O-(heteroaryl), -S-(alkyl), —S—(alkenyl), -S-(alkynyl), -S-(aryl), -S-(heteroaryl), -S(O)-(alkyl), -S(O)-(aryl), -S (O)- (heteroaryl), -SO₂-(alkyl), -SO₂-(aryl), or -SO2-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
or a pharmaceutically acceptable salt thereof,
so as to thereby treat the neurological disorder.

The present disclosure yet further provides a method of treating a subject afflicted with a neurological disorder, wherein the neurological disorder is selected from the group consisting of Huntington’s disease, Rett syndrome, and CDKL5 disorder, and the other neurological disorders as described herein comprising administering to the subject an effective amount of a compound having the structure:

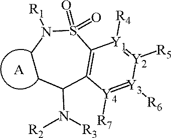

wherein
A is an aryl or heteroaryl, with or without substitution;
R₁ is —H or -(alkyl);
R₂ is -(alkyl), -(alkenyl), -(alkynyl), -(alkyl)-OH, -(alkyl)-CO₂H, -(alkyl)-CO₂-(alkyl), -(alkyl) —C(O)—NH₂, -(alkyl) -C(O)-NH(alkyl), -(alkyl)-C(O)-NH-(hydroxyalkyl), -(alkyl)-C(O)-N(alkyl)₂, -(alkyl)-C(O)-N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-O-(alkyl), -(alkyl)-(CH)-(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole); R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, -NH-(alkyl), -NH-(alkenyl), -NH-(alkynyl), -NH-(aryl), -NH-(heteroaryl), —OH, —OAc, -O-C(O)(alkyl), -O-(alkyl), -O-(alkylaryl), -O-(alkenyl), -O-(alkynyl), -O-(aryl), -O-(heteroaryl), -S-(alkyl), -S-(alkenyl), -S-(alkynyl), -S-(aryl), -S-(heteroaryl), -S(O)-(alkyl), -S(O)-(aryl), -S(O)-(heteroaryl), -SO₂-(alkyl), -SO₂-(aryl), or -SO2-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
or a pharmaceutically acceptable salt thereof,
so as to thereby treat the neurological disorder.

The present disclosure yet further provides a method of treating a subject afflicted with a neurological disorder, wherein the neurological disorder is selected from the group consisting of Huntington’s disease, Rett syndrome, and CDKL5 disorder, and the other neurological disorders as described herein comprising administering to the subject an effective amount of an NMDA receptor antagonist, an NMDA receptor partial agonist, a neurokinin 1 receptor antagonist, a DOR agonist or a DOR antagonist and an effective amount of a compound having the structure:

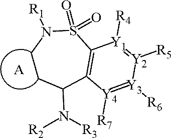

wherein
A is an aryl or heteroaryl, with or without substitution;
R₁ is —H or -(alkyl);
R₂ is -(alkyl), -(alkenyl), -(alkynyl), -(alkyl)-OH, -(alkyl)-CO₂H, -(alkyl)-CO₂-(alkyl), -(alkyl) —C(O)—NH₂, -(alkyl) -C(O)-NH(alkyl), -(alkyl)-C(O)-NH-(hydroxyalkyl), -(alkyl)-C(O)-N(alkyl)₂, -(alkyl)-C(O)-N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-O-(alkyl), -(alkyl)-(CH)-(O-(alkyl))₂, -(alkyl)-(heterocyclyl), -(alkyl)-OAc, -(alkyl)-tetrahydrofuran,
-(alkyl)-pyrrolidine, -(alkyl)-N-methylpyrrolidine, -(alkyl)-(1,3-dioxane) or -(alkyl)-(4,5-dihydrooxazole);
R₃ is —H or -(alkyl);
R₄, R₅, R₆ and R₇ are each absent or present, and when present, are each independently —H, —Cl, —Br, —F, —I, —CN, —CF₃, -OCF₃, -(alkyl), -(alkenyl), -(alkynyl), -(aryl), —NH₂, -NH-(alkyl), -NH-(alkenyl), -NH-(alkynyl), -NH-(aryl), -NH-(heteroaryl), —OH, —OAc, —O—C(O) (alkyl), -O-(alkyl), -O-(alkylaryl), -O-(alkenyl), -O-(alkynyl), -O-(aryl), -O-(heteroaryl), -S-(alkyl), —S—(alkenyl), -S-(alkynyl), -S-(aryl), -S-(heteroaryl), —S(O)—(alkyl), -S(O)-(aryl), -S(O)-(heteroaryl), -SO₂-(alkyl), -SO₂-(aryl), or -SO2-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
or a pharmaceutically acceptable salt thereof,
so as to thereby treat the neurological disorder.

The present disclosure yet further provides a method of treating a subject afflicted with a neurological disorder, wherein the neurological disorder is selected from the group consisting of Huntington’s disease, Rett syndrome, and CDKL5 disorder, and the other neurological disorders as described herein comprising administering to the subject an effective amount of a DOR antagonist and an effective amount of a compound having the structure:

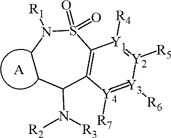

wherein
A is an aryl or heteroaryl, with or without substitution;
R₁ is —H or -(alkyl);
R₂ is -(alkyl), -(alkenyl), -(alkynyl), -(alkyl)-OH, -(alkyl)-CO₂H, -(alkyl)-CO₂-(alkyl), -(alkyl) —C(O)—NH₂, -(alkyl) -C(O)-NH(alkyl), -(alkyl)-C(O)-NH-(hydroxyalkyl), -(alkyl)-C(O)-N(alkyl)₂, -(alkyl)-C(O)-N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-O-(alkyl), -(alkyl)-(CH)-(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, -NH-(alkyl), -NH-(alkenyl), -NH-(alkynyl), -NH-(aryl), -NH-(heteroaryl), —OH, —OAc, —O—C(O) (alkyl), -O-(alkyl), -O-(alkylaryl), -O-(alkenyl), -O-(alkynyl), -O-(aryl), -O-(heteroaryl), -S-(alkyl), -S-(alkenyl), -S-(alkynyl), -S-(aryl), -S-(heteroaryl), -S(O)-(alkyl), -S(O)-(aryl), -S (O) - (heteroaryl), -SO₂-(alkyl), -SO₂-(aryl), or -SO2-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
or a pharmaceutically acceptable salt thereof,
so as to thereby treat the neurological disorder.

The present disclosure yet further provides a method of treating a subject afflicted with a neurological disorder, wherein the neurological disorder is Huntington’s disease comprising administering to the subject an effective amount of an NMDA receptor antagonist, an NMDA receptor partial agonist, a neurokinin 1 receptor antagonist or a DOR antagonist and an effective amount of a compound having the structure:

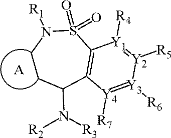

wherein
A is an aryl or heteroaryl, with or without substitution;
R₁ is —H or -(alkyl);
R₂ is -(alkyl), -(alkenyl), -(alkynyl), -(alkyl)-OH, -(alkyl)-CO₂H, -(alkyl)-CO₂-(alkyl), - (alkyl) —C(O) —NH₂, - (alkyl) -C(O) -NH(alkyl), -(alkyl)-C(O)-NH-(hydroxyalkyl), -(alkyl)-C(O)-N(alkyl)₂, -(alkyl)-C(O)-N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-O-(alkyl), -(alkyl)-(CH)-(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, -NH-(alkyl), -NH-(alkenyl), -NH-(alkynyl), -NH-(aryl), -NH-(heteroaryl), —OH, —OAc, —O—C(O) (alkyl), -O-(alkyl), -O-(alkylaryl), -O-(alkenyl), -O-(alkynyl), -O-(aryl), -O-(heteroaryl), -S-(alkyl), -S-(alkenyl), -S-(alkynyl), -S-(aryl), -S-(heteroaryl), -S(O)-(alkyl), -S(O)-(aryl), -S(O)-(heteroaryl), -SO₂-(alkyl), -SO₂-(aryl), or -SO2-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
or a pharmaceutically acceptable salt thereof,
so as to thereby treat the neurological disorder.

The present disclosure yet further provides a method of treating a subject afflicted with a neurological disorder, wherein the neurological disorder is selected from the group consisting of Huntington’s disease, Rett syndrome, and CDKL5 disorder, and the other neurological disorders as described herein comprising administering to the subject an effective amount of a compound having the structure:

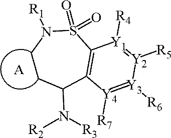

wherein
A is an aryl or heteroaryl, with or without substitution;
R₁ is —H or -(alkyl);
R₂ is -(alkyl), -(alkenyl), -(alkynyl), -(alkyl)-OH, -(alkyl)-CO₂H, -(alkyl)-CO₂-(alkyl), - (alkyl) —C(O) —NH₂, - (alkyl) -C(O) -NH(alkyl), -(alkyl)-C(O)-NH-(hydroxyalkyl), -(alkyl)-C(O)-N(alkyl)₂, -(alkyl)-C(O)-N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-O-(alkyl), -(alkyl)-(CH)-(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole); R₃ is —H or -(alkyl);
R₄, R₅, R₆ and R₇ are each absent or present, and when present, are each independently —H, —Cl, —Br, —F, —I, —CN, —CF₃, -OCF₃, -(alkyl), -(alkenyl), -(alkynyl), -(aryl), —NH₂, -NH-(alkyl), -NH-(alkenyl), -NH-(alkynyl), -NH-(aryl), -NH-(heteroaryl), -OH, —OAc, -O-C(O) (alkyl), -O-(alkyl), -O-(alkylaryl), -O-(alkenyl), -O-(alkynyl), -O-(aryl), -O-(heteroaryl), -S-(alkyl), -S-(alkenyl), -S-(alkynyl), -S-(aryl), -S-(heteroaryl), -S(O)-(alkyl), -S(O)-(aryl), -S(O)-(heteroaryl), -SO₂-(alkyl), -SO₂-(aryl), or -SO2-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
or a pharmaceutically acceptable salt thereof,
so as to thereby treat the neurological disorder.

The present disclosure yet further provides a method of treating a subject afflicted with a neurological disorder, wherein the neurological disorder is selected from the group consisting of Huntington’s disease, Rett syndrome, and CDKL5 disorder, and the other neurological disorders as described herein comprising administering to the subject an effective amount of a DOR agonist or a DOR antagonist and an effective amount of a compound having the structure:

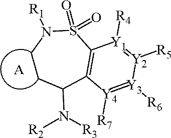

wherein
A is an aryl or heteroaryl, with or without substitution;
R₁ is —H or -(alkyl);
R₂ is -(alkyl), -(alkenyl), -(alkynyl), -(alkyl)-OH, -(alkyl)-CO₂H, -(alkyl)-CO₂-(alkyl), - (alkyl) —C(O) —NH₂, - (alkyl) -C(O)-NH(alkyl), -(alkyl)-C(O)-NH-(hydroxyalkyl), -(alkyl)-C(O)-N(alkyl)₂, -(alkyl)-C(O)-N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-O-(alkyl), -(alkyl)-(CH)-(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, -NH-(alkyl), -NH-(alkenyl), -NH-(alkynyl), -NH-(aryl), -NH-(heteroaryl), —OH, —OAc, —O—C(O) (alkyl), -O-(alkyl), -O-(alkylaryl), -O-(alkenyl), -O-(alkynyl), -O-(aryl), -O-(heteroaryl), -S-(alkyl), -S- (alkenyl), -S-(alkynyl), -S-(aryl), -S-(heteroaryl), -S(O)-(alkyl), -S(O)-(aryl), -S(O)- (heteroaryl), -SO₂-(alkyl), -SO₂-(aryl), or -SO2-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
or a pharmaceutically acceptable salt thereof,
so as to thereby treat the neurological disorder.

The present disclosure yet further provides a method of treating a subject afflicted with a neurological disorder, wherein the neurological disorder is selected from the group consisting of Huntington’s disease, Rett syndrome, and CDKL5 disorder, and the other neurological disorders as described herein comprising administering to the subject an effective amount of naloxone or methylnaltrexone and an effective amount of a compound having the structure:

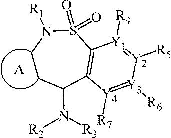

wherein
A is an aryl or heteroaryl, with or without substitution;
R₁ is —H or -(alkyl);
R₂ is -(alkyl), -(alkenyl), -(alkynyl), -(alkyl)-OH, -(alkyl)-CO₂H, -(alkyl)-CO₂-(alkyl), - (alkyl) —C(O) —NH₂, - (alkyl) -C(O) -NH(alkyl), -(alkyl)-C(O)-NH-(hydroxyalkyl), -(alkyl)-C(O)-N(alkyl)₂, -(alkyl)-C(O)-N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-O-(alkyl), -(alkyl)-(CH)-(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, -NH-(alkyl), -NH-(alkenyl), -NH-(alkynyl), -NH-(aryl), -NH-(heteroaryl), —OH, —OAc, -O-C(O)(alkyl), -O-(alkyl), -O-(alkylaryl), -O-(alkenyl), -O-(alkynyl), -O-(aryl), -O-(heteroaryl), -S-(alkyl), -S-(alkenyl), -S-(alkynyl), -S-(aryl), -S-(heteroaryl), -S(O)-(alkyl), -S(O)-(aryl), -S(O)-(heteroaryl), -SO₂-(alkyl), -SO₂-(aryl), or -SO2-(heteroaryl); and
Y₁, Y₂, Y₃ and Y₄ are each independently N or C,
   wherein when Yᵢ is N, then R₄ is absent, and when Yᵢ is C, then R₄ is present; when Y₂ is N, then Rₛ is absent, and when Y₂ is C, then Rₛ is present; when Y₃ is N, then R₆ is absent, and when Y₃ is C, then R₆ is present; when Y₄ is N, then R₇ is absent, and when Y₄ is C, then R₇ is present,
or a pharmaceutically acceptable salt thereof,
so as to thereby treat the neurological disorder.

The present disclosure yet further provides a method of treating a subject afflicted with a neurological disorder, wherein the neurological disorder is Huntington’s disease, Rett syndrome, or CDKL5 disorder comprising administering to the subject an effective amount of naloxone or methylnaltrexone and an effective amount of a compound having the structure:

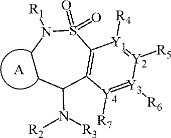

wherein
A is an aryl or heteroaryl, with or without substitution;
R₁ is —H or -(alkyl);
R₂ is -(alkyl), -(alkenyl), -(alkynyl), -(alkyl)-OH, -(alkyl)-CO₂H, -(alkyl)-CO₂-(alkyl), - (alkyl) —C (O) —NH₂, - (alkyl) -C (O) -NH(alkyl), -(alkyl)-C(O)-NH-(hydroxyalkyl), -(alkyl)-C(O)-N(alkyl)₂, -(alkyl)-C(O)-N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-O-(alkyl), -(alkyl)-(CH)-(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, —NH—(alkyl), -NH-(alkenyl), -NH-(alkynyl), -NH-(aryl), -NH-(heteroaryl), -OH, —OAc, -O-C(O)(alkyl), -O-(alkyl), -O-(alkylaryl), -O-(alkenyl), -O-(alkynyl), -O-(aryl), -O-(heteroaryl), -S-(alkyl), -S-(alkenyl), -S-(alkynyl), -S-(aryl), -S-(heteroaryl), -S(O)-(alkyl), -S(O)-(aryl), -S(O)-(heteroaryl), -SO₂-(alkyl), -SO₂-(aryl), or -SO2-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
or a pharmaceutically acceptable salt thereof, so as to thereby treat the neurological disorder.

The above group of compounds, including specific compounds within the genus and synthetic methods for their preparation, are disclosed in PCT International Application No. PCT/US2015/020273, filed Mar. 12, 2015, the contents of which are hereby incorporated by reference.

In one embodiment of any of the compounds disclosed herein R₂ is -(C₂₋₅ alkyl) —CO₂H or any combination of any of - (C₂ alkyl) —CO₂H, - (C₃ alkyl) —CO₂H, -(C₄ alkyl)-CO₂H, or - (C₅ alkyl)-CO₂H.

In one embodiment of any of the compounds disclosed herein R₂ is - (C₂₋₅ alkyl) -CO₂- (alkyl) or any combination of any of - (C₂ alkyl) -CO₂-(alkyl), -(C₃ alkyl) -CO₂- (alkyl), -(C₄ alkyl) -CO₂- (alkyl) or - (C₅ alkyl)-CO₂-(alkyl).

The present disclosure provides a pharmaceutical composition comprising a compound of the present disclosure and a pharmaceutically acceptable carrier for treating or preventing a neurological disorder, wherein the neurological disorder is selected from the group consisting of Huntington’s disease, Rett syndrome, and CDKL5 disorder, and the other neurological disorders as described herein.

The present disclosure provides a pharmaceutical composition comprising a compound of this disclosure and an NMDA receptor antagonist, an NMDA receptor partial agonist, a neurokinin 1 receptor antagonist, a neurokinin 2 receptor antagonist, a neurokinin 3 receptor antagonist, a DOR agonist, a DOR antagonist, naloxone or methylnaltrexone and a pharmaceutically acceptable carrier for treating or preventing a neurological disorder, wherein the neurological disorder is selected from the group consisting of Huntington’s disease, Rett syndrome, and CDKL5 disorder, and the other neurological disorders as described herein.

The present disclosure provides a method of activating the mu-opioid receptor comprising contacting the mu-opioid receptor with a compound of this disclosure, thereby treating or preventing a neurological disorder, wherein the neurological disorder is selected from the group consisting of Huntington’s disease, Rett syndrome, and CDKL5 disorder, and the other neurological disorders as described herein.

The present disclosure provides a method of activating the delta-opioid receptor comprising contacting the delta-opioid receptor with a compound of this disclosure, thereby treating or preventing a neurological disorder, wherein the neurological disorder is selected from the group consisting of Huntington’s disease, Rett syndrome, and CDKL5 disorder, and the other neurological disorders as described herein.

The present disclosure provides a method of treating a subject afflicted with a neurological disorder, wherein the neurological disorder is Huntington’s disease n comprising administering an effective amount of a compound of this disclosure to the subject so as to treat the neurological disorder.

The present disclosure provides a method of treating a subject afflicted with a neurological disorder, wherein the neurological disorder is Rett syndrome comprising administering an effective amount of a compound of this disclosure to the subject so as to treat the neurological disorder.

The present disclosure provides a method of treating a subject afflicted with a neurological disorder, wherein the neurological disorder is CDKL5 disorder comprising administering an effective amount of a compound of this disclosure to the subject so as to treat the neurological disorder.

In some embodiments, the mu-opioid receptors or delta-opioid receptors are in a human subject.

The present disclosure also provides a compound having the structure:

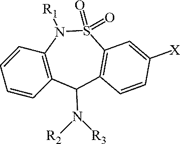

wherein
R₁ is —H or -(alkyl);
R₂ is -(alkyl)-CO₂H or -(alkyl)-CO₂-(alkyl);
R₃ is —H or -(alkyl); and
X is —Br or —I,
or a salt or ester thereof, for use as an add-on therapy or in combination with an NMDA receptor antagonist, an NMDA receptor partial agonist, a neurokinin 1 receptor antagonist, a neurokinin 2 receptor antagonist, a neurokinin 3 receptor antagonist, or a DOR agonist in treating a subject afflicted with a neurological disorder, wherein the neurological disorder is selected from the group consisting of Huntington’s disease, Rett syndrome, and CDKL5 disorder, and the other neurological disorders as described herein.

The present disclosure also provides a compound having the structure:

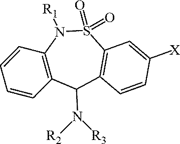

wherein
R₁ is —H or -(alkyl);
R₂ is -(alkyl)-CO₂H or -(alkyl)-CO₂-(alkyl);
R₃ is —H or -(alkyl); and
X is —Br or —I,
or a salt or ester thereof, for use as an add-on therapy or in combination with an NMDA receptor antagonist, an NMDA receptor partial agonist, a neurokinin 1 receptor antagonist or a DOR agonist in treating a subject afflicted with a neurological disorder, wherein the neurological disorder is selected from the group consisting of Huntington’s disease, Rett syndrome, and CDKL5 disorder, and the other neurological disorders as described herein.

The present disclosure also provides a compound having the structure:

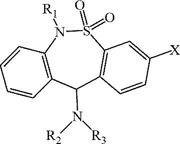

wherein
R₁ is —H or -(alkyl);
R₂ is -(alkyl)-CO₂H or -(alkyl)-CO₂-(alkyl);
R₃ is —H or -(alkyl); and
X is —Br or —I,
or a salt or ester thereof, for use as an add-on therapy or in combination with an NMDA receptor antagonist, an NMDA receptor partial agonist, a neurokinin 1 receptor antagonist, a neurokinin 2 receptor antagonist, a neurokinin 3 receptor antagonist, or a DOR agonist in treating a subject afflicted with a neurological disorder, wherein the neurological disorder is selected from the group consisting of Huntington’s disease, Rett syndrome, and CDKL5 disorder, and the other neurological disorders as described herein.

The present disclosure also provides a compound having the structure:

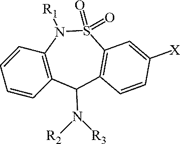

wherein
R₁ is —H or -(alkyl);
R₂ is -(alkyl)-CO₂H or -(alkyl)-CO₂-(alkyl);
R₃ is —H or -(alkyl); and
X is —Br or —I,
or a salt or ester thereof, for use as an add-on therapy or in combination with a DOR antagonist in treating a subject afflicted with a neurological disorder, wherein the neurological disorder is selected from the group consisting of Huntington’s disease, Rett syndrome, and CDKL5 disorder, and the other neurological disorders as described herein.

The present disclosure also provides a compound having the structure:

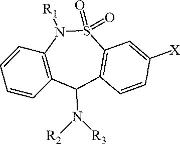

wherein
R₁ is —H or -(alkyl);
R₂ is -(alkyl)-CO₂H or -(alkyl)-CO₂-(alkyl);
R₃ is —H or -(alkyl); and
X is —Br or —I,
or a salt or ester thereof, for use as an add-on therapy or in combination with a DOR antagonist in treating a subject afflicted with a neurological disorder, wherein the neurological disorder is selected from the group consisting of Huntington’s disease, Rett syndrome, and CDKL5 disorder, and the other neurological disorders as described herein.

The present disclosure also provides a compound having the structure:

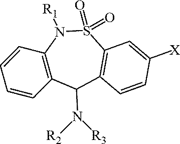

wherein
R₁ is —H or -(alkyl);
R₂ is -(alkyl)-CO₂H or -(alkyl)-CO₂-(alkyl);
R₃ is —H or -(alkyl); and
X is —Br or —I,
or a salt or ester thereof, for use as an add-on therapy or in combination with an NMDA receptor antagonist, an NMDA receptor partial agonist, a neurokinin 1 receptor antagonist, a DOR agonist or a DOR antagonist in treating a subject afflicted with a neurological disorder, wherein the neurological disorder is selected from the group consisting of Huntington’s disease, Rett syndrome, and CDKL5 disorder, and the other neurological disorders as described herein.

The present disclosure also provides a compound having the structure:

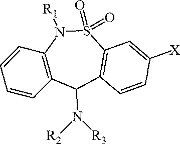

wherein
R₁ is —H or -(alkyl);
R₂ is -(alkyl)-CO₂H or -(alkyl)-CO₂-(alkyl);
R₃ is —H or -(alkyl); and
X is —Br or —I,
or a salt or ester thereof, for use as an add-on therapy or in combination with a DOR agonist or a DOR antagonist in treating a subject afflicted with a neurological disorder, wherein the neurological disorder is selected from the group consisting of Huntington’s disease, Rett syndrome, and CDKL5 disorder, and the other neurological disorders as described herein.

The present disclosure also provides a compound having the structure:

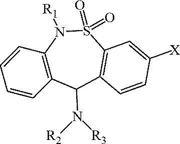

wherein
R₁ is —H or -(alkyl);
R₂ is -(alkyl)-CO₂H or - (alkyl) —CO₂— (alkyl) ;
R₃ is —H or -(alkyl); and
X is —Br or —I,
or a salt or ester thereof, for use as an add-on therapy or in combination with an NMDA receptor antagonist, an NMDA receptor partial agonist, a neurokinin 1 receptor antagonist or a DOR antagonist in treating a subject afflicted with a neurological disorder, wherein the neurological disorder is selected from the group consisting of Huntington’s disease, Rett syndrome, and CDKL5 disorder, and the other neurological disorders as described herein.

The present disclosure also provides a compound having the structure:

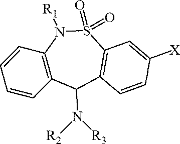

wherein
R₁ is —H or -(alkyl);
R₂ is -(alkyl)-CO₂H or -(alkyl)-CO₂-(alkyl);
R₃ is —H or -(alkyl); and
X is —Br or —I,
or a salt or ester thereof, for use as an add-on therapy or in combination with a DOR agonist or a DOR antagonist in treating a subject afflicted with a neurological disorder, wherein the neurological disorder is selected from the group consisting of Huntington’s disease, Rett syndrome, and CDKL5 disorder, and the other neurological disorders as described herein.

The present disclosure further provides a pharmaceutical composition comprising an amount of a compound having the structure:

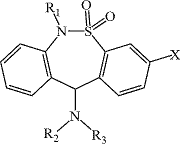

wherein
R₁ is —H or -(alkyl);
R₂ is -(alkyl)-CO₂H or -(alkyl)-CO₂-(alkyl);
R₃ is —H or -(alkyl); and
X is —Br or —I,
or a salt or ester thereof, and an amount of a NMDA receptor antagonist, an NMDA receptor partial agonist, a neurokinin 1 receptor antagonist, a neurokinin 2 receptor antagonist, a neurokinin 3 receptor antagonist, or a DOR agonist for use in treating a subject afflicted with a neurological disorder, wherein the neurological disorder is selected from the group consisting of Huntington’s disease, Rett syndrome, and CDKL5 disorder, and the other neurological disorders as described herein.

The present disclosure further provides a pharmaceutical composition comprising an amount of a compound having the structure

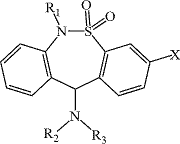

wherein
R₁ is —H or -(alkyl);
R₂ is -(alkyl)-CO₂H or -(alkyl)-CO₂-(alkyl);
R₃ is —H or -(alkyl); and
X is —Br or —I,
or a salt or ester thereof, and an amount of a NMDA receptor antagonist, an NMDA receptor partial agonist, a neurokinin 1 receptor antagonist or a DOR agonist for use in treating a subject afflicted with a neurological disorder, wherein the neurological disorder is selected from the group consisting of Huntington’s disease, Rett syndrome, and CDKL5 disorder, and the other neurological disorders as described herein.

The present disclosure further provides a pharmaceutical composition comprising an amount of a compound having the structure

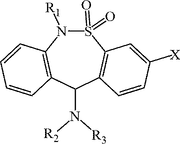

wherein
R₁ is —H or -(alkyl);
R₂ is -(alkyl)-CO₂H or -(alkyl)-CO₂-(alkyl);
R₃ is —H or -(alkyl); and
X is —Br or —I,
or a salt or ester thereof, and an amount of a DOR antagonist for use in treating a subject afflicted with a neurological disorder, wherein the neurological disorder is selected from the group consisting of Huntington’s disease, Rett syndrome, and CDKL5 disorder, and the other neurological disorders as described herein.

The present disclosure further provides a pharmaceutical composition comprising an amount of a compound having the structure

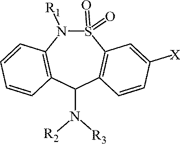

wherein
R₁ is —H or -(alkyl);
R₂ is - (alkyl) —CO₂H or - (alkyl) —CO₂— (alkyl) ;
R₃ is —H or -(alkyl); and
X is —Br or —I,
or a salt or ester thereof, and an amount of a NMDA receptor antagonist, an NMDA receptor partial agonist, a neurokinin 1 receptor antagonist, a neurokinin 2 receptor antagonist, a neurokinin 3 receptor antagonist, or a DOR agonist for use in treating a subject afflicted with a neurological disorder, wherein the neurological disorder is selected from the group consisting of Huntington’s disease, Rett syndrome, and CDKL5 disorder, and the other neurological disorders as described herein.

The present disclosure further provides a pharmaceutical composition comprising an amount of a compound having the structure

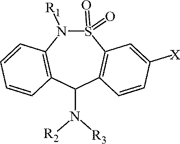

wherein
R₁ is —H or -(alkyl);
R₂ is -(alkyl)-CO₂H or -(alkyl)-CO₂-(alkyl);
R₃ is —H or -(alkyl); and
X is —Br or —I,
or a salt or ester thereof, and an amount of a DOR antagonist for use in treating a subject afflicted with a neurological disorder, wherein the neurological disorder is selected from the group consisting of Huntington’s disease, Rett syndrome, and CDKL5 disorder, and the other neurological disorders as described herein.

The present disclosure further provides a pharmaceutical composition comprising an amount of a compound having the structure

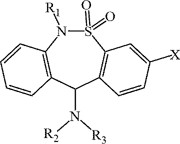

wherein
R₁ is —H or -(alkyl);
R₂ is -(alkyl)-CO₂H or -(alkyl)-CO₂-(alkyl);
R₃ is —H or -(alkyl); and
X is —Br or —I,
or a salt or ester thereof, and an amount of a NMDA receptor antagonist, an NMDA receptor partial agonist, a neurokinin 1 receptor antagonist, a DOR agonist or a DOR antagonist in treating a subject afflicted with a neurological disorder, wherein the neurological disorder is selected from the group consisting of Huntington’s disease, Rett syndrome, and CDKL5 disorder, and the other neurological disorders as described herein.

The present disclosure further provides a pharmaceutical composition comprising an amount of a compound having the structure

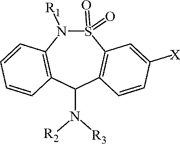

wherein
R₁ is —H or -(alkyl);
R₂ is -(alkyl)-CO₂H or -(alkyl)-CO₂-(alkyl);
R₃ is —H or -(alkyl); and
X is —Br or —I, or a salt or ester thereof, and an amount of a DOR agonist or a DOR antagonist in treating a subject afflicted with a neurological disorder, wherein the neurological disorder is selected from the group consisting of Huntington’s disease, Rett syndrome, and CDKL5 disorder, and the other neurological disorders as described herein.

The present disclosure further provides a pharmaceutical composition comprising an amount of a compound having the structure

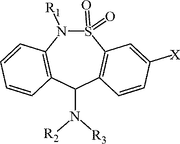

wherein
R₁ is —H or -(alkyl);
R₂ is -(alkyl)-CO₂H or -(alkyl)-CO₂-(alkyl);
R₃ is —H or -(alkyl); and
X is —Br or —I,
or a salt or ester thereof, and an amount of an NMDA receptor antagonist, an NMDA receptor partial agonist, a neurokinin 1 receptor antagonist or a DOR antagonist in treating a subject afflicted with a neurological disorder, wherein the neurological disorder is selected from the group consisting of Huntington’s disease, Rett syndrome, and CDKL5 disorder, and the other neurological disorders as described herein.

The present disclosure further provides a pharmaceutical composition comprising an amount of a compound having the structure

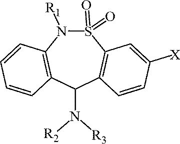

wherein
R₁ is —H or -(alkyl);
R₂ is -(alkyl)-CO₂H or -(alkyl)-CO₂-(alkyl);
R₃ is —H or -(alkyl); and
X is —Br or —I,
or a salt or ester thereof, and an amount of a DOR agonist or a DOR antagonist in treating a subject afflicted with a neurological disorder, wherein the neurological disorder is selected from the group consisting of Huntington’s disease, Rett syndrome, and CDKL5 disorder, and the other neurological disorders as described herein.

The present disclosure also provides a compound having the structure:

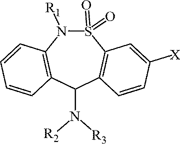

wherein
R₁ is —H or -(alkyl);
R₂ is -(alkyl)-CO₂H or -(alkyl)-CO₂-(alkyl);
R₃ is —H or -(alkyl); and
X is —Br or —I,
or a salt or ester thereof, for use as an add-on therapy or in combination with an SSRI or an SNRI in treating a subject afflicted with a neurological disorder, wherein the neurological disorder is selected from the group consisting of Huntington’s disease, Rett syndrome, and CDKL5 disorder, and the other neurological disorders as described herein.

The present disclosure further provides a pharmaceutical composition comprising an amount of a compound having the structure

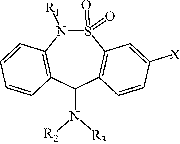

wherein
R₁ is —H or -(alkyl);
R₂ is -(alkyl)-CO₂H or -(alkyl)-CO₂-(alkyl);
R₃ is —H or -(alkyl); and
X is —Br or —I,
or a salt or ester thereof, and an amount of an SSRI or an SNRI in treating a subject afflicted with a neurological disorder, wherein the neurological disorder is selected from the group consisting of Huntington’s disease, Rett syndrome, and CDKL5 disorder, and the other neurological disorders as described herein.

In some embodiments, a package comprising:
a) a first pharmaceutical composition comprising an amount of an NMDA receptor antagonist, an NMDA receptor partial agonist, a neurokinin 1 receptor antagonist or a DOR agonist and a pharmaceutically acceptable carrier;
b) a second pharmaceutical composition comprising an amount of any of the compounds of the present disclosure, or a salt or ester thereof; and
c) instructions for use of the first and second pharmaceutical compositions together to treat a subject afflicted with a neurological disorder, wherein the neurological disorder is selected from the group consisting of Huntington’s disease, Rett syndrome, and CDKL5 disorder, and the other neurological disorders as described herein.

In some embodiments, a package comprising:
a) a first pharmaceutical composition comprising an amount of a DOR antagonist and a pharmaceutically acceptable carrier;
b) a second pharmaceutical composition comprising an amount of any of the compounds of the present disclosure, or a salt or ester thereof; and
c) instructions for use of the first and second pharmaceutical compositions together to treat a subject afflicted with a neurological disorder, wherein the neurological disorder is selected from the group consisting of Huntington’s disease, Rett syndrome, and CDKL5 disorder, and the other neurological disorders as described herein.

In some embodiments, a package comprising:
a) a first pharmaceutical composition comprising an amount of an NMDA receptor antagonist, an NMDA receptor partial agonist, a neurokinin 1 receptor antagonist, a DOR agonist or a DOR antagonist and a pharmaceutically acceptable carrier;
b) a second pharmaceutical composition comprising an amount of any of the compounds of the present disclosure, or a salt or ester thereof; and
c) instructions for use of the first and second pharmaceutical compositions together to treat a subject afflicted with a neurological disorder, wherein the neurological disorder is selected from the group consisting of Huntington’s disease, Rett syndrome, and CDKL5 disorder, and the other neurological disorders as described herein.

In some embodiments, a package comprising:
a) a first pharmaceutical composition comprising an amount of a DOR agonist or a DOR antagonist and a pharmaceutically acceptable carrier;
b) a second pharmaceutical composition comprising an amount of any of the compounds of the present disclosure, or a salt or ester thereof; and
c) instructions for use of the first and second pharmaceutical compositions together to treat a subject afflicted with a neurological disorder, wherein the neurological disorder is selected from the group consisting of Huntington’s disease, Rett syndrome, and CDKL5 disorder, and the other neurological disorders as described herein.

In some embodiments, a package comprising:
a) a first pharmaceutical composition comprising an amount of an NMDA receptor antagonist, an NMDA receptor partial agonist, a neurokinin 1 receptor antagonist, or a DOR antagonist and a pharmaceutically acceptable carrier;
b) a second pharmaceutical composition comprising an amount of any of the compounds of the present disclosure, or a salt or ester thereof; and
c) instructions for use of the first and second pharmaceutical compositions together to treat a subject afflicted with a neurological disorder, wherein the neurological disorder is selected from the group consisting of Huntington’s disease, Rett syndrome, and CDKL5 disorder, and the other neurological disorders as described herein.

In some embodiments, a package comprising:
a) a first pharmaceutical composition comprising an amount of a DOR agonist or a DOR antagonist and a pharmaceutically acceptable carrier;
b) a second pharmaceutical composition comprising an amount of any of the compounds of the present disclosure, or a salt or ester thereof; and
c) instructions for use of the first and second pharmaceutical compositions together to treat a subject afflicted with a neurological disorder, wherein the neurological disorder is selected from the group consisting of Huntington’s disease, Rett syndrome, and CDKL5 disorder, and the other neurological disorders as described herein.

In some embodiments, a therapeutic package for dispensing to, or for use in dispensing to, a subject afflicted with a neurological disorder, wherein the neurological disorder is selected from the group consisting of Huntington’s disease, Rett syndrome, and CDKL5 disorder, and the other neurological disorders as described herein, which comprises:
(a) one or more unit doses, each such unit dose comprising:
   (i)an amount of any of the compounds of the present disclosure, or a salt or ester thereof; and
   (ii) an amount of an NMDA receptor antagonist, an NMDA receptor partial agonist, a neurokinin 1 receptor antagonist or a DOR agonist,
      wherein the respective amounts of said compound(s) and said agonist or antagonist in said unit dose are effective, upon concomitant administration to said subject, to treat the disorder, and
(b) a finished pharmaceutical container therefor, said container containing said unit dose or unit doses, said container further containing or comprising labeling directing the use of said package in the treatment of said disorder.

In some embodiments, a therapeutic package for dispensing to, or for use in dispensing to, a subject afflicted with a neurological disorder, wherein the neurological disorder is selected from the group consisting of Huntington’s disease, Rett syndrome, and CDKL5 disorder, and the other neurological disorders as described herein, which comprises:
(a) one or more unit doses, each such unit dose comprising:
   (i)an amount of any of the compounds of the present disclosure, or a salt or ester thereof; and
   (ii) an amount of a DOR antagonist,
      wherein the respective amounts of said compound(s) and said antagonist in said unit dose are effective, upon concomitant administration to said subject, to treat the disorder, and
(b) a finished pharmaceutical container therefor, said container containing said unit dose or unit doses, said container further containing or comprising labeling directing the use of said package in the treatment of said disorder.

In some embodiments, a therapeutic package for dispensing to, or for use in dispensing to, a subject afflicted with a neurological disorder, wherein the neurological disorder is selected from the group consisting of Huntington’s disease, Rett syndrome, and CDKL5 disorder, and the other neurological disorders as described herein, which comprises:
(a) one or more unit doses, each such unit dose comprising:
   (i)an amount of any of the compounds of the present disclosure, or a salt or ester thereof; and
   (ii) an amount of an NMDA receptor antagonist, an NMDA receptor partial agonist, a neurokinin 1 receptor antagonist, a DOR agonist or a DOR antagonist,
      wherein the respective amounts of said compound(s) and said agonist or antagonist in said unit dose are effective, upon concomitant administration to said subject, to treat the disorder, and
(b) a finished pharmaceutical container therefor, said container containing said unit dose or unit doses, said container further containing or comprising labeling directing the use of said package in the treatment of said disorder.

In some embodiments, a therapeutic package for dispensing to, or for use in dispensing to, a subject afflicted with a neurological disorder, wherein the neurological disorder is selected from the group consisting of Huntington’s disease, Rett syndrome, and CDKL5 disorder, and the other neurological disorders as described herein, which comprises:
(a) one or more unit doses, each such unit dose comprising:
   (i)an amount of any of the compounds of the present disclosure, or a salt or ester thereof; and
   (ii) an amount of a DOR agonist or a DOR antagonist,
      wherein the respective amounts of said compound(s) and said agonist or antagonist in said unit dose are effective, upon concomitant administration to said subject, to treat the disorder, and
(b) a finished pharmaceutical container therefor, said container containing said unit dose or unit doses, said container further containing or comprising labeling directing the use of said package in the treatment of said disorder.

In some embodiments, a therapeutic package for dispensing to, or for use in dispensing to, a subject afflicted with a neurological disorder, wherein the neurological disorder is selected from the group consisting of Huntington’s disease, Rett syndrome, and CDKL5 disorder, and the other neurological disorders as described herein, which comprises:
(a) one or more unit doses, each such unit dose comprising:
   (i)an amount of any of the compounds of the present disclosure, or a salt or ester thereof; and
   (ii) an amount of an NMDA receptor antagonist, an NMDA receptor partial agonist, a neurokinin 1 receptor antagonist, or a DOR antagonist,
      wherein the respective amounts of said compound(s) and said agonist or antagonist in said unit dose are effective, upon concomitant administration to said subject, to treat the disorder, and
(b) a finished pharmaceutical container therefor, said container containing said unit dose or unit doses, said container further containing or comprising labeling directing the use of said package in the treatment of said disorder.

In some embodiments, a therapeutic package for dispensing to, or for use in dispensing to, a subject afflicted with a neurological disorder, wherein the neurological disorder is selected from the group consisting of Huntington’s disease, Rett syndrome, and CDKL5 disorder, and the other neurological disorders as described herein, which comprises:
(a) one or more unit doses, each such unit dose comprising:
   (i)an amount of any of the compounds of the present disclosure, or a salt or ester thereof; and
   (ii) an amount of a DOR agonist or a DOR antagonist,
      wherein the respective amounts of said compound(s) and said agonist or antagonist in said unit dose are effective, upon concomitant administration to said subject, to treat the disorder, and
(b) a finished pharmaceutical container therefor, said container containing said unit dose or unit doses, said container further containing or comprising labeling directing the use of said package in the treatment of said disorder.

The therapeutic package of the above embodiment, wherein the respective amounts of said compound(s) and said agonist or antagonist in said unit dose when taken together is more effective to treat the disorder than when compared to the administration of said compound(s) in the absence of said agonist or antagonist or the administration of said agonist or antagonist in the absence of said compound(s).

A pharmaceutical composition in unit dosage form, useful in treating a subject afflicted with a neurological disorder, wherein the neurological disorder is selected from the group consisting of Huntington’s disease, Rett syndrome, and CDKL5 disorder, and the other neurological disorders as described herein, which comprises:
(i) an amount of any of the compounds of the present disclosure, or a salt or ester thereof; and
(ii) an amount of an NMDA receptor antagonist, an NMDA receptor partial agonist, a neurokinin 1 receptor antagonist, a neurokinin 2 receptor antagonist, a neurokinin 3 receptor antagonist, or a DOR agonist,
   wherein the respective amounts of said compound(s) and said agonist or antagonist in said composition are effective, upon concomitant administration to said subject of one or more of said unit dosage forms of said composition, to treat the disorder.

A pharmaceutical composition in unit dosage form, useful in treating a subject afflicted with a neurological disorder, wherein the neurological disorder is selected from the group consisting of Huntington’s disease, Rett syndrome, and CDKL5 disorder, and the other neurological disorders as described herein, which comprises:
(i) an amount of any of the compounds of the present disclosure, or a salt or ester thereof; and
(ii) an amount of a DOR antagonist,
   wherein the respective amounts of said compound(s) and said antagonist in said composition are effective, upon concomitant administration to said subject of one or more of said unit dosage forms of said composition, to treat the disorder.

The pharmaceutical composition of the above embodiment, wherein the respective amounts of said compound(s) and said agonist or antagonist in said unit dose when taken together is more effective to treat the disorder than when compared to the administration of said compound(s) in the absence of said agonist or antagonist or the administration of said agonist or antagonist in the absence of said compound(s).

In some embodiments, a package comprising:
a) a first pharmaceutical composition comprising an amount of an NMDA receptor antagonist, an NMDA receptor partial agonist, a neurokinin 1 receptor antagonist, a neurokinin 2 receptor antagonist, a neurokinin 3 receptor antagonist, or a DOR agonist and a pharmaceutically acceptable carrier;
b) a second pharmaceutical composition comprising an amount of any of the compounds of the present disclosure, or a salt or ester thereof; and
c) instructions for use of the first and second pharmaceutical compositions together to treat a subject afflicted with a neurological disorder, wherein the neurological disorder is selected from the group consisting of Huntington’s disease, Rett syndrome, and CDKL5 disorder, and the other neurological disorders as described herein.

In some embodiments, a package comprising:
a) a first pharmaceutical composition comprising a DOR antagonist and a pharmaceutically acceptable carrier;
b) a second pharmaceutical composition comprising an amount of any of the compounds of the present disclosure, or a salt or ester thereof; and
c) instructions for use of the first and second pharmaceutical compositions together to treat a subject afflicted with a neurological disorder, wherein the neurological disorder is selected from the group consisting of Huntington’s disease, Rett syndrome, and CDKL5 disorder, and the other neurological disorders as described herein.

In some embodiments, a package comprising:
a) a first pharmaceutical composition comprising an amount of an NMDA receptor antagonist, an NMDA receptor partial agonist, a neurokinin 1 receptor antagonist, a DOR agonist or a DOR antagonist and a pharmaceutically acceptable carrier;
b) a second pharmaceutical composition comprising an amount of any of the compounds of the present disclosure, or a salt or ester thereof; and
c) instructions for use of the first and second pharmaceutical compositions together to treat a subject afflicted with a neurological disorder, wherein the neurological disorder is selected from the group consisting of Huntington’s disease, Rett syndrome, and CDKL5 disorder, and the other neurological disorders as described herein.

In some embodiments, a package comprising:
a) a first pharmaceutical composition comprising an amount of a DOR agonist or a DOR antagonist and a pharmaceutically acceptable carrier;
b) a second pharmaceutical composition comprising an amount of any of the compounds of the present disclosure, or a salt or ester thereof; and
c) instructions for use of the first and second pharmaceutical compositions together to treat a subject afflicted with a neurological disorder, wherein the neurological disorder is selected from the group consisting of Huntington’s disease, Rett syndrome, and CDKL5 disorder, and the other neurological disorders as described herein.

In some embodiments, a package comprising:
a) a first pharmaceutical composition comprising an amount of an NMDA receptor antagonist, an NMDA receptor partial agonist, a neurokinin 1 receptor antagonist or a DOR antagonist and a pharmaceutically acceptable carrier;
b) a second pharmaceutical composition comprising an amount of any of the compounds of the present disclosure, or a salt or ester thereof; and
c) instructions for use of the first and second pharmaceutical compositions together to treat a subject afflicted with a neurological disorder, wherein the neurological disorder is selected from the group consisting of Huntington’s disease, Rett syndrome, and CDKL5 disorder, and the other neurological disorders as described herein.

In some embodiments, a package comprising:
a) a first pharmaceutical composition comprising an amount of a DOR agonist or a DOR antagonist and a pharmaceutically acceptable carrier;
b) a second pharmaceutical composition comprising an amount of any of the compounds of the present disclosure, or a salt or ester thereof; and
c) instructions for use of the first and second pharmaceutical compositions together to treat a subject afflicted with a neurological disorder, wherein the neurological disorder is Huntington’s disease.

In some embodiments, a package comprising:
a) a first pharmaceutical composition comprising an amount of a DOR agonist or a DOR antagonist and a pharmaceutically acceptable carrier;
b) a second pharmaceutical composition comprising an amount of any of the compounds of the present disclosure, or a salt or ester thereof; and
c) instructions for use of the first and second pharmaceutical compositions together to treat a subject afflicted with a neurological disorder, wherein the neurological disorder is Rett syndrome.

In some embodiments, a package comprising:
a) a first pharmaceutical composition comprising an amount of a DOR agonist or a DOR antagonist and a pharmaceutically acceptable carrier;
b) a second pharmaceutical composition comprising an amount of any of the compounds of the present disclosure, or a salt or ester thereof; and
c) instructions for use of the first and second pharmaceutical compositions together to treat a subject afflicted with a neurological disorder, wherein the neurological disorder is CDKL5 disorder.

In some embodiments, a therapeutic package for dispensing to, or for use in dispensing to, a subject afflicted with a neurological disorder, wherein the neurological disorder is selected from the group consisting of Huntington’s disease, Rett syndrome, and CDKL5 disorder, and the other neurological disorders as described herein, which comprises:
(a) one or more unit doses, each such unit dose comprising:
   (i) an amount of any of the compounds of the present disclosure, or a salt or ester thereof; and
   (ii) an amount of an NMDA receptor antagonist, an NMDA receptor partial agonist, a neurokinin 1 receptor antagonist, a neurokinin 2 receptor antagonist, a neurokinin 3 receptor antagonist, or a DOR agonist,
      wherein the respective amounts of said compound(s) and said agonist or antagonist in said unit dose are effective, upon concomitant administration to said subject, to treat the disorder, and
(b) a finished pharmaceutical container therefor, said container containing said unit dose or unit doses, said container further containing or comprising labeling directing the use of said package in the treatment of said disorder.

In some embodiments, a therapeutic package for dispensing to, or for use in dispensing to, a subject afflicted with a neurological disorder, wherein the neurological disorder is selected from the group consisting of Huntington’s disease, Rett syndrome, and CDKL5 disorder, and the other neurological disorders as described herein, which comprises:
(a) one or more unit doses, each such unit dose comprising:
   (i) an amount of any of the compounds of the present disclosure, or a salt or ester thereof; and
   (ii) an amount of a DOR antagonist,
      wherein the respective amounts of said compound(s) and said antagonist in said unit dose are effective, upon concomitant administration to said subject, to treat the disorder, and
(b) a finished pharmaceutical container therefor, said container containing said unit dose or unit doses, said container further containing or comprising labeling directing the use of said package in the treatment of said disorder.

In some embodiments, a therapeutic package for dispensing to, or for use in dispensing to, a subject afflicted with a neurological disorder, wherein the neurological disorder is selected from the group consisting of Huntington’s disease, Rett syndrome, and CDKL5 disorder, and the other neurological disorders as described herein, which comprises:
(a) one or more unit doses, each such unit dose comprising:
   (i) an amount of any of the compounds of the present disclosure, or a salt or ester thereof; and
   (ii) an amount of an NMDA receptor antagonist, an NMDA receptor partial agonist, a neurokinin 1 receptor antagonist, a DOR agonist or a DOR antagonist,
      wherein the respective amounts of said compound(s) and said agonist or antagonist in said unit dose are effective, upon concomitant administration to said subject, to treat the disorder, and
(b) a finished pharmaceutical container therefor, said container containing said unit dose or unit doses, said container further containing or comprising labeling directing the use of said package in the treatment of said disorder.

In some embodiments, a therapeutic package for dispensing to, or for use in dispensing to, a subject afflicted with a neurological disorder, wherein the neurological disorder is selected from the group consisting of Huntington’s disease, Rett syndrome, and CDKL5 disorder, and the other neurological disorders as described herein, which comprises:
a) one or more unit doses, each such unit dose comprising:
   (i) an amount of any of the compounds of the present disclosure, or a salt or ester thereof; and
   (ii) an amount of a DOR agonist or a DOR antagonist,
      wherein the respective amounts of said compound(s) and said agonist or antagonist in said unit dose are effective, upon concomitant administration to said subject, to treat the disorder, and
(b) a finished pharmaceutical container therefor, said container containing said unit dose or unit doses, said container further containing or comprising labeling directing the use of said package in the treatment of said disorder.

In some embodiments, a therapeutic package for dispensing to, or for use in dispensing to, a subject afflicted with a neurological disorder, wherein the neurological disorder is selected from the group consisting of Huntington’s disease, Rett syndrome, and CDKL5 disorder, and the other neurological disorders as described herein, which comprises:
a) one or more unit doses, each such unit dose comprising:
   (i) an amount of any of the compounds of the present disclosure, or a salt or ester thereof; and
   (ii) an amount of an NMDA receptor antagonist, an NMDA receptor partial agonist, a neurokinin 1 receptor antagonist, or a DOR antagonist,
      wherein the respective amounts of said compound(s) and said agonist or antagonist in said unit dose are effective, upon concomitant administration to said subject, to treat the disorder, and
(b) a finished pharmaceutical container therefor, said container containing said unit dose or unit doses, said container further containing or comprising labeling directing the use of said package in the treatment of said disorder.

In some embodiments, a therapeutic package for dispensing to, or for use in dispensing to, a subject afflicted with a neurological disorder, wherein the neurological disorder is selected from the group consisting of Huntington’s disease, Rett syndrome, and CDKL5 disorder, and the other neurological disorders as described herein, which comprises:
a) one or more unit doses, each such unit dose comprising:
   (i) an amount of any of the compounds of the present disclosure, or a salt or ester thereof; and
   (ii) an amount of a DOR agonist or a DOR antagonist,
      wherein the respective amounts of said compound(s) and said agonist or antagonist in said unit dose are effective, upon concomitant administration to said subject, to treat the disorder, and
(b) a finished pharmaceutical container therefor, said container containing said unit dose or unit doses, said container further containing or comprising labeling directing the use of said package in the treatment of said disorder.

The therapeutic package of the above embodiment, wherein the respective amounts of said compound(s) and said agonist or antagonist in said unit dose when taken together is more effective to treat the disorder than when compared to the administration of said compound(s) in the absence of said agonist or antagonist or the administration of said agonist or antagonist in the absence of said compound(s).

A pharmaceutical composition in unit dosage form, useful in treating a subject afflicted with a neurological disorder, wherein the neurological disorder is selected from the group consisting of Huntington’s disease, Rett syndrome, and CDKL5 disorder, and the other neurological disorders as described herein, which comprises:
(i) an amount of any of the compounds of the present disclosure, or a salt or ester thereof; and
(ii) an amount of an NMDA receptor antagonist, an NMDA receptor partial agonist, a neurokinin 1 receptor antagonist, a neurokinin 2 receptor antagonist, a neurokinin 3 receptor antagonist, or a DOR agonist,
   wherein the respective amounts of said compound(s) and said agonist or antagonist in said composition are effective, upon concomitant administration to said subject of one or more of said unit dosage forms of said composition, to treat the disorder.

A pharmaceutical composition in unit dosage form, useful in treating a subject afflicted with a neurological disorder, wherein the neurological disorder is selected from the group consisting of Huntington’s disease, Rett syndrome, and CDKL5 disorder, and the other neurological disorders as described herein, which comprises:
(i) an amount of any of the compounds of the present disclosure, or a salt or ester thereof; and
(ii) an amount of a DOR antagonist,
   wherein the respective amounts of said compound(s) and said antagonist in said composition are effective, upon concomitant administration to said subject of one or more of said unit dosage forms of said composition, to treat the disorder.

A pharmaceutical composition in unit dosage form, useful in treating a subject afflicted with a neurological disorder, wherein the neurological disorder is selected from the group consisting of Huntington’s disease, Rett syndrome, and CDKL5 disorder, and the other neurological disorders as described herein, which comprises:
(i) an amount of any of the compounds of the present disclosure, or a salt or ester thereof; and
(ii) an amount of an NMDA receptor antagonist, an NMDA receptor partial agonist, a neurokinin 1 receptor antagonist, a DOR agonist or a DOR antagonist,
   wherein the respective amounts of said compound(s) and said agonist or antagonist in said composition are effective, upon concomitant administration to said subject of one or more of said unit dosage forms of said composition, to treat the disorder.

A pharmaceutical composition in unit dosage form, useful in treating a subject afflicted with a neurological disorder, wherein the neurological disorder is selected from the group consisting of Huntington’s disease, Rett syndrome, and CDKL5 disorder, and the other neurological disorders as described herein, which comprises:
(i) an amount of any of the compounds of the present disclosure, or a salt or ester thereof; and
(ii) an amount of a DOR agonist or a DOR antagonist,
   wherein the respective amounts of said compound(s) and said agonist or antagonist in said composition are effective, upon concomitant administration to said subject of one or more of said unit dosage forms of said composition, to treat the disorder.

A pharmaceutical composition in unit dosage form, useful in treating a subject afflicted with a neurological disorder, wherein the neurological disorder is selected from the group consisting of Huntington’s disease, Rett syndrome, and CDKL5 disorder, and the other neurological disorders as described herein, which comprises:
(i) an amount of any of the compounds of the present disclosure, or a salt or ester thereof; and
(ii) an amount of an NMDA receptor antagonist, an NMDA receptor partial agonist, a neurokinin 1 receptor antagonist, or a DOR antagonist,
   wherein the respective amounts of said compound(s) and said agonist or antagonist in said composition are effective, upon concomitant administration to said subject of one or more of said unit dosage forms of said composition, to treat the disorder.

A pharmaceutical composition in unit dosage form, useful in treating a subject afflicted with a neurological disorder, wherein the neurological disorder is selected from the group consisting of Huntington’s disease, Rett syndrome, and CDKL5 disorder, and the other neurological disorders as described herein, which comprises:
(i) an amount of any of the compounds of the present disclosure, or a salt or ester thereof; and
(ii) an amount of a DOR agonist or a DOR antagonist,
   wherein the respective amounts of said compound(s) and said agonist or antagonist in said composition are effective, upon concomitant administration to said subject of one or more of said unit dosage forms of said composition, to treat the disorder.

The pharmaceutical composition of the above embodiment, wherein the respective amounts of said compound(s) and said agonist or antagonist in said unit dose when taken together is more effective to treat the disorder than when compared to the administration of said compound(s) in the absence of said agonist or antagonist or the administration of said agonist or antagonist in the absence of said compound(s).

The present disclosure also provides a method of treating a subject afflicted with a neurological disorder, wherein the neurological disorder is selected from the group consisting of Huntington’s disease, Rett syndrome, and CDKL5 disorder, and the other neurological disorders as described herein comprising administering to the subject an effective amount of an SSRI or an SNRI and an effective amount of any of the compounds of this disclosure, or a salt or ester thereof, so as to thereby treat the disorder.

The present disclosure also provides a compound of this disclosure or a salt or ester thereof, for use as an add-on therapy or in combination with an SSRI or an SNRI in treating a subject afflicted with a neurological disorder, wherein the neurological disorder is selected from the group consisting of Huntington’s disease, Rett syndrome, and CDKL5 disorder, and the other neurological disorders as described herein.

The present disclosure further provides a pharmaceutical composition comprising an amount of any of the of the compounds of this disclosure, or a salt or ester thereof, and an amount of an SSRI or an SNRI for use in treating a subject afflicted with a neurological disorder, wherein the neurological disorder is selected from the group consisting of Huntington’s disease, Rett syndrome, and CDKL5 disorder, and the other neurological disorders as described herein.

The present disclosure also provides a method of treating a subject afflicted with a neurological disorder, wherein the neurological disorder is selected from the group consisting of Huntington’s disease, Rett syndrome, and CDKL5 disorder, and the other neurological disorders as described herein comprising administering to the subject an effective amount of an NMDA receptor antagonist, an NMDA receptor partial agonist, a neurokinin 1 receptor antagonist, a neurokinin 2 receptor antagonist, a neurokinin 3 receptor antagonist, or a DOR agonist and an effective amount of any of the compounds of the present disclosure, or a salt or ester thereof, so as to thereby treat the disorder.

The present disclosure also provides a compound of this disclosure or a salt or ester thereof, for use as an add-on therapy or in combination with an NMDA receptor antagonist, an NMDA receptor partial agonist, a neurokinin 1 receptor antagonist, a neurokinin 2 receptor antagonist, a neurokinin 3 receptor antagonist, or a DOR agonist in treating a subject afflicted with a neurological disorder, wherein the neurological disorder is selected from the group consisting of Huntington’s disease, Rett syndrome, and CDKL5 disorder, and the other neurological disorders as described herein.

The present disclosure further provides a pharmaceutical composition comprising an amount of any of the compounds of this disclosure, or a salt or ester thereof, and an amount of an NMDA receptor antagonist, an NMDA receptor partial agonist, a neurokinin 1 receptor antagonist, a neurokinin 2 receptor antagonist, a neurokinin 3 receptor antagonist, or a DOR agonist for use in treating a subject afflicted with a neurological disorder, wherein the neurological disorder is selected from the group consisting of Huntington’s disease, Rett syndrome, and CDKL5 disorder, and the other neurological disorders as described herein.

The present disclosure also provides a method of treating a subject afflicted with a neurological disorder, wherein the neurological disorder is selected from the group consisting of Huntington’s disease, Rett syndrome, and CDKL5 disorder, and the other neurological disorders as described herein comprising administering to the subject an effective amount of an NMDA receptor antagonist and an effective amount of any of the compounds of the present disclosure, or a salt or ester thereof, so as to thereby treat the disorder.

The present disclosure also provides a compound of this disclosure or a salt or ester thereof, for use as an add-on therapy or in combination with an NMDA receptor antagonist, in treating a subject afflicted with a neurological disorder, wherein the neurological disorder is selected from the group consisting of Huntington’s disease, Rett syndrome, and CDKL5 disorder, and the other neurological disorders as described herein.

The present disclosure further provides a pharmaceutical composition comprising an amount of any of the compounds of this disclosure, or a salt or ester thereof, and an amount of an NMDA receptor antagonist, for use in treating a subject afflicted with a neurological disorder, wherein the neurological disorder is selected from the group consisting of Huntington’s disease, Rett syndrome, and CDKL5 disorder, and the other neurological disorders as described herein.

The present disclosure also provides a method of treating a subject afflicted with a neurological disorder, wherein the neurological disorder is selected from the group consisting of Huntington’s disease, Rett syndrome, and CDKL5 disorder, and the other neurological disorders as described herein comprising administering to the subject an effective amount of an NMDA receptor partial agonist, and an effective amount of any of the compounds of the present disclosure, or a salt or ester thereof, so as to thereby treat the disorder.

The present disclosure also provides a compound of this disclosure or a salt or ester thereof, for use as an add-on therapy or in combination with an NMDA receptor partial agonist in treating a subject afflicted with a neurological disorder, wherein the neurological disorder is selected from the group consisting of Huntington’s disease, Rett syndrome, and CDKL5 disorder, and the other neurological disorders as described herein.

The present disclosure further provides a pharmaceutical composition comprising an amount of any of the compounds of this disclosure, or a salt or ester thereof, and an amount of an NMDA receptor partial agonist, for use in treating a subject afflicted with a neurological disorder, wherein the neurological disorder is selected from the group consisting of Huntington’s disease, Rett syndrome, and CDKL5 disorder, and the other neurological disorders as described herein.

The present disclosure also provides a method of treating a subject afflicted with a neurological disorder, wherein the neurological disorder is selected from the group consisting of Huntington’s disease, Rett syndrome, and CDKL5 disorder, and the other neurological disorders as described herein comprising administering to the subject an effective amount of a neurokinin 1 receptor antagonist and an effective amount of any of the compounds of the present disclosure, or a salt or ester thereof, so as to thereby treat the disorder.

The present disclosure also provides a compound of this disclosure or a salt or ester thereof, for use as an add-on therapy or in combination with a neurokinin 1 receptor antagonist in treating a subject afflicted with a neurological disorder, wherein the neurological disorder is selected from the group consisting of Huntington’s disease, Rett syndrome, and CDKL5 disorder, and the other neurological disorders as described herein.

The present disclosure further provides a pharmaceutical composition comprising an amount of any of the compounds of this disclosure, or a salt or ester thereof, and an amount of a neurokinin 1 receptor antagonist for use in treating a subject afflicted with a neurological disorder, wherein the neurological disorder is selected from the group consisting of Huntington’s disease, Rett syndrome, and CDKL5 disorder, and the other neurological disorders as described herein.

The present disclosure also provides a method of treating a subject afflicted with a neurological disorder, wherein the neurological disorder is selected from the group consisting of Huntington’s disease, Rett syndrome, and CDKL5 disorder, and the other neurological disorders as described herein comprising administering to the subject an effective amount of a neurokinin 2 receptor antagonist and an effective amount of any of the compounds of the present disclosure, or a salt or ester thereof, so as to thereby treat the disorder.

The present disclosure also provides a compound of this disclosure or a salt or ester thereof, for use as an add-on therapy or in combination with a neurokinin 2 receptor antagonist in treating a subject afflicted with a neurological disorder, wherein the neurological disorder is selected from the group consisting of Huntington’s disease, Rett syndrome, and CDKL5 disorder, and the other neurological disorders as described herein.

The present disclosure further provides a pharmaceutical composition comprising an amount of any of the compounds of this disclosure, or a salt or ester thereof, and an amount of a neurokinin 2 receptor antagonist for use in treating a subject afflicted with a neurological disorder, wherein the neurological disorder is selected from the group consisting of Huntington’s disease, Rett syndrome, and CDKL5 disorder, and the other neurological disorders as described herein.

The present disclosure also provides a method of treating a subject afflicted with a neurological disorder, wherein the neurological disorder is selected from the group consisting of Huntington’s disease, Rett syndrome, and CDKL5 disorder, and the other neurological disorders as described herein comprising administering to the subject an effective amount of a neurokinin 3 receptor antagonist and an effective amount of any of the compounds of the present disclosure, or a salt or ester thereof, so as to thereby treat the disorder.

The present disclosure also provides a compound of this disclosure or a salt or ester thereof, for use as an add-on therapy or in combination with a neurokinin 3 receptor antagonist in treating a subject afflicted with a neurological disorder, wherein the neurological disorder is selected from the group consisting of Huntington’s disease, Rett syndrome, and CDKL5 disorder, and the other neurological disorders as described herein.

The present disclosure further provides a pharmaceutical composition comprising an amount of any of the compounds of this disclosure, or a salt or ester thereof, and an amount of a neurokinin 3 receptor antagonist for use in treating a subject afflicted with a neurological disorder, wherein the neurological disorder is selected from the group consisting of Huntington’s disease, Rett syndrome, and CDKL5 disorder, and the other neurological disorders as described herein.

The present disclosure also provides a method of treating a subject afflicted with a neurological disorder, wherein the neurological disorder is selected from the group consisting of Huntington’s disease, Rett syndrome, and CDKL5 disorder, and the other neurological disorders as described herein comprising administering to the subject an effective amount of a DOR agonist and an effective amount of any of the compounds of the present disclosure, or a salt or ester thereof, so as to thereby treat the disorder.

The present disclosure also provides a compound of this disclosure or a salt or ester thereof, for use as an add-on therapy or in combination with a DOR agonist in treating a subject afflicted with a neurological disorder, wherein the neurological disorder is selected from the group consisting of Huntington’s disease, Rett syndrome, and CDKL5 disorder, and the other neurological disorders as described herein.

The present disclosure further provides a pharmaceutical composition comprising an amount of any of the compounds of this disclosure, or a salt or ester thereof, and an amount of a DOR agonist for use in treating a subject afflicted with a neurological disorder, wherein the neurological disorder is selected from the group consisting of Huntington’s disease, Rett syndrome, and CDKL5 disorder, and the other neurological disorders as described herein.

The present disclosure also provides a method of treating a subject afflicted with a neurological disorder, wherein the neurological disorder is selected from the group consisting of Huntington’s disease, Rett syndrome, and CDKL5 disorder, and the other neurological disorders as described herein comprising administering to the subject an effective amount of an NMDA receptor antagonist and an effective amount of any of the compounds of the present disclosure, or a salt or ester thereof, so as to thereby treat the disorder.

The present disclosure also provides a compound of this disclosure or a salt or ester thereof, for use as an add-on therapy or in combination with an NMDA receptor antagonist, in treating a subject afflicted with a neurological disorder, wherein the neurological disorder is selected from the group consisting of Huntington’s disease, Rett syndrome, and CDKL5 disorder, and the other neurological disorders as described herein.

The present disclosure further provides a pharmaceutical composition comprising an amount of any of the compounds of this disclosure, or a salt or ester thereof, and an amount of an NMDA receptor antagonist, for use in treating a subject afflicted with a neurological disorder, wherein the neurological disorder is selected from the group consisting of Huntington’s disease, Rett syndrome, and CDKL5 disorder, and the other neurological disorders as described herein.

The present disclosure also provides a method of treating a subject afflicted with a neurological disorder, wherein the neurological disorder is selected from the group consisting of Huntington’s disease, Rett syndrome, and CDKL5 disorder, and the other neurological disorders as described herein comprising administering to the subject an effective amount of an NMDA receptor partial agonist, and an effective amount of any of the compounds of the present disclosure, or a salt or ester thereof, so as to thereby treat the disorder.

The present disclosure also provides a compound of this disclosure or a salt or ester thereof, for use as an add-on therapy or in combination with an NMDA receptor partial agonist in treating a subject afflicted with a neurological disorder, wherein the neurological disorder is selected from the group consisting of Huntington’s disease, Rett syndrome, and CDKL5 disorder, and the other neurological disorders as described herein.

The present disclosure further provides a pharmaceutical composition comprising an amount of any of the compounds of this disclosure, or a salt or ester thereof, and an amount of an NMDA receptor partial agonist, for use in treating a subject afflicted with a neurological disorder, wherein the neurological disorder is selected from the group consisting of Huntington’s disease, Rett syndrome, and CDKL5 disorder, and the other neurological disorders as described herein.

The present disclosure also provides a method of treating a subject afflicted with a neurological disorder, wherein the neurological disorder is selected from the group consisting of Huntington’s disease, Rett syndrome, and CDKL5 disorder, and the other neurological disorders as described herein comprising administering to the subject an effective amount of a neurokinin 1 receptor antagonist and an effective amount of any of the compounds of the present disclosure, or a salt or ester thereof, so as to thereby treat the disorder.

The present disclosure also provides a compound of this disclosure or a salt or ester thereof, for use as an add-on therapy or in combination with a neurokinin 1 receptor antagonist in treating a subject afflicted with a neurological disorder, wherein the neurological disorder is selected from the group consisting of Huntington’s disease, Rett syndrome, and CDKL5 disorder, and the other neurological disorders as described herein.

The present disclosure further provides a pharmaceutical composition comprising an amount of any of the compounds of this disclosure, or a salt or ester thereof, and an amount of a neurokinin 1 receptor antagonist for use in treating a subject afflicted with a neurological disorder, wherein the neurological disorder is selected from the group consisting of Huntington’s disease, Rett syndrome, and CDKL5 disorder, and the other neurological disorders as described herein.

In some embodiments, a method of treating a subject afflicted with a neurological disorder, wherein the neurological disorder is selected from the group consisting of Huntington’s disease, Rett syndrome, and CDKL5 disorder, and the other neurological disorders as described herein comprising administering to the subject an effective amount of naloxone or methylnaltrexone and an effective amount of a compound of the present disclosure so as to thereby treat the disorder.

In some embodiments, a method of treating a subject afflicted with a neurological disorder, wherein the neurological disorder is selected from the group consisting of Huntington’s disease, Rett syndrome, and CDKL5 disorder, and the other neurological disorders as described herein, comprising administering to the subject an effective amount of naloxone or methylnaltrexone and an effective amount of a compound of the present disclosure so as to thereby treat the disorder.

The present disclosure also provides a method of treating a subject afflicted with a neurological disorder, wherein the neurological disorder is selected from the group consisting of Huntington’s disease, Rett syndrome, and CDKL5 disorder, and the other neurological disorders as described herein comprising administering to the subject an effective amount of a DOR agonist and an effective amount of any of the compounds of the present disclosure, or a salt or ester thereof, so as to thereby treat the disorder.

The present disclosure also provides a compound of this disclosure or a salt or ester thereof, for use as an add-on therapy or in combination with a DOR agonist in treating a subject afflicted with a neurological disorder, wherein the neurological disorder is selected from the group consisting of Huntington’s disease, Rett syndrome, and CDKL5 disorder, and the other neurological disorders as described herein.

The present disclosure further provides a pharmaceutical composition comprising an amount of any of the compounds of this disclosure, or a salt or ester thereof, and an amount of a DOR agonist for use in treating a subject afflicted with a neurological disorder, wherein the neurological disorder is selected from the group consisting of Huntington’s disease, Rett syndrome, and CDKL5 disorder, and the other neurological disorders as described herein.

In any of the embodiments of the present method, compound, package, use or pharmaceutical composition the subject is afflicted with a neurological disorder, wherein the neurological disorder is selected from the group consisting of Huntington’s disease, Rett syndrome, and CDKL5 disorder, and the other neurological disorders as described herein.

In some embodiments of the present method, compound, package, use or pharmaceutical composition, the compound has the structure:

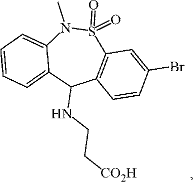

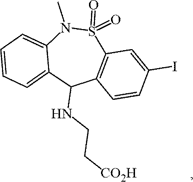

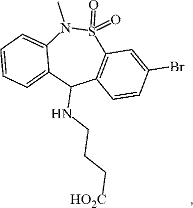

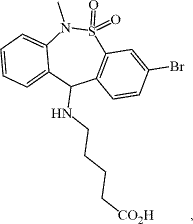

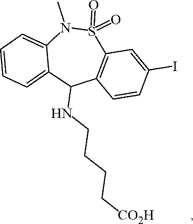

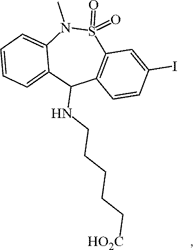

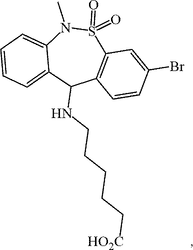

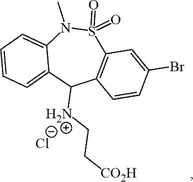

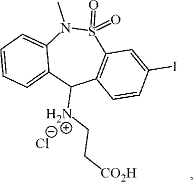

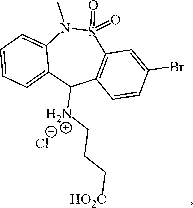

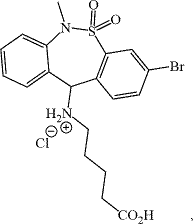

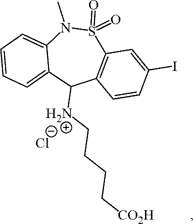

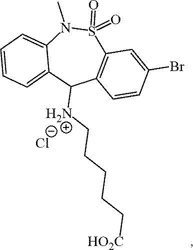

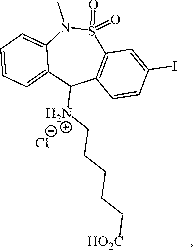

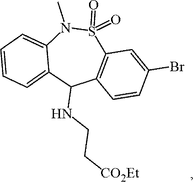

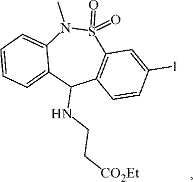

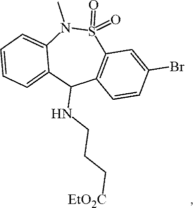

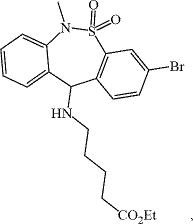

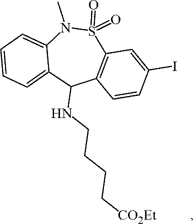

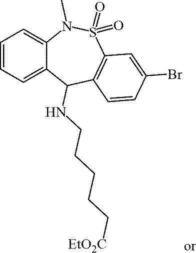

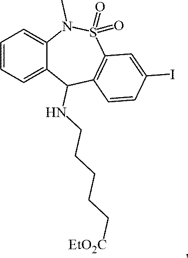

or a pharmaceutically acceptable salt or ester thereof.

In some embodiments of any of the above method, compound, package, use or pharmaceutical composition, the compound has the structure:

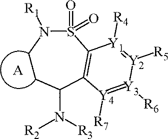

wherein
A is an aryl or heteroaryl, with or without substitution;
R₁ is —H or -(alkyl);
R₂ is -(alkyl), -(alkenyl), -(alkynyl) -(alkyl)-OH, -(alkyl)-CO₂H, -(alkyl)-CO₂-(alkyl), - (alkyl) —C(O) —NH₂, - (alkyl) -C(O) -NH(alkyl), -(alkyl)-C(O)-NH-(hydroxyalkyl), -(alkyl)-C(O)-N(alkyl)₂, -(alkyl)-C(O)-N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-OCH₃, -(alkyl)-(CH)-(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, -NH-(alkyl), -NH-(alkenyl), -NH-(alkynyl), -NH-(aryl), -NH-(heteroaryl), —OH, —OAc, -O-C(O) (alkyl), -O-(alkyl), -O-(alkenyl), -O-(alkynyl), -O-(aryl), -O-(heteroaryl), -S-(alkyl), -S-(alkenyl), -S-(alkynyl), -S-(aryl), -S-(heteroaryl), -S(O)-(alkyl), -S(O)-(aryl), -S(O)-(heteroaryl), -SO₂-(alkyl), -SO₂-(aryl), or -SO2-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
   wherein when A is phenyl, R₁ is —CH₃, R₃, R₄, R₆, and R₇ are each -H, and R₅ is Cl, then R₂ is other than —(CH₂)₄CO₂H, —(CH₂)₆CO₂H, — (CH₂) ₆CO₂CH₂CH₃, or — (CH₂) ₆CH₃,
   wherein when A is phenyl, R₁ is —CH₃, R₃, R₄, R₆, and R₇ are each -H, and R₅ is —SO₂CH₃, then R₂ is other than - (CH₂) ₃OCH₃,
or a pharmaceutically acceptable salt thereof.

In some embodiments of any of the above method, compound, package, use or pharmaceutical composition, the compound has the structure:

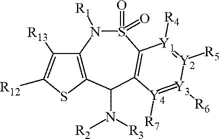

wherein
R₁ is —H or -(alkyl);
R₂ is -(alkyl), -(alkenyl), -(alkynyl), -(alkyl)-OH, -(alkyl)-CO₂H, -(alkyl)-CO₂-(alkyl), - (alkyl) —C(O) —NH₂, - (alkyl) -C(O) -NH(alkyl), -(alkyl)-C(O)-NH-(hydroxyalkyl), -(alkyl)-C(O)-N(alkyl)₂, -(alkyl)-C(O)-N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-OCH₃, -(alkyl)-(CH)-(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, -NH-(alkyl), -NH-(alkenyl), -NH-(alkynyl), -NH-(aryl), -NH-(heteroaryl), —OH, —OAc, -O-C(O) (alkyl), -O-(alkyl), -O-(alkenyl), -O-(alkynyl), -O-(aryl), -O-(heteroaryl), -S-(alkyl), -S-(alkenyl), -S-(alkynyl), -S-(aryl), -S-(heteroaryl), -S(O)-(alkyl), -S(O)-(aryl), -S(O)-(heteroaryl), -SO₂-(alkyl), -SO₂-(aryl), or -SO2-(heteroaryl);
R₁₂ and R₁₃ are each independently —H, —Cl, —Br, —F, —I, —CN, —CF₃, —OCF₃, -(alkyl), -(aryl), -(heteroaryl) -(alkenyl), -(alkynyl), —NH₂, -NH-(alkyl), -NH-(alkenyl), -NH-(alkynyl), -NH-(aryl), -NH-(heteroaryl), —OH, —OAc, -O-C(O) (alkyl), -O-(alkyl), -O-(alkenyl), -O-(alkynyl), -O-(aryl),-O-(heteroaryl), -S-(alkyl), -S-(alkenyl), -S-(alkynyl), -S-(aryl), -S-(heteroaryl), -S(O)-(alkyl), -S(O)-(aryl), -S(O)-(heteroaryl), -SO₂-(alkyl), -SO₂-(aryl), or -SO₂-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
or a pharmaceutically acceptable salt thereof.

In some embodiments of any of the above method, compound, package, use or pharmaceutical composition, the compound has the structure:

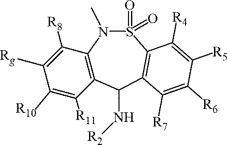

wherein
R₂ is -(alkyl), -(alkenyl), -(alkynyl), -(alkyl)-OH, -(alkyl)-CO₂H, -(alkyl)-CO₂-(alkyl), -(alkyl)-C(O)-NH₂, -(alkyl)-C(O)-NH(alkyl), -(alkyl)-C(O)-NH-(hydroxyalkyl), -(alkyl)-C(O)-N(alkyl)₂, -(alkyl)-C(O)-N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-O-(alkyl), -(alkyl)-(CH)-(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole); R₅ is —Br, or —I;
R₄, R₆ and R₇ are each independently —H, —Cl, —Br, —F, —I, —CN, —CF₃, —OCF₃, -(alkyl), -(alkenyl), -(alkynyl), -(aryl), —NH₂, -NH-(alkyl), -NH-(alkenyl), -NH-(alkynyl), -NH-(aryl), -NH-(heteroaryl), —OH, —OAc, -O-C(O)(alkyl), -O-(alkyl), -O-(alkenyl), -O-(alkynyl), -O-(aryl), -O-(heteroaryl), -S-(alkyl), -S-(alkenyl), -S-(alkynyl), -S-(aryl), -S-(heteroaryl), -S(O)-(alkyl), -S(O)-(aryl), -S(O)-(heteroaryl), -SO₂-(alkyl), -SO₂-(aryl) or -SO2-(heteroaryl); and
R₈, R₉, R₁₀ and R₁₁ are each independently —H, —Cl, —Br, —F, —I, —CN, —CF₃, —OCF₃, -(alkyl), -(aryl), -(heteroaryl) -(alkenyl), -(alkynyl), —NH₂, -NH-(alkyl), -NH-(alkenyl), -NH-(alkynyl), -NH-(aryl), -NH-(heteroaryl), —OH, —OAc, -O-C(O)(alkyl), -O-(alkyl), -O-(alkenyl), -O-(alkynyl), -O-(aryl), -O-(heteroaryl), -S-(alkyl), -S-(alkenyl), -S-(alkynyl), -S-(aryl), -S-(heteroaryl), -S(O)-(alkyl), -S(O)-(aryl), -S(O)-(heteroaryl), -SO₂-(alkyl), -SO₂-(aryl), or -SO₂-(heteroaryl),
or a pharmaceutically acceptable salt thereof.

In some embodiments of any of the above method, compound, package, use or pharmaceutical composition, the compound has the structure:

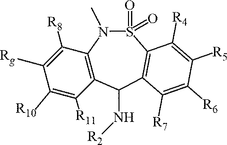

wherein
R₂ is -(alkyl)-O-(alkyl) or -(alkyl)-O-(alkyl)-O-(alkyl);
R₅ is —Cl, —Br, —F, or —I;
R₄, R₆ and R₇ are each independently —H, —Cl, —Br, —F, —I, —CN, —CF₃, —OCF₃, -(alkyl), -(alkenyl), -(alkynyl), -(aryl), —NH₂, —NH—(alkyl), —NH—(alkenyl), —NH—(alkynyl), —NH—(aryl), —NH—(heteroaryl), —OH, —OAc, —O—C(O)(alkyl), —O—(alkyl), —O—(alkenyl), —O—(alkynyl), —O—(aryl), —O—(heteroaryl), —S—(alkyl), —S—(alkenyl), —S—(alkynyl), —S—(aryl), —S—(heteroaryl), —S(O)—(alkyl), —S(O)—(aryl), —S(O)—(heteroaryl), —SO₂—(alkyl), —SO₂—(aryl) or —SO2—(heteroaryl);
R₈, R₉, R₁₀ and R₁₁ are each independently —H, —Cl, —Br, —F, —I, —CN, —CF₃, —OCF₃, -(alkyl), -(aryl), -(heteroaryl) -(alkenyl), -(alkynyl), —NH₂, —NH—(alkyl), —NH—(alkenyl), —NH—(alkynyl), —NH—(aryl), —NH—(heteroaryl), —OH, —OAc, —O—C(O) (alkyl), —O—(alkyl), —O—(alkenyl), —O—(alkynyl), —O—(aryl), —O—(heteroaryl), —S—(alkyl), —S—(alkenyl), —S—(alkynyl), —S—(aryl), —S—(heteroaryl), —S(O)—(alkyl), —S(O)—(aryl), —S(O)—(heteroaryl), —SO₂—(alkyl), —SO₂—(aryl), or —SO₂—(heteroaryl),
or a pharmaceutically acceptable salt thereof.

In some embodiments of any of the above method, compound, package, use or pharmaceutical composition, the compound has the structure:

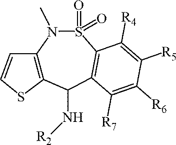

wherein
R₂ is -(alkyl), -(alkenyl), -(alkynyl), -(alkyl)-OH, -(alkyl)-CO₂H, -(alkyl)-CO₂-(alkyl), -(alkyl)—C(O)—NH₂, -(alkyl)-C(O)-NH(alkyl), -(alkyl)—C(O)—NH—(hydroxyalkyl), -(alkyl)-C(O)-N(alkyl)₂, -(alkyl)-C(O)-N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-O-(alkyl), -(alkyl)—(CH)—(O—(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole);
R₅ is —Cl, —Br, —F, or —I;
R₄, R₆ and R₇ are each independently —H, —Cl, —Br, —F, —I, —CN, —CF₃, —OCF₃, -(alkyl), -(alkenyl), -(alkynyl), -(aryl), —NH₂, -NH-(alkyl), —NH—(alkenyl), —NH—(alkynyl), -NH-(aryl), -NH-(heteroaryl), —OH, —OAc, —O—C(O) (alkyl), -O-(alkyl), —O—(alkenyl), -O-(alkynyl), -O-(aryl), -O-(heteroaryl), —S—(alkyl), -S-(alkenyl), -S-(alkynyl), -S-(aryl), —S—(heteroaryl), -S(O)-(alkyl), -S(O)-(aryl), -S(O)-(heteroaryl), -SO₂-(alkyl), -SO₂-(aryl) or -SO2-(heteroaryl),
or a pharmaceutically acceptable salt thereof.

In some embodiments of any of the above method, compound, package, use or pharmaceutical composition, the compound has the structure:

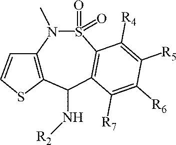

wherein
R₂ is -(alkyl)-CO₂-(alkyl), -(alkyl)-O-(alkyl) or -(alkyl)-O-(alkyl)-O-(alkyl);
R₅ is —Cl, —Br, —F, or —I;
R₄, R₆ and R₇ are each independently —H, —Cl, —Br, —F, —I, —CN, —CF₃, —OCF₃, -(alkyl), -(alkenyl), -(alkynyl), -(aryl), —NH₂, -NH-(alkyl), -NH-(alkenyl), -NH-(alkynyl), -NH-(aryl), —NH—(heteroaryl), —OH, —OAc, -O-C(O)(alkyl), -O-(alkyl), —O—(alkenyl), -O-(alkynyl), -O-(aryl), -O-(heteroaryl), -S-(alkyl), -S-(alkenyl), -S-(alkynyl), -S-(aryl), -S-(heteroaryl), -S(O)-(alkyl), -S(O)-(aryl), -S(O)-(heteroaryl), -SO₂-(alkyl), -SO₂-(aryl) or -SO2-(heteroaryl);
R₈, R₉, R₁₀ and R₁₁ are each independently —H, —Cl, —Br, —F, —I, —CN, —CF₃, —OCF₃, -(alkyl), -(aryl), -(heteroaryl) -(alkenyl), -(alkynyl), —NH₂, -NH-(alkyl), -NH-(alkenyl), -NH-(alkynyl), -NH-(aryl), -NH-(heteroaryl), —OH, —OAc, -O-C(O)(alkyl), —O—(alkyl), -O-(alkenyl), -O-(alkynyl), -O-(aryl), -O-(heteroaryl), -S-(alkyl), -S-(alkenyl), -S-(alkynyl), -S-(aryl), -S-(heteroaryl), -S(O)-(alkyl), -S(O)-(aryl), -S(O)-(heteroaryl), -SO₂-(alkyl), -SO₂-(aryl), or -SO₂-(heteroaryl),
or a pharmaceutically acceptable salt thereof.

In some embodiments of any of the above method, compound, package, use or pharmaceutical composition, the compound has the structure:

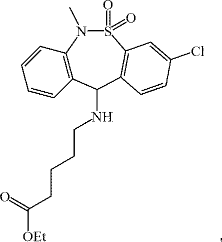

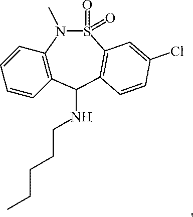

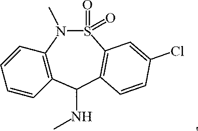

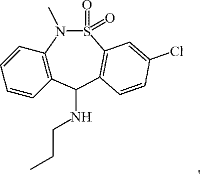

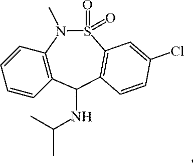

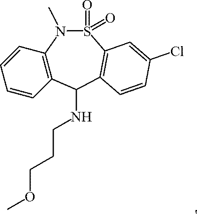

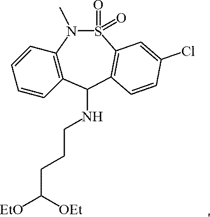

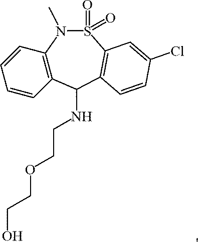

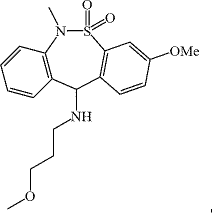

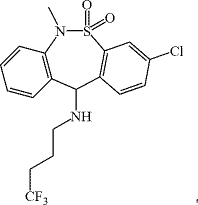

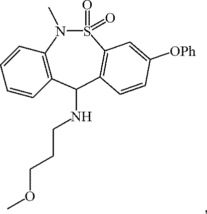

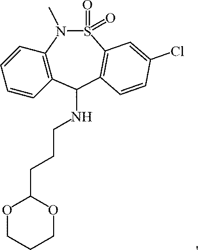

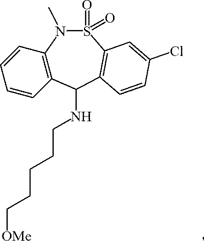

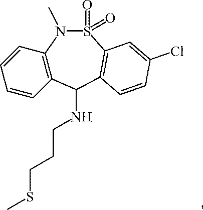

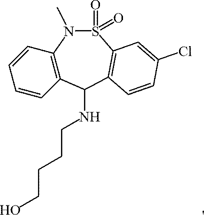

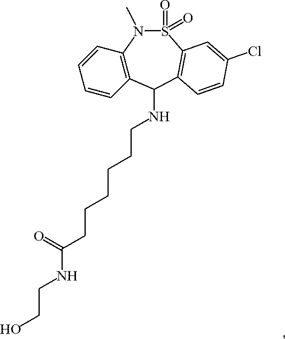

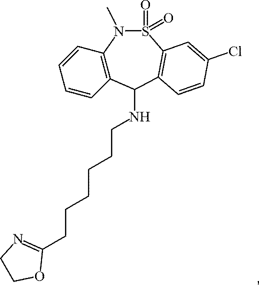

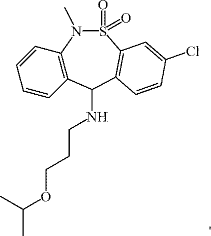

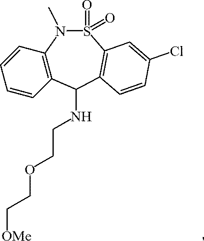

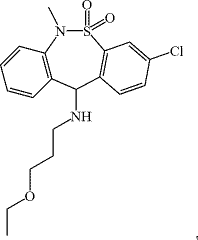

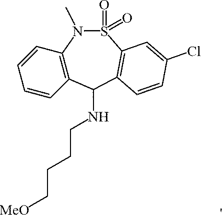

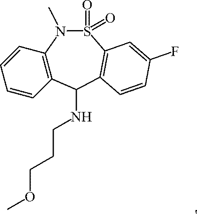

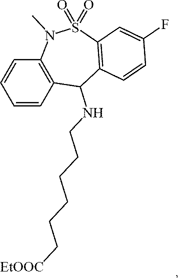

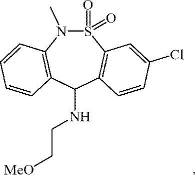

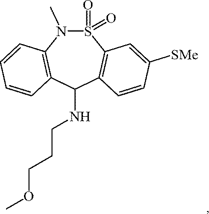

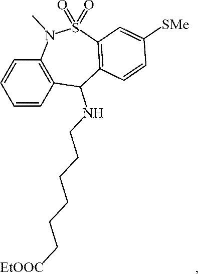

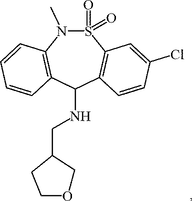

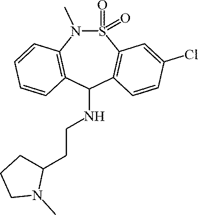 [SMILES: CN1CCCC1CCNC1c2ccccc2N(C)S(=O)(=O)c2cc(Cl)ccc21]

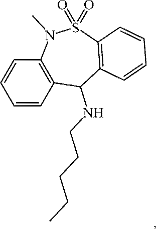

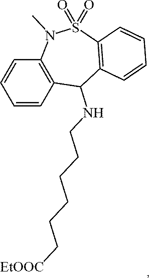

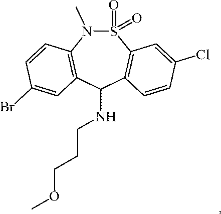

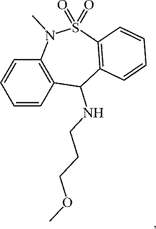

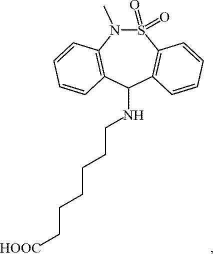

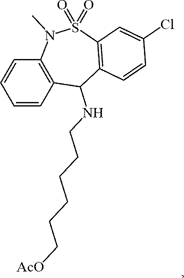

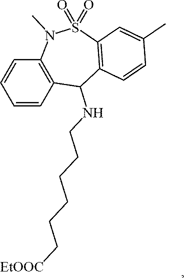

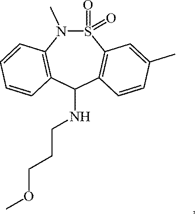

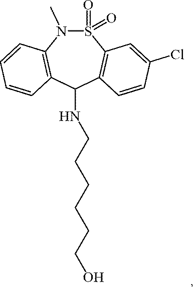

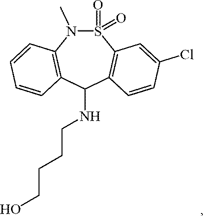

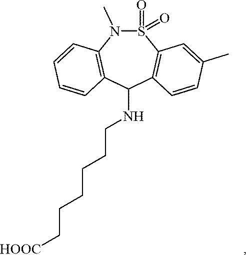

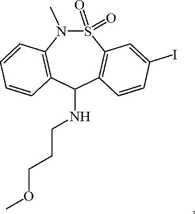

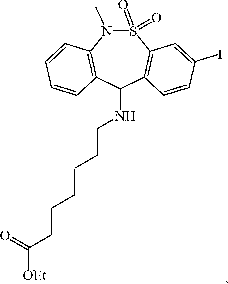

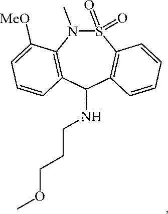

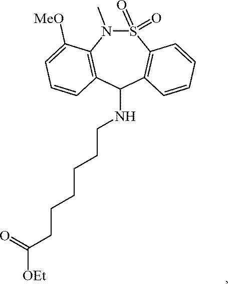

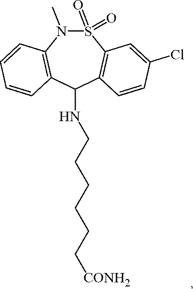

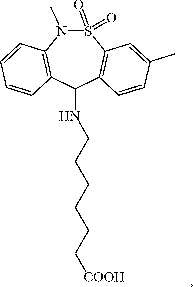

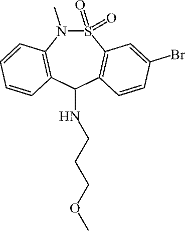 [SMILES: COCCCNC1c2ccccc2N(C)S(=O)(=O)c2cc(Br)ccc21]

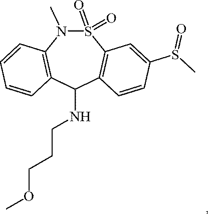

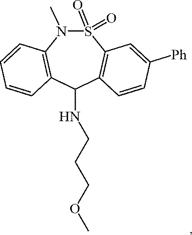

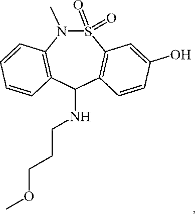

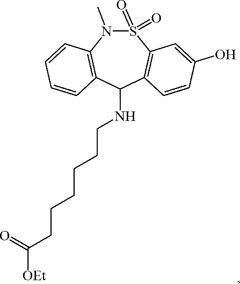

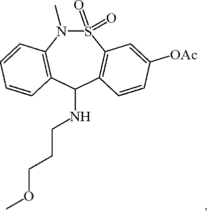

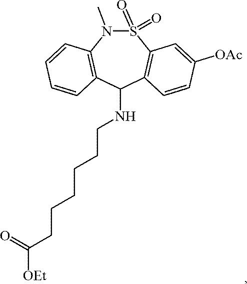

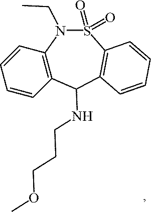

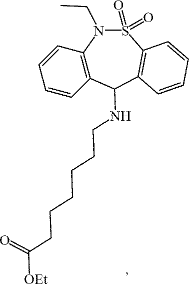

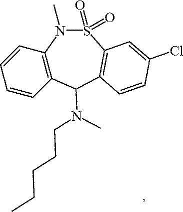

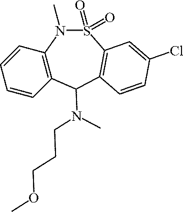 [SMILES: COCCCN(C)C1c2ccccc2N(C)S(=O)(=O)c2cc(Cl)ccc21]

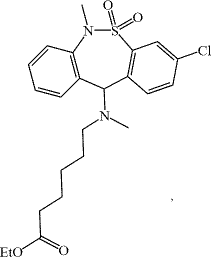

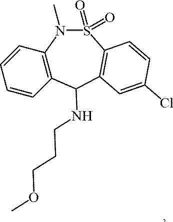

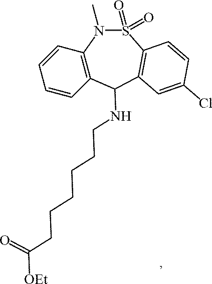

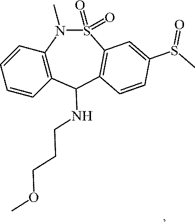

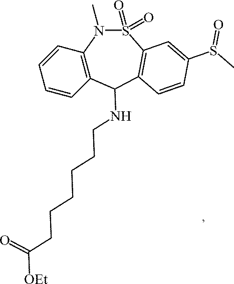

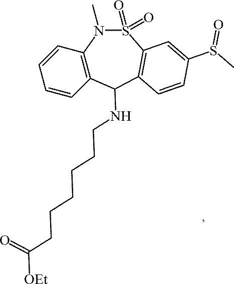

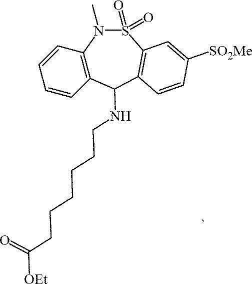

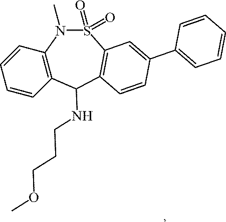

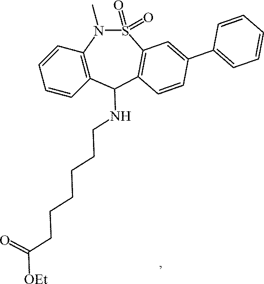

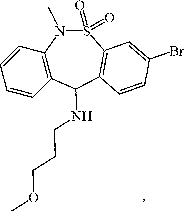

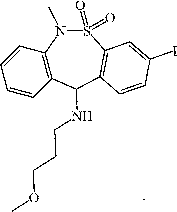

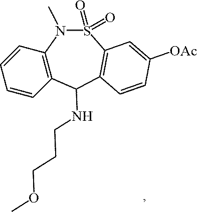

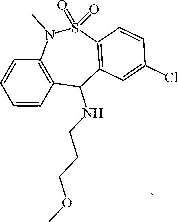

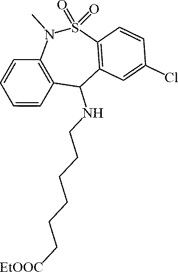 [SMILES: CCOC(=O)CCCCCCNC1c2ccccc2N(C)S(=O)(=O)c2ccc(Cl)cc21]

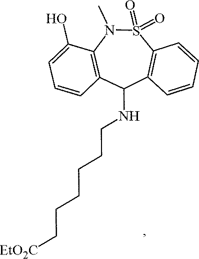

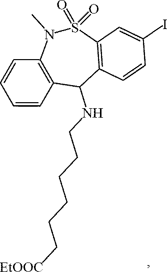

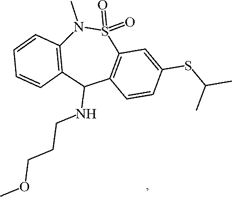

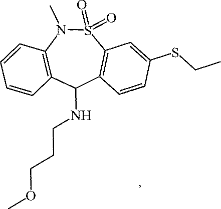

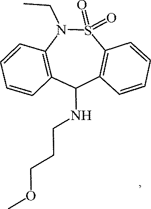

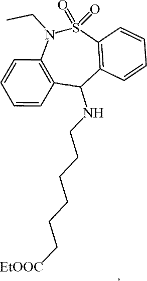

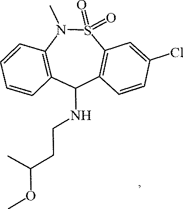

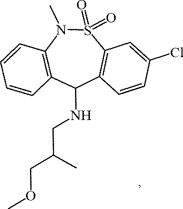

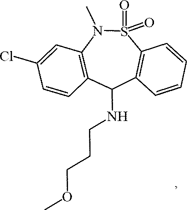

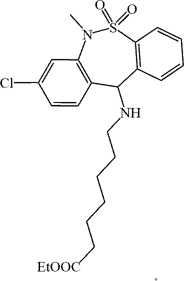

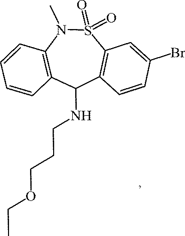

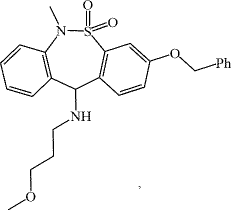

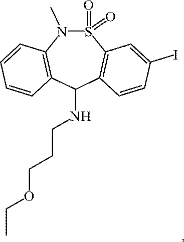

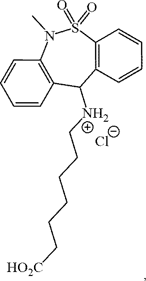

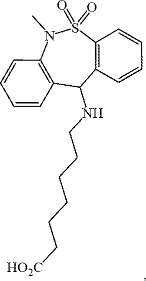

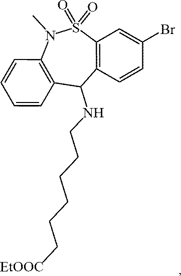

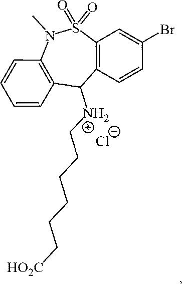

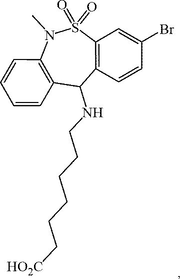

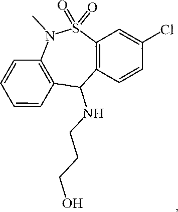

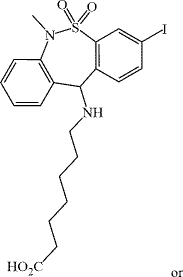

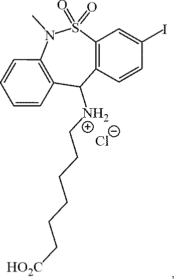

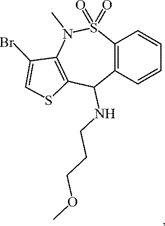

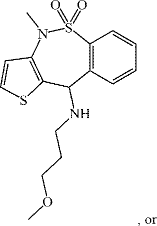

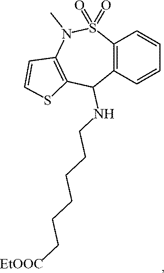

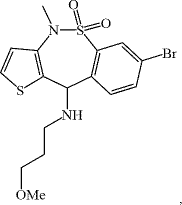

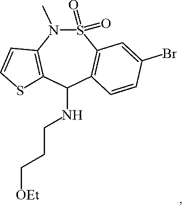

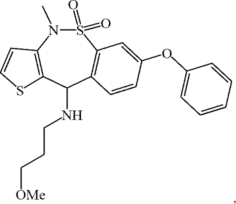

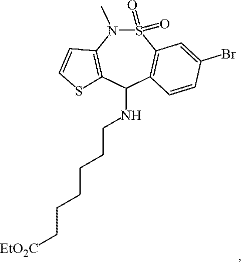

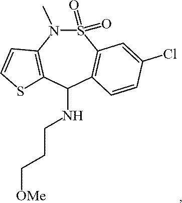

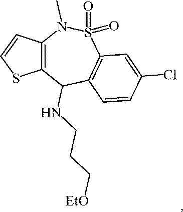

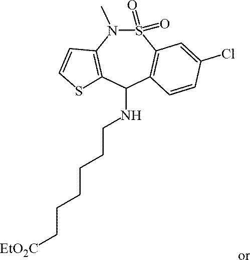

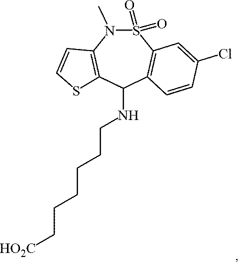

or a pharmaceutically acceptable salt thereof.

In some embodiments, a pharmaceutically acceptable salt of any of the above compounds is used according to the present disclosure.

In some embodiments, a salt of a compound of the present disclosure is used in any of the above methods, uses, packages or compositions.

In some embodiments, a pharmaceutically salt of a compound of the present disclosure is used in any of the above methods, uses, packages or compositions.

In some embodiments, an ester of a compound of the present disclosure is used in any of the above methods, uses, packages or compositions.

Any of the above compounds may be used in any of the disclosed methods, uses, packages or pharmaceutical compositions.

Any of the compounds used in the disclosed methods, uses, packages or pharmaceutical compositions may be replaced with any other compound disclosed herein. Any combination of the disclosed compounds may be used.

Any of the above generic compounds may be used in any of the disclosed methods, uses, packages or compositions.

In some embodiments, the methods, uses, packages or pharmaceutical compositions of the present disclosure wherein the neurological disorder includes, but is not limited to, Huntington’s disease; Rett syndrome; a Rett syndrome variant; Angelman syndrome; Prader-Willi syndrome; neonatal onset encephalopathy; X-linked recessive mental retardation; fetal alcohol spectrum disorder; Hirschsprung disease; CDKL5 disorder; West syndrome; FOXG1 syndrome; and Lennox-Gastaut syndrome.

In some embodiments, the methods, uses, packages or pharmaceutical compositions of the present disclosure wherein the neurological disorder is Huntington’s disease.

In some embodiments, the methods, uses, packages or pharmaceutical compositions of the present disclosure wherein the neurological disorder is Rett syndrome.

In some embodiments, the methods, uses, packages or pharmaceutical compositions of the present disclosure wherein the neurological disorder is CDKL5 disorder.

In some embodiments, the methods, uses, packages or pharmaceutical compositions wherein the symptoms of the neurological disorder include, but are not limited to, involuntary movements; abnormal muscle rigidity or contraction; abnormal hand movements ; poor muscle tone; unusual eye movements; unusual facial expressions; tremors; impaired gait, posture, or balance; frequent falls; loss of balance; difficulty with swallowing; teeth grinding; fatigue; and insomnia.

In some embodiments, the NMDA receptor antagonist is an arylcyclohexylamine, dextromorphinan or adamantane.

In some embodiments, the NMDA receptor antagonist is dextromethorphan, dextrorphan, dextrallorphan, memantine, amantadine, rimantadine, nitromemantine (YQW-36), ketamine (and its analogs, e.g., tiletamine), phencyclidine (and its analogs, e.g., tenocyclidine, eticyclidine, rolicyclidine), methoxetamine (and its analogs), gacyclidine (GK-11), neramexane, lanicemine (AZD6765), diphenidine, dizocilpine (MK-801), 8a-phenyldecahydroquinoline (8A-PDHQ), remacemide, ifenprodil, traxoprodil (CP-101,606), eliprodil (SL-82.0715), etoxadrol (CL-1848C), dexoxadrol, WMS-2539, NEFA, delucemine (NPS-1506), aptiganel (Cerestat; CNS-1102), midafotel (CPPene; SDZ EAA 494), dexanabinol (HU-211 or ETS2101), selfotel (CGS-19755), 7-chlorokynurenic acid (7-CKA), 5,7-dichlorokynurenic acid (5,7-DCKA), L-683344, L-689560, L-701324, GV150526A, GV196771A, CERC-301 (formerly MK-0657), atomoxetine, LY-235959, CGP 61594, CGP 37849, CGP 40116 (active enantiomer of CG 37849), LY-233536, PEAQX (NVP-AAM077), ibogaine, noribogaine, Ro 25-6981, GW468816, EVT-101, indantadol, perzinfotel (EAA-090), SSR240600, 2-MDP (U-23807A) or AP-7.

In some embodiments, the NMDA receptor partial agonist is a NRX-1074 or rapastinel (GLYX-13).

In some embodiments, the neurokinin 1 receptor antagonist is aprepitant, fosaprepitant, casopitant, maropitant, vestipitant, vofopitant, lanepitant, orvepitant, ezlopitant, netupitant, rolapitant, L-733060, L-703606, L-759274, L-822429, L-760735, L-741671, L-742694, L-732138, CP-122721, RPR-100893, CP-96345, CP-99994, TAK-637, T-2328, CJ-11974, RP 67580, NKP608, VPD-737, GR 205171, LY686017, AV608, SR140333B, SSR240600C, FK 888 or GR 82334.

In some embodiments, the neurokinin 2 receptor antagonist is saredutant, ibodutant, nepadutant, GR-159897 or MEN-10376.

In some embodiments, the neurokinin 3 receptor antagonist is osanetant, talnetant, SB-222200 or SB-218795.

In some embodiments, the DOR agonist is tianeptine, (+)BW373U86, SNC-80, SNC-121, SNC-162, DPI-287, DPI-3290, DPI-221, TAN-67, KN-127, AZD2327, JNJ-20788560, NIH11082, RWJ-394674, ADL5747, ADL5859, UFP-512, AR-M100390, SB-235863 or 7-spiroindanyloxymorphone.

In some embodiments, the DOR antagonist is naltrindole, naltriben, N-benzylnaltrindole, 7-benzylidenenaltrexone, SDM25N, or DPI-2505.

The term “MOR agonist” is intended to mean any compound or substance that activates the mu-opioid receptor (MOR). The agonist may be a partial, full or super agonist.

The term “DOR agonist” is intended to mean any compound or substance that activates the delta-opioid receptor (DOR). The agonist may be a partial, full or super agonist.

The term “DOR antagonist” is intended to mean any compound or substance that blocks or reverses activation of the delta-opioid receptor (DOR).

In some embodiments, the compound is prepared by the following process:

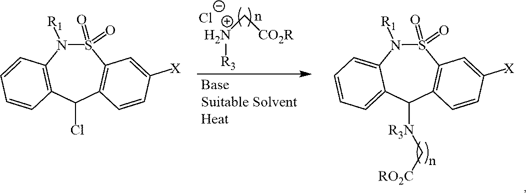

wherein R is -(alkyl); R₁ is —H or -(alkyl); R₃ is —H or -(alkyl); X = Br or I; and n = 2-5.

In some embodiments, the compound is prepared by the following process:

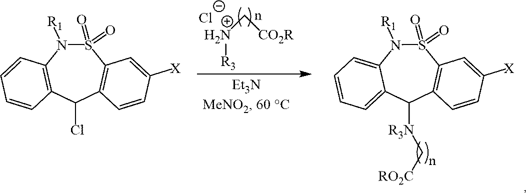

wherein R is -(alkyl); R₁ is —H or -(alkyl); R₃ is —H or -(alkyl); X = Br or I; and n = 2-5.

In some embodiments, the compound is prepared by the following process:

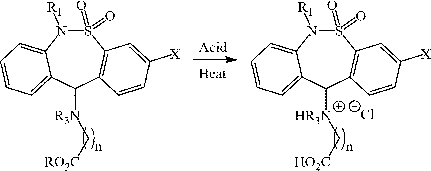

wherein R is -(alkyl); R₁ is —H or -(alkyl); R₃ is —H or -(alkyl); X = Br or I; and n = 2-5.

In one embodiments of the above process, the acid is aqueous acid.

In some embodiments, the compound is prepared by the following process:

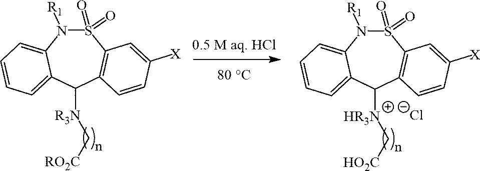

wherein R is -(alkyl); R₁ is —H or -(alkyl); R₃ is —H or -(alkyl); X = Br or I; and n = 2-5.

Except where otherwise specified, the structure of a compound of this disclosure includes an asymmetric carbon atom, it is understood that the compound occurs as a racemate, racemic mixture, and isolated single enantiomer. All such isomeric forms of these compounds are expressly included in this disclosure. Except where otherwise specified, each stereogenic carbon may be of the R or S configuration. It is to be understood accordingly that the isomers arising from such asymmetry (e.g., all enantiomers and diastereomers) are included within the scope of this disclosure, unless indicated otherwise. Such isomers can be obtained in substantially pure form by classical separation techniques and by stereochemically controlled synthesis, such as those described in “Enantiomers, Racemates and Resolutions” by J. Jacques, A. Collet and S. Wilen, Pub. John Wiley & Sons, NY, 1981. For example, the resolution may be carried out by preparative chromatography on a chiral column.

The present disclosure is also intended to include all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include C-13 and C-14.

It will be noted that any notation of a carbon in structures throughout this application, when used without further notation, are intended to represent all isotopes of carbon, such as ¹²C, ¹³C, or ¹⁴C. Furthermore, any compounds containing ¹³C or ¹⁴C may specifically have the structure of any of the compounds disclosed herein.

It will also be noted that any notation of a hydrogen in structures throughout this application, when used without further notation, are intended to represent all isotopes of hydrogen, such as ¹H, ²H, or ³H. Furthermore, any compounds containing ²H or ³H may specifically have the structure of any of the compounds disclosed herein.

Isotopically-labeled compounds can generally be prepared by conventional techniques known to those skilled in the art using appropriate isotopically-labeled reagents in place of the non-labeled reagents employed.

In the compounds used in the method of the present disclosure, the substituents may be substituted or unsubstituted, unless specifically defined otherwise.

In the compounds used in the method of the present disclosure, alkyl, heteroalkyl, monocycle, bicycle, aryl, heteroaryl and heterocycle groups can be further substituted by replacing one or more hydrogen atoms with alternative non-hydrogen groups. These include, but are not limited to, halo, hydroxy, mercapto, amino, carboxy, cyano and carbamoyl.

It is understood that substituents and substitution patterns on the compounds used in the method of the present disclosure can be selected by one of ordinary skill in the art to provide compounds that are chemically stable and that can be readily synthesized by techniques known in the art from readily available starting materials. If a substituent is itself substituted with more than one group, it is understood that these multiple groups may be on the same carbon or on different carbons, so long as a stable structure results.

In choosing the compounds used in the method of the present disclosure, one of ordinary skill in the art will recognize that the various substituents, i.e., R₁, R₂, etc. are to be chosen in conformity with well-known principles of chemical structure connectivity.

As used herein, “alkyl” is intended to include both branched and straight-chain saturated aliphatic hydrocarbon groups having the specified number of carbon atoms. Thus, C₁-Cₙ as in “C₁-Cₙ alkyl” is defined to include groups having 1, 2......, n-1 or n carbons in a linear or branched arrangement, and specifically includes methyl, ethyl, propyl, butyl, pentyl, hexyl, heptyl, isopropyl, isobutyl, sec-butyl and so on. An embodiment can be C₁-C₁₂ alkyl, C₂-C₁₂ alkyl, C₃-C₁₂ alkyl, C₄-C₁₂ alkyl and so on. An embodiment can be C₁-C₈ alkyl, C₂-C₈ alkyl, C₃-C₈ alkyl, C₄-C₈ alkyl and so on. “Alkoxy” represents an alkyl group as described above attached through an oxygen bridge.

The term “alkenyl” refers to a non-aromatic hydrocarbon radical, straight or branched, containing at least 1 carbon to carbon double bond, and up to the maximum possible number of non-aromatic carbon-carbon double bonds may be present. Thus, C₂-Cₙ alkenyl is defined to include groups having 1, 2...., n-1 or n carbons. For example, “C₂-C₆ alkenyl” means an alkenyl radical having 2, 3, 4, 5, or 6 carbon atoms, and at least 1 carbon-carbon double bond, and up to, for example, 3 carbon-carbon double bonds in the case of a C₆ alkenyl, respectively. Alkenyl groups include ethenyl, propenyl, butenyl and cyclohexenyl. As described above with respect to alkyl, the straight, branched or cyclic portion of the alkenyl group may contain double bonds and may be substituted if a substituted alkenyl group is indicated. An embodiment can be C₂-C₁₂ alkenyl or C₂-C₈ alkenyl.

The term “alkynyl” refers to a hydrocarbon radical straight or branched, containing at least 1 carbon to carbon triple bond, and up to the maximum possible number of non-aromatic carbon-carbon triple bonds may be present. Thus, C₂-Cₙ alkynyl is defined to include groups having 1, 2...., n-1 or n carbons. For example, “C₂-C₆ alkynyl” means an alkynyl radical having 2 or 3 carbon atoms, and 1 carbon-carbon triple bond, or having 4 or 5 carbon atoms, and up to 2 carbon-carbon triple bonds, or having 6 carbon atoms, and up to 3 carbon-carbon triple bonds. Alkynyl groups include ethynyl, propynyl and butynyl. As described above with respect to alkyl, the straight or branched portion of the alkynyl group may contain triple bonds and may be substituted if a substituted alkynyl group is indicated. An embodiment can be a C₂-Cₙ alkynyl. An embodiment can be C₂-C₁₂ alkynyl or C₃-C₈ alkynyl.

As used herein, “hydroxyalkyl” includes alkyl groups as described above wherein one or more bonds to hydrogen contained therein are replaced by a bond to an —OH group. In some embodiments, C₁-C₁₂ hydroxyalkyl or C₁-C₆ hydroxyalkyl. C₁-Cₙ as in “C₁-Cₙalkyl” is defined to include groups having 1, 2, ...., n-1 or n carbons in a linear or branched arrangement (e.g., C₁-C₂ hydroxyalkyl, C₁-C₃ hydroxyalkyl, C₁-C₄ hydroxyalkyl, C₁-C₅hydroxyalkyl, or C₁-C₆ hydroxyalkyl) For example, C₁-C₆, as in “C₁-C₆ hydroxyalkyl” is defined to include groups having 1, 2, 3, 4, 5, or 6 carbons in a linear or branched alkyl arrangement wherein a hydrogen contained therein is replaced by a bond to an -OH group.

As used herein, “heteroalkyl” includes both branched and straight-chain saturated aliphatic hydrocarbon groups having the specified number of carbon atoms and at least 1 heteroatom within the chain or branch.

As used herein, “monocycle” includes any stable polyatomic carbon ring of up to 10 atoms and may be unsubstituted or substituted. Examples of such non-aromatic monocycle elements include but are not limited to: cyclobutyl, cyclopentyl, cyclohexyl, and cycloheptyl. Examples of such aromatic monocycle elements include but are not limited to: phenyl.

As used herein, “bicycle” includes any stable polyatomic carbon ring of up to 10 atoms that is fused to a polyatomic carbon ring of up to 10 atoms with each ring being independently unsubstituted or substituted. Examples of such non-aromatic bicycle elements include but are not limited to: decahydronaphthalene. Examples of such aromatic bicycle elements include but are not limited to: naphthalene.

As used herein, “aryl” is intended to mean any stable monocyclic, bicyclic or polycyclic carbon ring of up to 10 atoms in each ring, wherein at least one ring is aromatic, and may be unsubstituted or substituted. Examples of such aryl elements include but are not limited to: phenyl, p-toluenyl (4-methylphenyl), naphthyl, tetrahydro-naphthyl, indanyl, phenanthryl, anthryl or acenaphthyl. In cases where the aryl substituent is bicyclic and one ring is non-aromatic, it is understood that attachment is via the aromatic ring.

The term “heteroaryl”, as used herein, represents a stable monocyclic, bicyclic or polycyclic ring of up to 10 atoms in each ring, wherein at least one ring is aromatic and contains from 1 to 4 heteroatoms selected from the group consisting of O, N and S. Bicyclic aromatic heteroaryl groups include phenyl, pyridine, pyrimidine or pyridazine rings that are (a) fused to a 6-membered aromatic (unsaturated) heterocyclic ring having one nitrogen atom; (b) fused to a 5- or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

The term “heterocycle”, “heterocyclyl” or “heterocyclic” refers to a mono- or poly-cyclic ring system which can be saturated or contains one or more degrees of unsaturation and contains one or more heteroatoms. Preferred heteroatoms include N, O, and/or S, including N-oxides, sulfur oxides, and dioxides. Preferably the ring is three to ten-membered and is either saturated or has one or more degrees of unsaturation. The heterocycle may be unsubstituted or substituted, with multiple degrees of substitution being allowed. Such rings may be optionally fused to one or more of another “heterocyclic” ring(s), heteroaryl ring(s), aryl ring(s), or cycloalkyl ring(s). Examples of heterocycles include, but are not limited to, tetrahydrofuran, pyran, 1,4-dioxane, 1,3-dioxane, piperidine, piperazine, pyrrolidine, morpholine, thiomorpholine, tetrahydrothiopyran, tetrahydrothiophene, 1,3-oxathiolane, and the like.

The term “ester” is intended to a mean an organic compound containing the R-O-CO-R′ group.

The term “substitution”, “substituted” and “substituent” refers to a functional group as described above in which one or more bonds to a hydrogen atom contained therein are replaced by a bond to non-hydrogen or non-carbon atoms, provided that normal valencies are maintained and that the substitution results in a stable compound. Substituted groups also include groups in which one or more bonds to a carbon(s) or hydrogen(s) atom are replaced by one or more bonds, including double or triple bonds, to a heteroatom. Examples of substituent groups include the functional groups described above, and halogens (i.e., F, Cl, Br, and I); alkyl groups, such as methyl, ethyl, n-propyl, isopropryl, n-butyl, tert-butyl, and trifluoromethyl; hydroxyl; alkoxy groups, such as methoxy, ethoxy, n-propoxy, and isopropoxy; aryloxy groups, such as phenoxy; arylalkyloxy, such as benzyloxy (phenylmethoxy) and p-trifluoromethylbenzyloxy (4-trifluoromethylphenylmethoxy); heteroaryloxy groups; sulfonyl groups, such as trifluoromethanesulfonyl, methanesulfonyl, and p-toluenesulfonyl; nitro, nitrosyl; mercapto; sulfanyl groups, such as methylsulfanyl, ethylsulfanyl and propylsulfanyl; cyano; amino groups, such as amino, methylamino, dimethylamino, ethylamino, and diethylamino; and carboxyl. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

The compounds used in the method of the present disclosure may be prepared by techniques well known in organic synthesis and familiar to a practitioner ordinarily skilled in the art. However, these may not be the only means by which to synthesize or obtain the desired compounds.

The compounds used in the method of the present disclosure may be prepared by techniques described in Vogel’s Textbook of Practical Organic Chemistry, A.I. Vogel, A.R. Tatchell, B.S. Furnis, A.J. Hannaford, P.W.G. Smith, (Prentice Hall) 5^{th} Edition (1996), March’s Advanced Organic Chemistry: Reactions, Mechanisms, and Structure, Michael B. Smith, Jerry March, (Wiley-Interscience) 5^{th} Edition (2007), and references therein, which are incorporated by reference herein. However, these may not be the only means by which to synthesize or obtain the desired compounds.

The various R groups attached to the aromatic rings of the compounds disclosed herein may be added to the rings by standard procedures, for example those set forth in Advanced Organic Chemistry: Part B: Reactions and Synthesis, Francis Carey and Richard Sundberg, (Springer) 5th ed. Edition. (2007), the content of which is hereby incorporated by reference.

Another aspect of the disclosure comprises a compound used in the method of this disclosure as a pharmaceutical composition.

As used herein, the term “pharmaceutically active agent” means any substance or compound suitable for administration to a subject and furnishes biological activity or other direct effect in the treatment, cure, mitigation, diagnosis, or prevention of disease, or affects the structure or any function of the subject. Pharmaceutically active agents include, but are not limited to, substances and compounds described in the Physicians’ Desk Reference (PDR Network, LLC; 64th edition; Nov. 15, 2009) and “Approved Drug Products with Therapeutic Equivalence Evaluations” (U.S. Department of Health and Human Services, 30^{th} edition, 2010), which are hereby incorporated by reference. Pharmaceutically active agents which have pendant carboxylic acid groups may be modified in accordance with the present disclosure using standard esterification reactions and methods readily available and known to those having ordinary skill in the art of chemical synthesis. Where a pharmaceutically active agent does not possess a carboxylic acid group, the ordinarily skilled artisan will be able to design and incorporate a carboxylic acid group into the pharmaceutically active agent where esterification may subsequently be carried out so long as the modification does not interfere with the pharmaceutically active agent’s biological activity or effect.

The compounds used in the method of the present disclosure may be in a salt form. As used herein, a “salt” is a salt of the disclosed compounds which has been modified by making acid or base salts of the compounds. In the case of compounds used to treat an infection or disease caused by a pathogen, the salt is pharmaceutically acceptable. Examples of pharmaceutically acceptable salts include, but are not limited to, mineral or organic acid salts of basic residues such as amines; alkali or organic salts of acidic residues such as phenols; alkali or organic salts of acidic residues such as carboxylic acids. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkali earth metal salts, sodium, potassium or lithium. The salts can be made using an organic or inorganic base. Such basic salts are alkali metal salts, such as sodium, potassium or lithium and alkaline earth metal salts, such as magnesium and calcium.

The term “pharmaceutically acceptable salt” in this respect, refers to the relatively non-toxic, inorganic and organic acid or base addition salts of compounds of the present disclosure. These salts can be prepared in situ during the final isolation and purification of the compounds of the disclosure, or by separately reacting a purified compound of the disclosure in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. Representative salts also include the sodium, potassium, lithium, magnesium and calcium salts and the like. (See, e.g., Berge et al. (1977) “Pharmaceutical Salts”, J. Pharm. Sci. 66:1-19).

Administration of one or more compounds and/or one or more compositions (e.g., pharmaceutical compositions) disclosed herein may be used for preventing, slowing, halting, or reversing the progression of a neurological disorder, as set out herein. Administration may also be used for improving one or more symptoms of the neurological disorder.

The compounds used in the method of the present disclosure may be administered in various forms, including those detailed herein. The treatment with a compound may be a component of a combination therapy or an adjunct therapy, i.e., the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

As used herein, a “pharmaceutically acceptable carrier” is a pharmaceutically acceptable solvent, suspending agent or vehicle, for delivering the instant compounds to the animal or human. The carrier may be liquid or solid and is selected with the planned manner of administration in mind. Liposomes are also a pharmaceutically acceptable carrier.

The dosage of the compounds administered in treatment will vary depending upon factors such as the pharmacodynamic characteristics of a specific therapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds used in the method of the present disclosure may comprise a single compound or mixtures thereof with additional antibacterial agents. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g., by injection, topical application, or other methods, into or onto a site of infection, all using dosage forms well known to those of ordinary skill in the pharmaceutical arts.

The compounds used in the method of the present disclosure can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. The compounds can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Techniques and compositions for making dosage forms useful in the present disclosure are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington’s Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds used in the method of the present disclosure may also be administered in the form of liposome delivery systems, such as small unilamellar vesicles, large unilamallar vesicles, and multilamellar vesicles. Liposomes can be formed from a variety of phospholipids, such as cholesterol, stearylamine, or phosphatidylcholines. The compounds may be administered as components of tissue-targeted emulsions.

The compounds used in the method of the present disclosure may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington’s Pharmaceutical Sciences, Mack Publishing Company, a standard reference text in this field.

The compounds used in the method of the present disclosure may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials to make them compatible with the type of injection or delivery system chosen.

In specific aspects, a disclosed compound may be administered at a dosage unit of about 0.1 mg to about 1000 mg, or about 1 mg to about 400 mg, or about 5 mg to about 300 mg, about 10 mg to about 200 mg, about 100 mg to about 200 mg, or at least 400 mg, at least 300 mg, at least 200 mg, at least 150 mg, at least 120 mg, at least 100 mg, at least 50 mg, at least 40 mg, at least 30 mg, at least 20 mg, at least 10 mg, at least 9 mg, at least 8.5 mg, at least 8 mg, at least 7.5 mg, at least 7 mg, at least 6.5 mg, at least 6 mg, at least 5.5 mg, at least 5 mg, at least 4.5 mg, at least 4 mg, at least 3.5 mg, at least 3 mg, at least 2.5 mg, at least 2 mg, or at least 1 mg.

In specific exemplifications, for a compound of the structure:

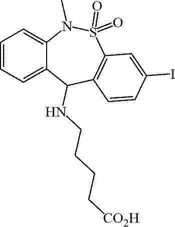 [SMILES: CN1c2ccccc2C(NCCCCC(=O)O)c2ccc(I)cc2S1(=O)=O]

or its pharmaceutically acceptable salts or esters thereof, administration may be carried out at a dosage unit of about 1 mg to about 40 mg, or about 2 mg to about 20 mg, or about 4 mg to about 12 mg, or at least 20 mg, at least 18 mg, at least 16 mg, at least 14 mg, at least 12 mg, at least 10 mg, at least 9.5 mg, at least 9 mg, at least 8.5 mg, at least 8 mg, at least 7.5 mg, at least 7 mg, at least 6.5 mg, at least 6 mg, at least 5.5 mg, at least 5 mg, at least 4.5 mg, at least 4 mg, at least 3.5 mg, at least 3 mg, at least 2.5 mg, at least 2 mg, or at least 1 mg.

The dosage units as noted herein may be administered once per day, twice per day, three times per day, four times per day, or more as needed. Administration of a dosage unit once or twice per day is specifically noted. The dosage and administration regime may be adjusted for pediatric, geriatric, overweight, underweight, or other patients, where required. The dosage and administration regime may also be adapted for extended release formulations. All such modifications can be made in accordance with known methods.

Each embodiment disclosed herein is contemplated as being applicable to each of the other disclosed embodiments. Thus, all combinations of the various elements described herein are within the scope of this disclosure.

This invention will be better understood by reference to the Experimental Details which follow, but those skilled in the art will readily appreciate that the specific experiments detailed are only illustrative and that the invention is described in the claims which follow thereafter.

### EXPERIMENTAL DETAILS

### Example 1. Preparation of Compounds

General Considerations. Reagents and solvents were obtained from commercial sources and were used without further purification unless otherwise stated. All compounds were prepared in racemic form. All reactions were performed in flame-dried glassware under an argon atmosphere unless otherwise stated, and monitored by TLC using solvent mixtures appropriate to each reaction. All column chromatography was performed on silica gel (40-63 µm). Preparative TLC was conducted on 20×20 cm plates coated with a 1 mm silica layer. Nuclear magnetic resonance spectra were recorded on Bruker 400 or 500 MHz instruments as indicated. Chemical shifts are reported as δ values in ppm referenced to CDCl₃ (¹H NMR = 7.26 and ¹³C NMR = 77.16) or CD₃OD (¹H NMR = 3.31 and ¹³C NMR = 49.00). Multiplicity is indicated as follows: s (singlet); d (doublet); t (triplet); q (quartet); p (pentet); dd (doublet of doublets); ddd (doublet of doublet of doublets); dt (doublet of triplets); td (triplet of doublets); m (multiplet); br (broad). All carbon peaks are rounded to one decimal place unless such rounding would cause two close peaks to become identical; in these cases, two decimal places are retained. Low-resolution mass spectra were recorded on a JEOL LCmate (ionization mode: APCI+). For compounds 4 and 5 mass spectra are reported for carbocations corresponding to loss of OH or Cl respectively.

Those having ordinary skill in the art of organic synthesis will appreciate that modifications to general procedures and synthetic routes contained in this application can be used to yield additional derivatives and structurally diverse compounds. Suitable organic transformations are described in March’s Advanced Organic Chemistry: Reactions, Mechanisms, and Structure (Wiley-Interscience; 6^{th} edition, 2007), the content of which is hereby incorporated by reference.

### Preparation of Diarylthiazepinones (3)

Scheme 1. Preparation of diarylthiazepinones.

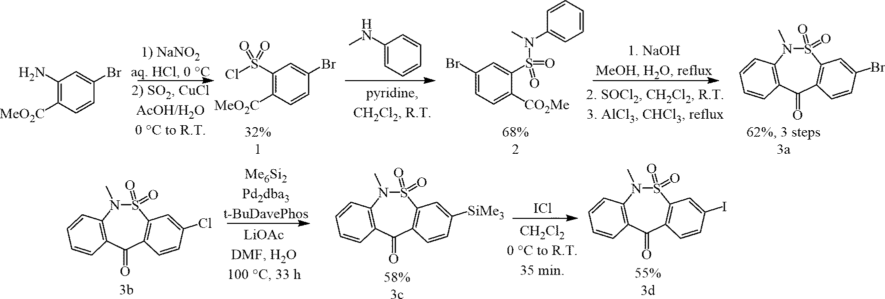

Methyl 4-bromo-2-(chlorosulfonyl)benzoate (1). A suspension of methyl 2-amino-4-bromobenzoate (10.35 g, 45.0 mmol) in 20% aqueous HCl (29 mL) was sonicated for several minutes and warmed slightly until all clumps were broken up and the mixture was a uniform suspension of fine particles. This mixture was cooled to 0° C., and a solution of NaNO₂ (3.11 g, 45.0 mmol) in water (7.5 mL) was added dropwise, maintaining the internal temperature below 5° C. The resulting mixture was then stirred for 2 h at 0° C. Simultaneously, a solution of SO₂ (23.1 g, 360 mmol) in AcOH (36.0 mL) and water (3.75 mL) was prepared by bubbling the gas though the mixed solvents at 0° C. until the mass had increased by the required amount. To this SO₂ solution was then added CuCl (1.11 g, 11.25 mmol) followed by the diazonium salt solution portionwise over 30 minutes at 0° C. The resulting mixture was then stirred for 1 h at 0° C. and 1 h at room temperature, poured into ice water (150 mL), and extracted with CH₂Cl₂ (3 × 50 mL). The combined organics were poured into saturated aqueous NaHCO₃ (75 mL), and solid NaHCO₃ was added carefully until effervescence ceased. The organic phase was then separated, washed with brine (50 mL), dried over Na₂SO₄, and concentrated to provide the crude sulfonyl chloride as a waxy brown solid (6.11 g, 74 mass% product by NMR, 32% yield). This material was used in the next step without further purification.

Methyl 4-bromo-2- (N-methyl-N-phenylsulfamoyl) benzoate (2). To a solution of crude methyl 4-bromo-2-(chlorosulfonyl)benzoate 1 (6.04 g, 74% pure, 14.25 mmol) in anhydrous pyridine (10.7 mL) was added N-methylaniline (1.71 mL, 1.68 g, 15.68 mmol) at room temperature, and the resulting mixture was stirred for 1 h. The reaction mixture was then diluted with CH₂Cl₂ (100 mL) and washed with 7% aqueous HCl (2 × 100 mL), brine (100 mL), saturated aqueous NaHCO₃ (100 mL), and brine again (100 mL), dried over Na₂SO₄, and concentrated to give a yellow-brown oil (2.41 g). This material was purified by column chromatography (9:1 hexanes:EtOAc, 4 column volumes → 8:2 hexanes:EtOAc, 4 column volumes) to provide pure sulfonamide 2 as an oil (3.71 g, 68%). ¹H NMR (500 MHz, CDCl₃) δ 7.69 (dd, J = 8.2, 1.9 Hz, 1H), 7.52 (d, J = 1.9 Hz, 1H), 7.38 - 7.29 (m, 4H), 7.21 - 7.17 (m, 2H), 3.82 (s, 3H), 3.30 (s, 3H); ¹³C NMR (126 MHz, CDCl₃) δ 167.7, 141.0, 137.1, 135.5, 132.8, 132.3, 129.8, 129.3, 128.0, 127.4, 123.9, 53.4, 39.0.

3-Bromomethyldibenzo[c,f][1,2]thiazepin-11(6H)-one 5,5-dioxide (3a). To a solution of sulfonamide 2 (3.69 g, 9.60 mmol) in MeOH (24 mL) was added water (12 mL) and NaOH (1.15 g, 28.80 mmol) and the mixture was refluxed for 1 h. Most of the MeOH was then removed in vacuo and the resulting clumpy white mixture was diluted with water (30 mL), acidified with 10% aqueous HCl (20 mL), and extracted with CH₂Cl₂ (50 mL, 2 × 20 mL). The combined organics were dried over Na₂SO₄ and concentrated to provide the carboxylic acid as a pale-pink glass (3.46 g), which was used in the next step without further purification. The carboxylic acid (3.43 g, 9.26 mmol) was dissolved in thionyl chloride (15 mL), and the solution was stirred for 13 h at room temperature. The volatiles were then removed to provide the crude acyl chloride as a yellow-orange oil. This material was dissolved in CHCl₃ (40 mL), aluminum chloride (3.95 g, 29.63 mmol) was added, and the mixture was refluxed for 1 h. The reaction was then cooled to room temperature, quenched with ice water (150 mL), and extracted with CH₂Cl₂ (3 × 50 mL). The combined organics were filtered through a silica plug, washing with additional CH₂Cl₂ until all of the product had passed through, and the filtrate was concentrated to provide an off-white solid. This material was recrystallized from MeOH (~250 mL) to provide the pure ketone 3a as cream colored needles (2.08 g, 62% over 3 steps). ¹H NMR (500 MHz, CDCl₃) δ 8.30 (dd, J - 8.1, 1.5 Hz, 1H), 8.11 (t, J = 1.0 Hz, 1H), 7.84 (d, J = 1.1 Hz, 2H), 7.68 - 7.62 (m, 1H), 7.41 - 7.37 (m, 1H), 7.35 (dd, J = 8.1, 0.7 Hz, 1H), 3.36 (s, 3H); ¹³C NMR (126 MHz, CDCl₃) δ 189.8, 141.5, 138.5, 136.4, 135.1, 135.0, 133.4, 132.3, 131.0, 128.4, 126.9, 126.4, 124.8, 39.2; LR-MS calcd. for C₁₄H₁₁BrNO₃S [M+H]⁺ 351.96, found 351.85.

3-Chloro-6-methyldibenzo[c,f][1,2]thiazepin-11(6H)-one 5,5-dioxide (3b). Ketone 3b was purchased from Ark Pharm Inc. (Libertyville, IL) and used without further purification.

6-Methyl-3-(trimethylsilyl)dibenzo[c,f][1,2]thiazepin-11(6H)-one 5,5-dioxide (3c). Ketone 3c was prepared from the aryl chloride utilizing the trimethylsilylation procedure of Buchwald (McNeill, E. et al. 2007). Ketone 3b (462 mg, 1.50 mmol), Pd₂dba₃ (20.6 mg, 0.0225 mmol), t-BuDavePhos (2′-(Di-tert-butylphosphino)-N,N-dimethyl-biphenyl-2-amine, 46.1 mg, 0.135 mmol), and LiOAc (495 mg, 7.50 mmol) were combined under argon. Anhydrous DMF (4.5 mL), water (54 µL, 3.00 mmol), and hexamethyldisilane (369 µL, 1.80 mmol) were then added, and the resulting orange-brown mixture was heated to 100° C. for 33 h. After cooling to room temperature, the reaction mixture was diluted with water (20 mL) and extracted with Et₂O (3 × 10 mL). The combined organics were washed with water (10 mL), dried over Na₂SO₄, and concentrated to yield a yellow crystalline solid. This crude material was recrystallized from MeOH to obtain pure ketone 3c as fine yellow needles (301 mg, 58%). ¹H NMR (500 MHz, CDCl₃) δ 8.30 (dd, J = 8.1, 1.6 Hz, 1H), 8.04 (d, J = 0.8 Hz, 1H), 7.91 (d, J = 7.5 Hz, 1H), 7.85 (dd, J = 7.6, 1.1 Hz, 1H), 7.63 (ddd, J = 8.1, 7.3, 1.7 Hz, 1H), 7.41 - 7.29 (m, 2H), 3.35 (s, 3H), 0.36 (s, 9H); ¹³C NMR (126 MHz, CDCl₃) δ 191.2, 147.4, 141.9, 138.3, 136.6, 136.0, 134.8, 132.1, 131.2, 130.5, 129.7, 126.0, 124.6, 39.1, -1.2; LR-MS calcd. for C₁₇H₂₀NO₃SSi [M+H]⁺ 346.09, found 345.86.

3-Iodomethyldibenzo[c,f][1,2]thiazepin-11(6H)-one 5,5-dioxide (3d). To a solution of trimethylsilylketone 3c (108 mg, 0.313 mmol) in anhydrous CH₂Cl₂ (0.94 mL) at 0° C. was added a solution of iodine monochloride (173 mg, 1.06 mmol) in anhydrous CH₂Cl₂ (0.63 mL) dropwise over 3 min. The resulting dark-brown solution was allowed to warm to room temperature, stirred for 35 min (extended reaction times produce polyiodinated byproducts), and quenched with saturated aqueous Na₂S₂O₃ (3 mL). The resulting mixture was diluted with water (15 mL) and extracted with CH₂Cl₂ (2 × 15 mL). The combined organics were washed with water (15 mL), dried over Na₂SO₄, and concentrated to yield a yellow solid. This material was purified by column chromatography (1:1 CH₂Cl₂:hexanes) to yield impure product. This crude product was recrystallized from MeOH and the resulting fine-white needles were dissolved in CH₂Cl₂ and concentrated, causing a second crystallization to occur once most of the solvent had been removed. The powdery white crystals thus obtained were washed with ice-cold MeOH and dried to yield the pure ketone 3d (68.4 mg, 55%). ¹H NMR (400 MHz, CDCl₃) δ 8.32 - 8.27 (m, 2H), 8.06 (dd, J = 8.1, 1.7 Hz, 1H), 7.69 - 7.62 (m, 2H), 7.38 (ddd, J - 8.2, 7.3, 1.1 Hz, 1H), 7.34 (dd, J = 8.1, 0.9 Hz, 1H), 3.35 (s, 3H); ¹³C NMR (101 MHz, CDCl₃) δ 190.1, 142.4, 141.5, 138.1, 135.6, 135.1, 133.9, 133.1, 132.2, 131.0, 126.3, 124.7, 98.7, 39.2; LR-MS calcd. for C₁₄H₁₁INO₃S [M+H]⁺ 399.95, found 399.78.

### Preparation of Diarylthiazepinyl Chlorides (5)

Scheme 2. Preparation of diarylthiazepinyl chlorides.

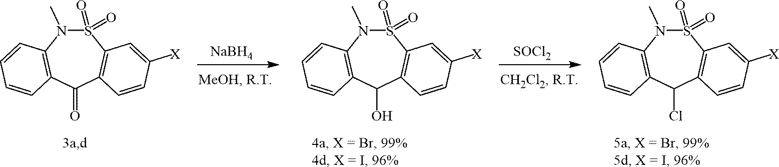

General Procedure for Preparation of Diarylthiazepinyl Alcohols (4). Sodium borohydride (2 equivalents) was added to an ice-cooled solution (or suspension) of the appropriate ketone 3 (1 equivalent) in MeOH (0.143 M based on 3) and the mixture was allowed to warm to room temperature and stirred until TLC indicated the complete consumption of starting material. The reaction was then quenched with saturated aqueous ammonium chloride (5 mL per mmol 3) and saturated aqueous NaHCO₃ (5 mL per mmol 3). The MeOH was evaporated and the precipitate was filtered, washed with water, and dried (alternatively, the residue was extracted with EtOAc and the combined organic layers were washed with water, dried over Na₂SO₄, filtered, and concentrated). The resulting product 4 was used in the next step without further purification.

3-Bromo-11-hydroxy-6-methyl-6,11-dihydrodibenzo [c,f] [1,2]thiazepine 5,5-dioxide (4a). The product 4a was prepared according to the general procedure and obtained as a white solid (1.85 g, 99%). ¹H NMR (500 MHz, CDCl₃) δ 8.11 (d, J = 2.0 Hz, 1H), 7.73 (dd, J = 8.2, 2.0 Hz, 1H), 7.61 (dd, J = 7.7, 1.0 Hz, 1H), 7.57 (d, J = 8.2 Hz, 1H), 7.41 (td, J = 7.6, 1.5 Hz, 1H), 7.37 - 7.30 (m, 2H), 5.93 (d, J = 9.7 Hz, 1H), 4.16 (d, J = 9.7 Hz, 1H), 3.20 (s, 3H); ¹³C NMR (126 MHz, CDCl₃) δ 138.9, 138.8, 136.9, 136.4, 135.3, 131.8, 131.5, 131.0, 130.1, 127.9, 127.0, 122.7, 76.2, 39.4; LR-MS calcd. for C₁₄H₁₁BrNO₂S [M-OH]⁺ 335.97, found 335.89.

11-Hydroxy-3-iodo-6-methyl-6,11-dihydrodibenzo[c,f][1,2]thiazepine 5,5-dioxide (4d). The product 4d was prepared according to the general procedure and obtained as a white solid (74.8 mg, 95%). ¹H NMR (500 MHz, CDCl₃) (observed as a ~4:1 ratio of 2 conformers, resulting in partial integrals) δ 8.27 (d, J = 1.8 Hz, 1H), 7.99 (dd, J = 7.7, 1.1 Hz, 0.2H), 7.93 (dd, J = 8.1, 1.8 Hz, 0.8H), 7.68 (d, J = 7.2 Hz, 0.2H), 7.65 - 7.58 (m, 1H), 7.53 (td, J = 7.6, 1.3 Hz, 0.2H), 7.44 -7.37 (m, 1.8H), 7.37 - 7.29 (m, 1.8H), 5.92 (s, 1H), 4.40 (d, J = 9.6 Hz, 0.2H), 4.15 (d, J = 7.5 Hz, 0.8H), 3.20 (s, 2.4H), 3.14 (s, 0.6H); ¹³C NMR (126 MHz, CDCl₃) (additional peaks due to conformers) δ 142.4, 138.8, 138.7, 137.5, 136.5, 135.4, 133.6, 132.1, 131.7, 131.4, 130.6, 130.1, 130.0, 128.9, 128.4, 127.9, 127.6, 127.0, 126.9, 93.6, 76.2, 39.3; LR-MS calcd. for C₁₄H₁₁INO₂S [M-OH]⁺ 383.96, found 383.71.

General Procedure for Preparation of Diarylthiazepinyl Chlorides (5). Thionyl chloride (6 equivalents) was added dropwise to a solution of the appropriate alcohol 4 (1 equivalent) in anhydrous CH₂Cl₂ (0.143 M based on 4). The reaction mixture was stirred overnight at room temperature and then concentrated to provide the corresponding chloride 5, which was used directly in the following reactions without further purification.

3-Bromo-11-chloro-6-methyl-6,11-dihydrodibenzo[c,f][1,2]thiazepine 5,5-dioxide (5a). The product 5a was prepared according to the general procedure and obtained as a white solid (1.92 g, 99%). ¹H NMR (400 MHz, CDCl₃) δ 8.14 (d, J = 2.1 Hz, 1H), 7.66 (dd, J = 8.3, 2.1 Hz, 1H), 7.55 - 7.49 (m, 2H), 7.45 - 7.40 (m, 2H), 7.39 - 7.33 (m, 1H), 6.10 (s, 1H), 3.58 (s, 3H); ¹³C NMR (101 MHz, CDCl₃) δ 142.0, 139.2, 137.6, 135.6, 134.1, 133.0, 131.7, 131.0, 130.2, 129.5, 129.0, 124.3, 63.7, 39.3; LR-MS cald. for C₁₄H₁₁BrNO₂S [M-Cl]⁺ 335.97, found 335.79.

3,11-dichloro-6-methyl-6,11-dihydrodibenzo[c,f][1,2]thiazepine 5,5-dioxide (5b). Chloride 5b was purchased from Ark Pharm Inc. (Libertyville, IL) and used without further purification.

11-Chloro-3-iodo-6-methyl-6,11-dihydrodibenzo[c,f][1,2]thiazepine 5,5-dioxide (5d). The product 5d was prepared according to the general procedure and obtained as a gray solid (73.4 mg, 96%). ¹H NMR (400 MHz, CDCl₃) δ 8.32 (d, J = 1.8 Hz, 1H), 7.87 (dd, J = 8.2, 1.8 Hz, 1H), 7.57 - 7.48 (m, 2H), 7.43 (d, J = 7.1 Hz, 1H), 7.39 - 7.33 (m, 1H), 7.27 (d, J = 7.1 Hz, 1H), 6.08 (s, 1H), 3.57 (s, 3H); ¹³C NMR (101 MHz, CDCl₃) δ 141.8, 141.5, 139.3, 137.6, 136.7, 134.7, 132.9, 131.7, 130.1, 129.5, 129.0, 95.4, 63.8, 39.3; LR-MS calcd. for C₁₄H₁₁INO₂S [M-Cl] ⁺ 383.96, found 383.70.

### Preparation of Diarylthiazepinamine Esters (6)

Scheme 3. Preparation of diarylthiazepinamine esters.

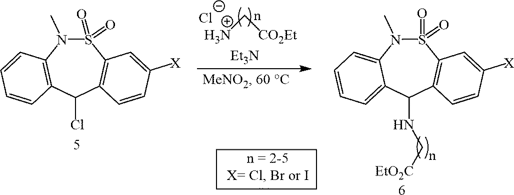

General Procedure for Preparation of Diarylthiazepinamines. To a suspension of the appropriate chloride 5 (1 equivalent) in nitromethane (0.5 M based on 5) was added an aminoester hydrochloride (1.2 equivalents) and Et₃N (2.4 equivalents) and the mixture was warmed to 60° C. and left to stir until TLC indicated that the reaction was complete (typically <1 h). The reaction mixture was then concentrated in vacuo and purified directly by column chromatography. Alternatively, the concentrated reaction residue was partitioned between Et₂O (20 mL per mmol 5) and water (20 mL per mmol 5). The ethereal layer was separated and the aqueous extracted again with Et₂O (20 mL per mmol 5). The combined organics were washed with water (20 mL per mmol 5) and 10% NH₄OH (20 mL per mmol 5), dried over Na₂SO₄, and concentrated to yield the product. If necessary, the product was further purified by column chromatography or preparative TLC.

### Ethyl 3-((3-bromo-6-methyl-5,5-dioxido-6,11-dihydrodibenzo[c,f][1,2] thiazepin-11-yl)amino)propanoate (6a).

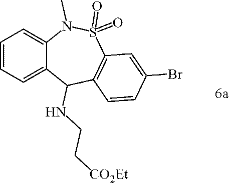 [SMILES: CCOC(=O)CCNC1c2ccccc2N(C)S(=O)(=O)c2cc(Br)ccc21]

The product 6a was prepared according to the general procedure and purified by preparative TLC (20:1 CH₂Cl₂: Et₂O) to provide a viscous, pale-yellow oil (30.8 mg, 68%). ¹H NMR (500 MHz, CDCl₃) δ 8.09 (d, J = 2.0 Hz, 1H), 7.61 (dd, J = 8.3, 2.0 Hz, 1H), 7.42 - 7.33 (m, 4H), 7.29 (td, J = 7.5, 1.3 Hz, 1H), 5.03 (s, 1H), 4.12 (q, J = 7.1 Hz, 2H), 3.39 (s, 3H), 2.80 - 2.69 (m, 2H), 2.56 - 2.43 (m, 2H), 2.47 (br s, 1H), 1.24 (t, J = 7.1 Hz, 3H); ¹³C NMR (126 MHz, CDCl₃) δ 172.7, 140.7, 138.8, 138.6, 137.3, 135.4, 131.3, 131.2, 129.9, 129.6, 128.3, 128.0, 122.1, 65.9, 60.7, 43.6, 38.6, 34.8, 14.3.

### Ethyl 3-((3-iodo-6-methyl-5,5-dioxido-6,11-dihydrodibenzo[c,f] [1,2] thiazepin-11-yl)amino)propanoate (6b).

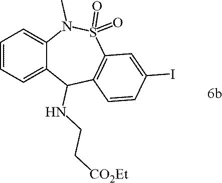 [SMILES: CCOC(=O)CCNC1c2ccccc2N(C)S(=O)(=O)c2cc(I)ccc21]

The product 6b was prepared according to the general procedure and purified by preparative TLC (20:1 CH₂Cl₂:Et₂O) to provide a viscous, colorless oil (46.8 mg, 94%). ¹H NMR (500 MHz, CDCl₃) δ 8.26 (d, J = 1.8 Hz, 1H), 7.81 (dd, J - 8.1, 1.8 Hz, 1H), 7.41 - 7.37 (m, 2H), 7.35 (td, J = 7.4, 1.6 Hz, 1H), 7.28 (td, J = 7.5, 1.4 Hz, 1H), 7.25 (d, J = 8.2 Hz, 1H), 5.02 (s, 1H), 4.12 (q, J = 7.1 Hz, 2H), 3.38 (s, 3H), 2.80 - 2.69 (m, 2H), 2.57 - 2.43 (m, 2H), 2.44 (br s, 1H), 1.24 (t, J = 7.1 Hz, 3H); ¹³C NMR (126 MHz, CDCl₃) δ 172.7, 141.3, 140.6, 138.7, 138.6, 137.9, 136.8, 131.2, 129.8, 129.5, 128.3, 128.0, 93.0, 65.9, 60.7, 43.5, 38.5, 34.8, 14.3.

### Ethyl 4-((3-bromo-6-methyl-5,5-dioxido-6,11-dihydrodibenzo[c,f] [1,2]thiazepin-11-yl)amino)butanoate (6c).

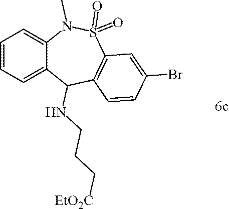 [SMILES: CCOC(=O)CCCNC1c2ccccc2N(C)S(=O)(=O)c2cc(Br)ccc21]

The product 6c was prepared according to the general procedure and purified directly by column chromatography (20:1 CH₂Cl₂:Et₂O, 4 column volumes → 7:3 CH₂Cl₂:Et₂O, 2 column volumes) to provide a viscous, nearly colorless oil (43.5 mg, 93%). ¹H NMR (500 MHz, CDCl₃) δ 8.10 (d, J = 2.0 Hz, 1H), 7.61 (dd, J = 8.2, 2.1 Hz, 1H), 7.40 - 7.33 (m, 4H), 7.31 - 7.27 (m, 1H), 5.00 (s, 1H), 4.09 (q, J = 7.1 Hz, 2H), 3.35 (s, 3H), 2.51 (t, J = 6.9 Hz, 2H), 2.41 - 2.29 (m, 2H), 2.13 (br s, 1H), 1.81 (p, J = 7.1 Hz, 2H), 1.22 (t, J = 7.1 Hz, 3H); ¹³C NMR (126 MHz, CDCl₃) δ 173.6, 140.5, 138.7, 138.4, 137.4, 135.3, 131.43, 131.37, 130.2, 129.5, 128.2, 128.0, 122.0, 66.0, 60.5, 47.3, 38.8, 32.2, 25.3, 14.3.

### Ethyl 5-((3-bromo-6-methyl-5,5-dioxido-6,11-dihydrodibenzo[c,f] [1,2]thiazepin-11-yl)amino)pentanoate (6d).

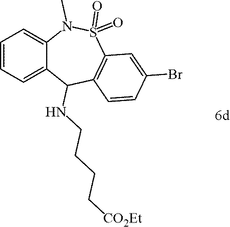 [SMILES: CCOC(=O)CCCCNC1c2ccccc2N(C)S(=O)(=O)c2cc(Br)ccc21]

The product 6d was prepared according to the general procedure and purified by preparative TLC (6:4 hexanes:EtOAc) to provide a viscous, colorless oil (40.8 mg, 85%). ¹H NMR (500 MHz, CDCl₃) δ 8.10 (d, J = 2.1 Hz, 1H), 7.62 (dd, J = 8.2, 2.1 Hz, 1H), 7.40 - 7.34 (m, 4H), 7.32 - 7.27 (m, 1H), 4.98 (s, 1H), 4.10 (q, J = 7.1 Hz, 2H), 3.36 (s, 3H), 2.47 (t, J = 7.0 Hz, 2H), 2.27 (t, J = 7.4 Hz, 2H), 2.07 (br s, 1H), 1.69 - 1.59 (m, 2H), 1.57 - 1.46 (m, 2H), 1.24 (t, J = 7.1 Hz, 3H); ¹³C NMR (126 MHz, CDCl₃) δ 173.6, 140.5, 138.7, 138.6, 137.4, 135.3, 131.5, 131.4, 130.2, 129.5, 128.3, 128.1, 122.1, 66.3, 60.4, 47.8, 38.8, 34.2, 29.6, 22.7, 14.4.

### Ethyl 5-((3-iodo-6-methyl-5,5-dioxido-6,11-dihydrodibenzo[c,f][1,2] thiazepin-11-yl)amino)pentanoate (6e).

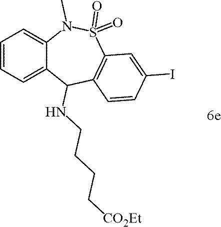 [SMILES: CCOC(=O)CCCCNC1c2ccccc2N(C)S(=O)(=O)c2cc(I)ccc21]

The product 6e was prepared according to the general procedure and purified by preparative TLC (20:1 CH₂Cl₂:Et₂O) to provide a viscous, nearly colorless oil (30.1 mg, 57%). ¹H NMR (500 MHz, CDCl₃) δ 8.27 (d, J = 1.8 Hz, 1H), 7.81 (dd, J = 8.1, 1.8 Hz, 1H), 7.40 - 7.33 (m, 3H), 7.31 - 7.26 (m, 1H), 7.21 (d, J = 8.2 Hz, 1H), 4.97 (s, 1H), 4.11 (q, J = 7.1 Hz, 2H), 3.36 (s, 3H), 2.48 (t, J = 7.0 Hz, 2H), 2.27 (t, J = 7.4 Hz, 2H), 2.06 (br s, 1H), 1.69 - 1.59 (m, 2H), 1.57 - 1.46 (m, 2H), 1.24 (t, J = 7.1 Hz, 3H); ¹³C NMR (126 MHz, CDCl₃) δ 173.6, 141.3, 140.6, 138.7, 138.1, 137.0, 131.5, 130.2, 129.5, 128.2, 128.1, 92.9, 66.3, 60.4, 47.8, 38.8, 34.2, 29.6, 22.8, 14.4.

### Ethyl 6-((3-bromo-6-methyl-5,5-dioxido-6,11-dihydrodibenzo[c,f][1,2] thiazepin-11-yl)amino)hexanoate (6f).

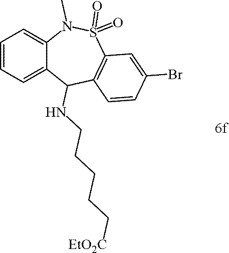 [SMILES: CCOC(=O)CCCCCNC1c2ccccc2N(C)S(=O)(=O)c2cc(Br)ccc21]

The product 6f was prepared according to the general procedure and purified directly by column chromatography (20:1 CH₂Cl₂:Et₂O, 4 column volumes → 7:3 CH₂Cl₂:Et₂O, 2 column volumes) to provide a viscous, pale-yellow oil (43.2 mg, 87%). ¹H NMR (400 MHz, CDCl₃) δ 8.10 (d, J = 2.0 Hz, 1H), 7.62 (dd, J = 8.2, 2.0 Hz, 1H), 7.43 - 7.33 (m, 4H), 7.32 - 7.27 (m, 1H), 5.01 (s, 1H), 4.10 (q, J = 7.1 Hz, 2H), 3.35 (s, 3H), 2.54 - 2.40 (m, 2H), 2.26 (t, J = 7.4 Hz, 2H), 2.21 (br s, 1H), 1.64 - 1.55 (m, 2H), 1.55 - 1.45 (m, 2H), 1.37 - 1.28 (m, 2H), 1.23 (t, J = 7.1 Hz, 3H); ¹³C NMR (101 MHz, CDCl₃) δ 173.7, 140.6, 138.8, 138.3, 137.2, 135.4, 131.6, 131.3, 130.3, 129.6, 128.2, 128.1, 122.1, 66.3, 60.4, 48.0, 38.8, 34.3, 29.8, 26.9, 24.9, 14.4.

### Ethyl 6-((3-iodo-6-methyl-5,5-dioxido-6,11-dihydrodibenzo[c,f][1,2] thiazepin-11-yl)amino)hexanoate (6 g).

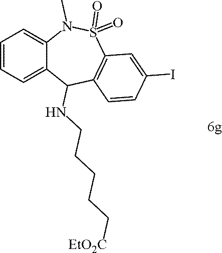 [SMILES: CCOC(=O)CCCCCNC1c2ccccc2N(C)S(=O)(=O)c2cc(I)ccc21]

The product 6g was prepared according to the general procedure and purified directly by column chromatography (20:1 CH₂Cl₂:Et₂O, 4 column volumes → 7:3 CH₂Cl₂:Et₂O, 2 column volumes) to provide a viscous, pale-yellow oil (47.4 mg, 87%). ¹H NMR (400 MHz, CDCl₃) δ 8.26 (d, J = 1.8 Hz, 1H), 7.81 (dd, J = 8.1, 1.8 Hz, 1H), 7.40 - 7.27 (m, 4H), 7.21 (d, J = 8.2 Hz, 1H), 4.96 (s, 1H), 4.10 (q, J = 7.1 Hz, 2H), 2.46 (t, J = 7.1 Hz, 2H), 2.26 (t, J = 7.4 Hz, 2H), 1.99 (br s, 1H), 1.65 - 1.54 (m, 2H), 1.54 - 1.44 (m, 2H), 1.38 - 1.27 (m, 2H), 1.23 (t, J = 7.1 Hz, 3H); ¹³C NMR (101 MHz, CDCl₃) δ 173.7, 141.2, 140.6, 138.8, 138.7, 138.2, 136.9, 131.4, 130.1, 129.4, 128.2, 128.1, 92.9, 66.3, 60.3, 48.0, 38.7, 34.3, 29.9, 26.9, 24.9, 14.4.

### Ethyl 3-((3-chloro-6-methyl-5,5-dioxido-6,11-dihydrodibenzo[c,f] [1,2]thiazepin-11-yl)amino)propanoate (6h).

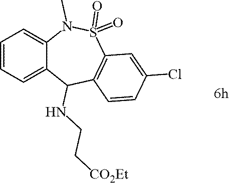 [SMILES: CCOC(=O)CCNC1c2ccccc2N(C)S(=O)(=O)c2cc(Cl)ccc21]

The product 6h was prepared according to the general procedure and purified directly by column chromatography (20:1 CH₂Cl₂:Et₂O, 3 column volumes → 7:3 CH₂Cl₂:Et₂O, 3 column volumes) to provide a viscous, colorless oil (393 mg, 96%). ¹H NMR (400 MHz, CDCl₃) δ 7.96 - 7.93 (m, 1H), 7.49 - 7.43 (m, 2H), 7.42 - 7.33 (m, 3H), 7.31 - 7.26 (m, 1H), 5.05 (s, 1H), 4.12 (q, J = 7.1 Hz, 2H), 3.39 (s, 3H), 2.83 - 2.68 (m, 2H), 2.59 - 2.41 (m, 2H), 2.51 (br s, 1H), 1.24 (t, J = 7.1 Hz, 3H) ; ¹³C NMR (101 MHz, CDCl₃) δ 172.7, 140.6, 138.8, 138.6, 136.9, 134.5, 132.4, 131.1, 129.9, 129.5, 128.5, 128.3, 128.1, 65.9, 60.7, 43.6, 38.6, 34.9, 14.3.

### Ethyl 5-((3-chloro-6-methyl-5,5-dioxido-6,11-dihydrodibenzo[c,f] [1,2]thiasepin-11-yl)amino)pentanoate (6i).

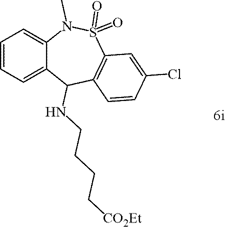 [SMILES: CCOC(=O)CCCCNC1c2ccccc2N(C)S(=O)(=O)c2cc(Cl)ccc21]

The product 6i was prepared according to the general procedure and purified by column chromatography (40:1 CH₂Cl₂:Et₂O, 4 column volumes → 20:1 CH₂Cl₂:Et₂O, 2 column volumes → 7:3 CH₂Cl₂:Et₂O, 2 column volumes) to provide a viscous, nearly colorless oil (256 mg, 59%). ¹H NMR (400 MHz, CDCl₃) δ 7.96 (d, J = 2.1 Hz, 1H), 7.48 - 7.33 (m, 5H), 7.32 -7.27 (m, 1H), 5.00 (s, 1H), 4.11 (q, J = 7.1 Hz, 2H), 3.36 (s, 3H), 2.48 (t, J = 17.0 Hz, 2H), 2.27 (t, J = 7.3 Hz, 2H), 2.08 (br s, 1H), 1.71 - 1.57 (m, 2H), 1.57 - 1.46 (m, 2H), 1.24 (t, J = 7.1 Hz, 3H); ¹³C NMR (101 MHz, CDCl₃) δ 173.6, 140.5, 138.7, 137.0, 134.4, 132.3, 131.3, 130.3, 129.5, 128.6, 128.2, 128.1, 66.3, 60.4, 47.8, 38.8, 34.2, 29.6, 22.8, 14.4.

### Ethyl 6-((3-chloro-6-methyl-5,5-dioxido-6,11-dihydrodibenzo[c,f] [1,2]thiazepin-11-yl)amino)hexanoate (6j).

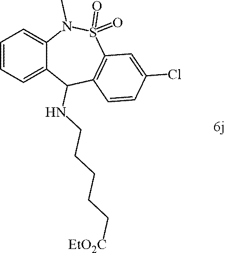 [SMILES: CCOC(=O)CCCCCNC1c2ccccc2N(C)S(=O)(=O)c2cc(Cl)ccc21]

The product 6j was prepared according to the general procedure and purified by preparative TLC (6:4 hexanes:EtOAc) to provide a viscous, pale-yellow oil (42.0 mg, 93%). ¹H NMR (500 MHz, CDCl₃) δ 7.95 (d, J = 2.1 Hz, 1H), 7.48 - 7.40 (m, 2H), 7.40 - 7.33 (m, 3H), 7.29 (td, J - 7.2, 1.8 Hz, 1H), 4.99 (s, 1H), 4.10 (q, J - 7.1 Hz, 2H), 3.36 (s, 3H), 2.46 (t, J - 7.1 Hz, 2H), 2.26 (t, J - 7.5 Hz, 2H), 1.96 (br s, 1H), 1.63 - 1.54 (m, 2H), 1.54 - 1.43 (m, 2H), 1.37 - 1.28 (m, 2H), 1.23 (t, J = 7.1 Hz, 3H); ¹³C NMR (126 MHz, CDCl₃) δ 173.8, 140.4, 138.7, 138.6, 136.9, 134.4, 132.3, 131.3, 130.2, 129.5, 128.6, 128.3, 128.1, 66.3, 60.4, 48.0, 38.8, 34.3, 29.9, 26.9, 24.9, 14.4.

### Preparation of Diarylthiazepinamine Carboxylic Acids (7)

Scheme 4. Preparation of diarylthiazepinamine carboxylic acids.

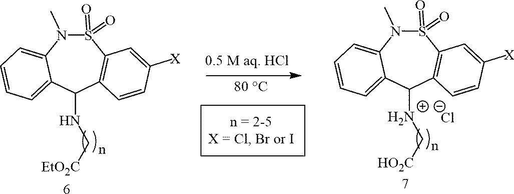

General Procedure for Preparation of Carboxylic Acids. The appropriate ester 6 (0.04 M concentration) was heated in aqueous HCl (0.5 M) at 80° C. until TLC indicated the complete consumption of starting material (typically <2 h). The reaction mixture was then concentrated and dried thoroughly in vacuo to provide the pure HCl salt of the corresponding amino carboxylic acid. If desired, the obtained products may be triturated with hexanes and then concentrated to provide a powdered solid.

### 3-(Hromo-6-methyl-5,5-dioxido-6,11-dihydrodibenzo[c,f] [1,2]thiazepin-11-yl)amino)propanoic acid hydrochloride salt (7a).

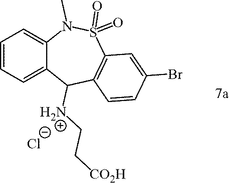 [SMILES: CN1c2ccccc2C([NH2+]CCC(=O)O)c2ccc(Br)cc2S1(=O)=O ; [Cl-]]

The product 7a was prepared according to the general procedure and obtained as a glassy, white foam (10.0 mg, 66%). ¹H NMR (500 MHz, CD₃OD) δ 8.22 (d, J = 2.0 Hz, 1H), 8.02 (dd, J - 8.1, 1.9 Hz, 1H), 7.86 (d, J = 8.1 Hz, 1H), 7.77 (d, J = 7.6 Hz, 1H), 7.65 (t, J = 7.5 Hz, 1H), 7.60 (d, J = 7.2 Hz, 1H), 7.49 (t, J = 7.1 Hz, 1H), 6.02 (s, 1H), 3.30 - 3.25 (m, 1H), 3.28 (s, 3H), 3.11 (dt, J = 12.5, 6.1 Hz, 1H), 2.82 - 2.69 (m, 2H); ¹³C NMR (126 MHz, CD₃OD) δ 174.2, 142.3, 142.1, 138.3, 137.2, 134.7, 133.6, 132.3, 129.3, 128.3, 128.2, 127.3, 126.7, 68.1, 44.4, 39.3, 30.7.

### 3-(Iodo-6-methyl-5,5-dioxido-6,11-dihydrodibanzo[c,f] [1,2]thiazepin-11-yl)amino)propanoic acid hydrochloride salt (7b).

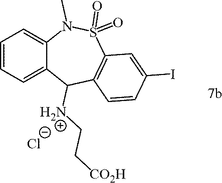 [SMILES: CN1c2ccccc2C([NH2+]CCC(=O)O)c2ccc(I)cc2S1(=O)=O ; [Cl-]]

The product 7b was prepared according to the general procedure and obtained as a glassy, white foam (10.0 mg, 99%). ¹H NMR (500 MHz, CD₃OD) δ 8.38 (d, J - 1.8 Hz, 1H), 8.22 (dd, J = 8.0, 1.8 Hz, 1H), 7.74 (dd, J - 7.8, 1.3 Hz, 1H), 7.68 - 7.62 (m, 2H), 7.59 (dd, J = 8.1, 1.2 Hz, 1H), 7.49 (td, J - 7.6, 1.3 Hz, 1H), 5.94 (s, 1H), 3.29 - 3.24 (m, 1H), 3.27 (s, 3H), 3.10 (ddd, J = 12.7, 7.2, 5.7 Hz, 1H), 2.80 - 2.68 (m, 2H); ¹³C NMR (126 MHz, CD₃OD) δ 174.2, 144.5, 142.4, 141.6, 138.0, 136.8, 134.6, 133.6, 129.2, 128.6, 128.4, 127.3, 98.1, 68.4, 44.3, 39.4, 30.7.

### 4-(Bromo-6-mothyl-5,5-dioxido-6,11-dihydrodibenzo[c,f][1,2] thiazepin-11-yl)amino)butanoic acid hydrochloride salt (7c).

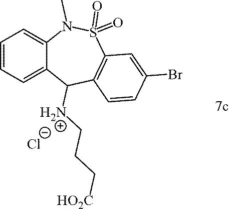 [SMILES: CN1c2ccccc2C([NH2+]CCCC(=O)O)c2ccc(Br)cc2S1(=O)=O ; [Cl-]]

The product 7c was prepared according to the general procedure and obtained as a white solid (15.3 mg, 88%). ¹H NMR (500 MHz, CD₃OD) δ 8.22 (d, J = 2.0 Hz, 1H), 8.01 (dd, J = 8.2, 2.1 Hz, 1H), 7.82 (d, J = 8.2 Hz, 1H), 7.72 (dd, J = 7.8, 1.0 Hz, 1H), 7.67 - 7.62 (m, 1H), 7.59 (dd, J = 8.0, 1.0 Hz, 1H), 7.51 - 7.46 (m, 1H), 5.94 (s, 1H), 3.26 (s, 3H), 3.07 (ddd, J = 12.4, 9.0, 5.9 Hz, 1H), 2.93 (ddd, J = 12.4, 8.9, 6.4 Hz, 1H), 2.42 (t, J = 6.8 Hz, 2H), 2.02 - 1.85 (m, 2H); ¹³C NMR (126 MHz, CD₃OD) δ 176.4, 142.4, 142.1, 138.2, 137.0, 134.6, 133.5, 132.2, 129.2, 128.53, 128.47, 127.6, 126.6, 67.7, 47.9, 39.5, 31.7, 22.2.

### 5-(Bromo-6-methyl-5,5-dioxido-6,11-dihydrodibenzo[c,f][1,2] thiazepin-11-yl)amino)pentanoic acid hydrochloride salt (7d).

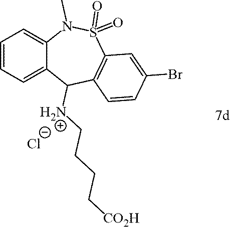 [SMILES: CN1c2ccccc2C([NH2+]CCCCC(=O)O)c2ccc(Br)cc2S1(=O)=O ; [Cl-]]

The product 7d was prepared according to the general procedure and obtained as a glassy, white foam (6.6 mg, 45%). ¹H NMR (400 MHz, CD₃OD) δ 8.23 (s, 1H), 8.03 (d, J = 8.1 Hz, 1H), 7.80 (d, J = 8.1 Hz, 1H), 7.71 (d, J = 7.6 Hz, 1H), 7.65 (t, J = 7.7 Hz, 1H), 7.58 (d, J = 8.0 Hz, 1H), 7.49 (t, J = 7.5 Hz, 1H), 5.92 (s, 1H), 3.23 (s, 3H), 3.04 -2.94 (m, 1H), 2.92 - 2.81 (m, 1H), 2.32 (t, J = 6.9 Hz, 2H), 1.79 -1.65 (m, 2H), 1.65 - 1.54 (m, 2H).

### 5-(Iodo-6-mothyl-5,5-dioxido-6,11-dihydrodibenzo[c,f] [1,2]thiazepin-11-y1)amino)pentanoic acid hydrochloride salt (7e).

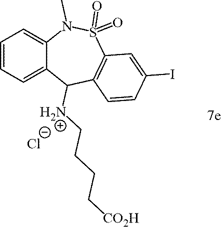 [SMILES: CN1c2ccccc2C([NH2+]CCCCC(=O)O)c2ccc(I)cc2S1(=O)=O ; [Cl-]]

The product 7e was prepared according to the general procedure and obtained as a white solid (10.5 mg, 65%). ¹H NMR (400 MHz, CD₃OD) δ 8.38 (d, J = 1.7 Hz, 1H), 8.22 (dd, J = 8.0, 1.7 Hz, 1H), 7.71 (d, J = 7.7 Hz, 1H), 7.68 - 7.60 (m, 2H), 7.57 (d, J = 7.1 Hz, 1H), 7.48 (t, J = 7.5 Hz, 1H), 5.88 (s, 1H), 3.23 (s, 3H), 3.04 - 2.93 (m, 1H), 2.90 - 2.80 (m, 1H), 2.31 (t, J = 6.9 Hz, 2H), 1.79 - 1.65 (m, 2H), 1.65 - 1.54 (m, 2H); ¹³C NMR (101 MHz, CD₃OD) δ 176.8, 144.5, 142.6, 141.4, 138.0, 136.7, 134.7, 133.5, 129.1, 128.8, 128.5, 127.1, 98.0, 68.0, 48.1, 39.7, 33.8, 26.4, 22.7.

### 6-(Bromo-6-methyl-5,5-dioxido-6,11-dihydrodibenzo[c,f] [1,2]thiazepin-11-yl)amino)hexanoic acid hydrochloride salt (7f).

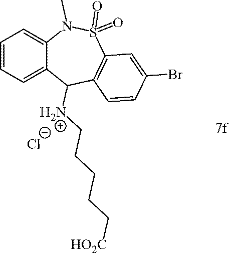 [SMILES: CN1c2ccccc2C([NH2+]CCCCCC(=O)O)c2ccc(Br)cc2S1(=O)=O ; [Cl-]]

The product 7f was prepared according to the general procedure and obtained as a white, glassy foam (18.2 mg, 94%). ¹H NMR (500 MHz, CD₃OD) δ 8.22 (d, J = 2.1 Hz, 1H), 8.03 (dd, J = 8.2, 2.1 Hz, 1H), 7.85 (d, J = 8.2 Hz, 1H), 7.76 (dd, J = 7.8, 1.2 Hz, 1H), 7.68 - 7.62 (m, 1H), 7.58 (dd, J = 8.1, 1.1 Hz, 1H), 7.49 (td, J = 7.7, 1.2 Hz, 1H), 5.99 (s, 1H), 3.24 (s, 3H), 2.98 (ddd, J = 11.9, 10.5, 5.5 Hz, 1H), 2.87 - 2.80 (m, 1H), 2.28 (t, J = 7.3 Hz, 2H), 1.77 - 1.62 (m, 2H), 1.62 - 1.54 (m, 2H), 1.39 - 1.30 (m, 2H); ¹³C NMR (126 MHz, CD₃OD) δ 177.2, 142.5, 141.9, 138.3, 137.2, 134.8, 133.5, 132.2, 129.2, 128.5, 128.4, 127.1, 126.6, 67.6, 48.2, 39.7, 34.3, 26.9, 26.6, 25.3.

### 6-(Iodo-6-methyl-5,5-dioxido-6,11-dihydrodibenzo[c,f] [1,2]thiazepin-11-yl)amino)hexanoic acid hydrochloride salt (7g).

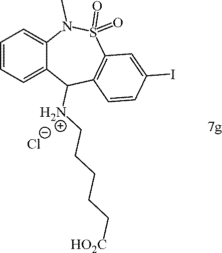 [SMILES: CN1c2ccccc2C([NH2+]CCCCCC(=O)O)c2ccc(I)cc2S1(=O)=O ; [Cl-]]

The product 7g was prepared according to the general procedure and obtained as a glassy, white foam (19.7 mg, 91%). ¹H NMR (500 MHz, CD₃OD) δ 8.37 (d, J = 1.8 Hz, 1H), 8.22 (dd, J = 8.0, 1.8 Hz, 1H), 7.76 (dd, J = 7.8, 1.2 Hz, 1H), 7.68 (d, J = 8.1 Hz, 1H), 7.66 - 7.61 (m, 1H), 7.57 (dd, J = 8.1, 1.1 Hz, 1H), 7.48 (td, J = 7.7, 1.3 Hz, 1H), 5.96 (s, 1H), 3.23 (s, 3H), 2.97 (ddd, J = 12.1, 10.4, 5.5 Hz, 1H), 2.82 (ddd, J = 12.2, 10.3, 5.9 Hz, 1H), 2.28 (t, J = 7.3 Hz, 2H), 1.77 - 1.61 (m, 2H), 1.61 - 1.54 (m, 2H), 1.39 - 1.29 (m, 2H); ¹³C NMR (126 MHz, CD₃OD) δ 177.2, 144.5, 142.5, 141.4, 137.9, 136.8, 134.8, 133.4, 129.1, 128.8, 128.5, 127.1, 98.0, 67.7, 48.2, 39.7, 34.4, 26.9, 26.7, 25.3.

### 3-(Chloro-6-methyl-5,5-dioxido-6,11-dihydrodibenzo[c,f] [1,2]thiazepin-11-yl)amino)propanoic acid hydrochloride salt (7h).

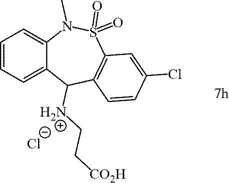 [SMILES: CN1c2ccccc2C([NH2+]CCC(=O)O)c2ccc(Cl)cc2S1(=O)=O ; [Cl-]]

The product 7h was prepared according to the general procedure and obtained as a glassy, white foam (364 mg, 98%). ¹H NMR (400 MHz, CD₃OD) δ 8.09 (d, J = 2.2 Hz, 1H), 7.94 (d, J = 8.3 Hz, 1H), 7.87 (dd, J = 8.2, 2.2 Hz, 1H), 7.77 (dd, J = 7.8, 1.4 Hz, 1H), 7.69 - 7.63 (m, 1H), 7.60 (dd, J = 8.1, 1.3 Hz, 1H), 7.49 (td, J = 7.6, 1.4 Hz, 1H), 6.02 (s, 1H), 3.30 - 3.25 (m, 1H), 3.28 (s, 3H), 3.12 (dt, J = 18.3, 6.4 Hz, 1H), 2.84 - 2.68 (m, 2H); ¹³C NMR (101 MHz, CD₃OD) δ 174.2, 142.3, 142.1, 139.0, 137.1, 135.1, 134.7, 133.6, 129.5, 129.3, 128.3, 127.8, 127.4, 68.1, 44.3, 39.4, 30.7.

### 5-(Chloro-6-methyl-5,5-dioxido-6,11-dihydrodibenzo[c,f] [1,2]thiazepin-11-yl)amino)pentanoic acid hydrochloride salt (7i).

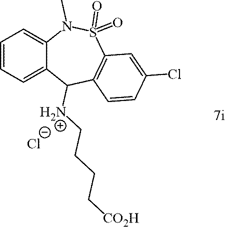 [SMILES: CN1c2ccccc2C([NH2+]CCCCC(=O)O)c2ccc(Cl)cc2S1(=O)=O ; [Cl-]]

The product 7i was prepared according to the general procedure and obtained as a glassy, off-white foam (243 mg, 98%). ¹H NMR (400 MHz, CD₃OD) δ 8.09 (d, J = 2.1 Hz, 1H), 7.92 (d, J = 8.2 Hz, 1H), 7.87 (dd, J = 8.2, 2.2 Hz, 1H), 7.74 (dd, J = 7.8, 1.4 Hz, 1H), 7.68 - 7.62 (m, 1H), 7.58 (dd, J = 8.1, 1.3 Hz, 1H), 7.49 (td, J = 7.6, 1.4 Hz, 1H), 5.98 (s, 1H), 3.24 (s, 3H), 3.00 (ddd, J = 12.3, 9.6, 5.9 Hz, 1H), 2.86 (ddd, J = 12.3, 9.6, 6.3 Hz, 1H), 2.31 (t, J = 7.0 Hz, 2H), 1.82 - 1.64 (m, 2H), 1.64 - 1.54 (m, 2H); ¹³C NMR (101 MHz, CD₃OD) δ 176.71, 142.51, 141.87, 138.84, 137.07, 135.19, 134.78, 133.50, 129.42, 129.16, 128.53, 127.88, 127.08, 67.61, 48.07, 39.71, 33.83, 26.40, 22.70.

### Example 2. Opioid Receptor Activity

The diarylthiazepinamine esters (6) and carboxylic acids (7) were tested for agonist activity at the human mu-opioid receptor (MOR) and delta-opioid receptor (DOR) using bioluminescence resonance energy transfer (BRET) assays measuring G protein activation as previously described (Table 1) (Rives, M.-L. et al. 2012; Negri, A. et al. 2013).

Transfection. Human MOR or DOR cDNA was transfected alongside G∝_{oB} with RLuc8 inserted at position 91 (G∝_{oB}-RLuc8), Gβ₁ (β₁), and Gγ₂ fused to the full-length mVenus at its N terminus (mVenus- γ2) into HEK-293T cells (5 × 10⁶ cells/plate) in 10-cm dishes using PEI (Polysciences Inc.; Warrington, PA) in a 1:1 ratio diluted in Opti-MEM (Life Technologies Corp.; Grand Island, NY) to assay for G protein activation as described previously (Rives, M.-L. et al. 2012; Negri, A. et al. 2013). Cells were maintained in Dulbecco’s Modified Eagle Medium (high glucose #11965; Life Technologies) supplemented with 10% FBS (Premium Select, Atlanta Biologicals; Atlanta, GA) and 100 U/mL penicillin and 100 µg/mL streptomycin (#15140, Life Technologies). After 24 hours the media was changed, and the experiment was performed 24 hours later (48 hours after transfection).

BRET. Transfected cells were dissociated and re-suspended in phosphate-buffered saline (PBS). Approximately 200,000 cells/well were added to a black-framed, white well 96-well plate (#60050; Perkin Elmer; Waltham, MA). The microplate was centrifuged and the cells were re-suspended in PBS. Then 5 µM of the luciferase substrate coelenterazine H was added to each well for 5 minutes. Following coelenterazine H addition, ligands were added and the BRET signal was measured at 5 minutes on a PHERAstar FS plate reader. Quantification of the BRET signal required calculating the ratio of the light emitted by the energy acceptor, mVenus (510-540 nm), over the light emitted by the energy donor, RLuc8 (485 nm). This drug-induced BRET signal was normalized using the Eₘₐₓ of [D-Ala, N-MePhe, Gly-ol]-enkephalin (DAMGO) or [D-Pen(2,5)]Enkephalin (DPDPE) as the 100% maximal response for G protein activation at MOR or DOR, respectively (Rives, M.-L. et al. 2012; Negri, A. et al. 2013). Dose response curves were fit using a three-parameter logistics equation in GraphPad Prism 6.

The following compounds listed in Table 1 activated human MOR and/or DOR. Accordingly, the compounds listed in Table 1 are agonists of MOR and/or DOR.

TABLE 1

Functional agonist activity of compounds at human MOR and DOR

| | | | |
|---|---|---|---|
| Compound | Structure | Human MOR (EC₅₀) | Human DOR (EC₅₀) |
| Tianeptine | 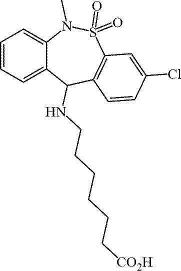 [SMILES: CN1c2ccccc2C(NCCCCCCC(=O)O)c2ccc(Cl)cc2S1(=O)=O] | 194 ± 70 nM | 37.4 ± 11.2 µM |
| 6a | 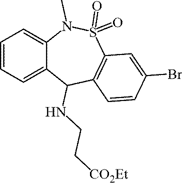 [SMILES: CCOC(=O)CCNC1c2ccccc2N(C)S(=O)(=O)c2cc(Br)ccc21] | 5.9 ± 1.0 μΜ | > 10 µM |
| 6b | 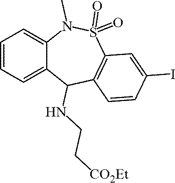 [SMILES: CCOC(=O)CCNC1c2ccccc2N(C)S(=O)(=O)c2cc(I)ccc21] | 1.6 ± 0.2 µM | > 10 µM |
| 6c | 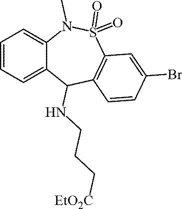 [SMILES: CCOC(=O)CCCNC1c2ccccc2N(C)S(=O)(=O)c2cc(Br)ccc21] | 270 ± 38 nM | > 10 µM |
| 6i | 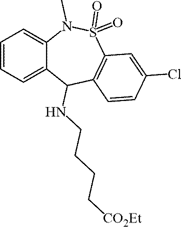 [SMILES: CCOC(=O)CCCCNC1c2ccccc2N(C)S(=O)(=O)c2cc(Cl)ccc21] | 533 ± 267 nM | > 10 μΜ |
| 6d | 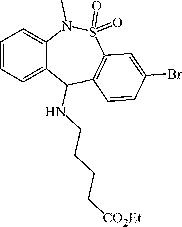 [SMILES: CCOC(=O)CCCCNC1c2ccccc2N(C)S(=O)(=O)c2cc(Br)ccc21] | 120 nM | 10 µM |
| 6e | 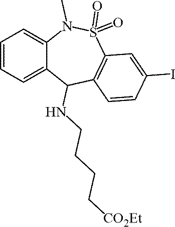 [SMILES: CCOC(=O)CCCCNC1c2ccccc2N(C)S(=O)(=O)c2cc(I)ccc21] | 28.2 ± 3.2 nM | 6.8 ± 0.03 µM |
| 6j | 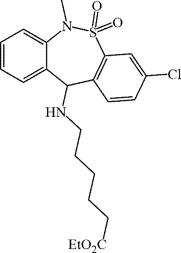 [SMILES: CCOC(=O)CCCCCNC1c2ccccc2N(C)S(=O)(=O)c2cc(Cl)ccc21] | 318 ± 162 nM | 13.9 ± 4.8 µM |
| 6f | 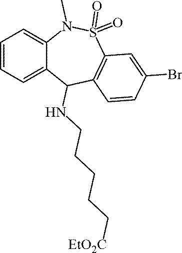 [SMILES: CCOC(=O)CCCCCNC1c2ccccc2N(C)S(=O)(=O)c2cc(Br)ccc21] | 90.8 ± 31.4 nM | 11.7 ± 0.06 µM |
| 6 g | 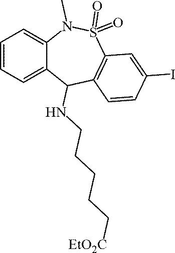 [SMILES: CCOC(=O)CCCCCNC1c2ccccc2N(C)S(=O)(=O)c2cc(I)ccc21] | 48.5 ± 4.9 nM | 8.5 ± 6.3 µM |
| 7h | 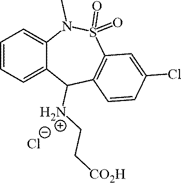 [SMILES: CN1c2ccccc2C([NH2+]CCC(=O)O)c2ccc(Cl)cc2S1(=O)=O ; [Cl-]] | 24.9 ± 12.6 µM | > 10 µM |
| 7a | 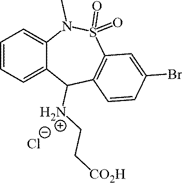 [SMILES: CN1c2ccccc2C([NH2+]CCC(=O)O)c2ccc(Br)cc2S1(=O)=O ; [Cl-]] | 4.7 ± 1.4 µM | > 10 µM |
| 7b | 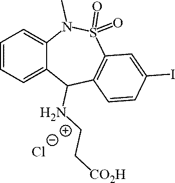 [SMILES: CN1c2ccccc2C([NH2+]CCC(=O)O)c2ccc(I)cc2S1(=O)=O ; [Cl-]] | 1.2 ± 0.3 µM | > 10 μΜ |
| 7c | 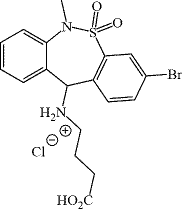 [SMILES: CN1c2ccccc2C([NH2+]CCCC(=O)O)c2ccc(Br)cc2S1(=O)=O ; [Cl-]] | 2.0 ± 0.3 µM | > 10 µM |
| 7i | 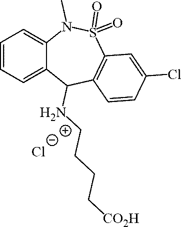 [SMILES: CN1c2ccccc2C([NH2+]CCCCC(=O)O)c2ccc(Cl)cc2S1(=O)=O ; [Cl-]] | 361.2 ± 92.9 nM | > 10 µM |
| 7d | 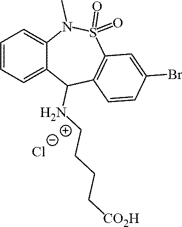 [SMILES: CN1c2ccccc2C([NH2+]CCCCC(=O)O)c2ccc(Br)cc2S1(=O)=O ; [Cl-]] | 405 nM | > 10 µM |
| 7e | 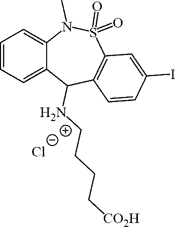 [SMILES: CN1c2ccccc2C([NH2+]CCCCC(=O)O)c2ccc(I)cc2S1(=O)=O ; [Cl-]] | 18.3 ± 0.2 nM | 8.9 ± 0.007 µM |
| 7f | 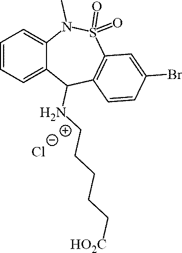 [SMILES: CN1c2ccccc2C([NH2+]CCCCCC(=O)O)c2ccc(Br)cc2S1(=O)=O ; [Cl-]] | 133 ± 13.2 nM | 23.7 ± 1.5 µM |
| 7 g | 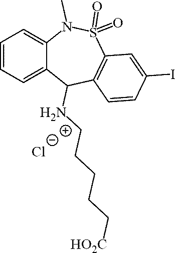 [SMILES: CN1c2ccccc2C([NH2+]CCCCCC(=O)O)c2ccc(I)cc2S1(=O)=O ; [Cl-]] | 24.3 ± 4.5 nM | 5.1 ± 1.4 µM |

Where indicated, error represents ± SEM of 2 or more independent trials. The data shows the strong trend for increasing potency as the size of the halogen substituent is increased from chloro through iodo, a trend that holds across several example subgenera

The data contained herein shows the strong trend for increasing potency as the size of the halogen substituent is increased from chloro through iodo. The compounds disclosed herein have increasing potency based on the following trend: X = Cl < X = Br < X = I. The compounds where X = I or X = Br have increased potency relative to the corresponding compounds where X = Cl.

### Example 3. Administration of MOR Agonists

An amount of any one of compounds 6a, 6b, 6c, 6d, 6e, 6f, 6g, 7a, 7b, 7c, 7d, 7e, 7f, or 7gis administered to a subject afflicted with Huntington’s disease. The amount of the compound is effective to treat the Huntington’s disease.

An amount of any one of compounds 6a, 6b, 6c, 6d, 6e, 6f, 6g, 7a, 7b, 7c, 7d, 7e, 7f, or 7g is administered to a subject afflicted with Rett syndrome. The amount of the compound is effective to treat the Rett syndrome.

An amount of any one of compounds 6a, 6b, 6c, 6d, 6e, 6f, 6g, 7a, 7b, 7c, 7d, 7e, 7f, or 7g is administered to a subject afflicted with a Rett syndrome variant. The amount of the compound is effective to treat the Rett syndrome variant.

An amount of any one of compounds 6a, 6b, 6c, 6d, 6e, 6f, 6g, 7a, 7b, 7c, 7d, 7e, 7f, or 7g is administered to a subject afflicted with Angelman syndrome or Prader-Willi syndrome. The amount of the compound is effective to treat the Angelman syndrome or the Prader-Willi syndrome.

An amount of any one of compounds 6a, 6b, 6c, 6d, 6e, 6f, 6g, 7a, 7b, 7c, 7d, 7e, 7f, or 7g is administered to a subject afflicted with CDKL5 disorder. The amount of the compound is effective to treat the CDKL5 disorder.

An amount of any one of compounds 6a, 6b, 6c, 6d, 6e, 6f, 6g, 7a, 7b, 7c, 7d, 7e, 7f, or 7g is administered to a subject afflicted with a neurological disorder, wherein the neurological disorder is neonatal onset encephalopathy; X-linked recessive mental retardation; fetal alcohol spectrum disorder; Hirschsprung disease; West syndrome; FOXG1 syndrome; or Lennox-Gastaut syndrome. The amount of the compound is effective to treat the neurological disorder.

### Example 4. Metabolism and Pharmacokinetics

Experimental Protocol for PK Studies. The brain pharmacokinetics of tianeptine and its 5-carbon metabolite (7i) were determined by Sai Life Sciences Limited (Hinjewadi, India) as follows in male C57BL/6 mice following a single administration of tianeptine. A group of male mice were administered with a solution formulation of tianeptine (normal saline with 7.5% NMP and 5% Solutol HS) intraperitoneally at a dose of 30 mg/kg. Brain samples were collected from three mice at 0.08, 0.25, 0.5, 1, 2, 4, 8 and 24 h, homogenized using ice-cold phosphate buffer saline (pH 7.4), and stored below -70° C. until analysis. Total homogenate volume was three times the tissue weight. All samples were processed for analysis by protein precipitation using acetonitrile (ACN) and analyzed with a fit-for-purpose LC/MS/MS method (LLOQ - 1.01 ng/mL in brain). Pharmacokinetic parameters were calculated using the non-compartmental analysis tool of Phoenix WinNonlin® (Version 6.3).

Tianeptine is known to be metabolized primarily by β-oxidation of the carboxylic acid side chain, in a manner similar to fatty acids (Grislain, L. et al. 1990). This results in metabolites with shortened carboxylic acid side chains. In the specific case of tianeptine, these metabolites include compounds 7h and 7i. Other carboxylic acid analogs, including the compounds described in the present disclosure, can be metabolized in a similar manner. Surprisingly, the 5-carbon metabolite (7i) resulting from degradation of tianeptine exhibits a significantly longer half-life and greater brain exposure (as measured by AUC) compared to the parent compound (Table 2). Accordingly, the shortened carboxylic acid analogs of this application (where X is Br or I), in addition to their higher potency, can exhibit improved pharmacokinetics, and thus enhanced therapeutic efficacy over tianeptine and analogs of tianeptine where Cl is replaced with Br or I. Further, the specific length of the side chain selected is useful for fine control of the pharmacokinetic profile in this genus.

TABLE 2

Pharmacokinetic parameters of tianeptine and the resulting 5-carbon metabolite (7i) in the brains of C57BL/6 mice following a single injection (30 mg/kg, i.p.) of tianeptine

| Compound | Route | Dose (mg/kg) | Matrix | Tₘₐₓ (h) | Cₘₐₓ (ng/g) | AUCₗₐₛₜ (h*ng/g) | T_{½} (h) | CL (mL/min/kg) |
|---|---|---|---|---|---|---|---|---|
| tianeptine | i.p. | 30 | brain | 0.08 | 1019 | 351 | 0.29 | 1417 |
| 7i | - | - | brain | 0.5 | 2865 | 5963 | 2.33 | 78 |

### Example 5. Esters as Prodrugs

Carboxylate esters are well known as prodrugs for the corresponding carboxylic acids obtained by hydrolysis (Beaumont, et al. 2003). Such ester prodrugs show improved oral bioavailability, better brain penetration, and/or longer duration of action compared to their carboxylic acid counterparts. Accordingly, compounds of this application having an ester side chain (6), although biologically active on their own, may also act as prodrugs for the corresponding carboxylic acids (7). Further, one skilled in the art will be able to apply the methods and knowledge of this application to prepare additional prodrugs. For example, the type of ester (e.g., methyl, ethyl, propyl, isopropyl, tert-butyl, phenyl) or the length of the side chain may be varied to adjust the activity and pharmacokinetic properties of the prodrugs and their corresponding carboxylic acid hydrolysis products.

### Example 6. Combinations With NMDA Receptor Antagonists

Antagonists of the N-methyl-D-aspartate receptor (NMDAR) are known to potentiate the beneficial effects of opioid receptor agonists (Trujillo, K.A. et al. 1994; Mao, J. et al. 1996). Specific NMDAR antagonists are known to be effective therapeutics (Murrough, J.W. et al. 2013). Therefore, pharmaceutical compositions of the compounds disclosed herein, combined with NMDAR antagonists, are useful in the treatment of neurological disorders, as described herein. Alternatively, the opioid modulator and NMDAR antagonist may be dosed separately, as a novel method for treating neurological disorders.

The pharmaceutical compositions of the compounds of the present disclosure, combined with NMDAR antagonists, are useful in the treatment of neurological disorders, as set out herein. The NMDAR antagonist can serve as an adjunct to prevent the development of tolerance to the compounds of the present disclosure with chronic use.

NMDAR antagonists are also known to be effective therapeutic agents (Murrough, J.W. et al. 2013; Zarate, C.A. Jr et al. 2006). Therefore, pharmaceutical compositions of the compounds of the present disclosure, combined with NMDAR antagonists, may be used to treat neurological disorders with enhanced efficacy compared to the compounds of the present disclosure alone. Alternatively, the opioid modulator and NMDAR antagonist may be dosed separately, as a novel method for treating the disorders described herein.

### Non-Limiting Examples of NMDA Receptor Antagonists:

Dextromorphinans - dextromethorphan, dextrorphan, dextrallorphan
Adamantanes - memantine, amantadine, rimantadine, nitromemantine (YQW-36)
Arylcyclohexylamines - ketamine (and its analogs, e.g., tiletamine), phencyclidine (and its analogs, e.g., tenocyclidine, eticyclidine, rolicyclidine), methoxetamine (and its analogs), gacyclidine (GK-11);
Miscellaneous - neramexane, lanicemine (AZD6765), diphenidine, dizocilpine (MK-801), 8a-phenyldecahydroquinoline (8A-PDHQ), remacemide, ifenprodil, traxoprodil (CP-101,606), eliprodil (SL-82.0715), etoxadrol (CL-1848C), dexoxadrol, WMS-2539, NEFA, delucemine (NPS-1506), aptiganel (Cerestat; CNS-1102), midafotel (CPPene; SDZ EAA 494), dexanabinol (HU-211 or ETS2101), selfotel (CGS-19755), 7-chlorokynurenic acid (7-CKA), 5,7-dichlorokynurenic acid (5,7-DCKA), L-683344, L-689560, L-701324, GV150526A, GV196771A, CERC-301 (formerly MK-0657), atomoxetine, LY-235959, CGP 61594, CGP 37849, CGP 40116 (active enantiomer of CG 37849), LY-233536, PEAQX (NVP-AAM077), ibogaine, noribogaine, Ro 25-6981, GW468816, EVT-101, indantadol, perzinfotel (EAA-090), SSR240600, 2-MDP (U-23807A), AP-7

### Example 7. Combinations With NMDA Receptor Partial Agonists

Weak partial agonists of NMDAR are also known (Moskal, J.R. et al. 2005), and can be used to produce beneficial or synergistic effects similar to an antagonist when intrinsic glutamate signaling activity is high or over-activated. Therefore, pharmaceutical compositions of the novel compounds disclosed herein, combined with NMDAR partial agonists, are useful in the treatment of neurological disorders, as set out in this disclosure. Alternatively, the opioid modulator and NMDAR partial agonist may be dosed separately, as a novel method for treating neurological disorders.

Pharmaceutical compositions of the compounds of the present disclosure, combined with NMDAR partial agonists, may be useful in the treatment of neurological disorders, as set out herein. The NMDAR partial agonist can serve as an adjunct to prevent the development of tolerance to the compounds of the present disclosure with chronic use. Similarly, pharmaceutical compositions of the compounds of the present disclosure, combined with NMDAR partial agonists, may be used to treat neurological disorders with enhanced efficacy compared to the compounds of this disclosure, taken alone. Alternatively, the opioid modulator and NMDAR partial agonist may be dosed separately, as a novel method for treating the disorders described herein.

### Non-Limiting Examples of NMDA Receptor Partial Agonists:

NRX-1074, rapastinel (GLYX-13)

### Example 8. Combinations With Neurokinin 1 Receptor Antagonists

Antagonists of the neurokinin 1 receptor (NK-1) are known to modulate the effects of opioid agonists. More specifically, NK-1 antagonists attenuate opioid agents in animal models (Robinson, J.E. et al. 2012). NK-1 antagonists are also known to be effective therapeutic agents (Kramer, M.S. et al. 2004). Therefore, pharmaceutical compositions of the novel compounds disclosed herein, combined with NK-1 antagonists, are useful in the treatment of neurological disorders, as set out herein, with increased efficacy and less potential for abuse.

Alternatively, the opioid modulator and NK-1 antagonist may be dosed separately, as a novel method for treating neurological disorders.

Pharmaceutical compositions of the compounds of the present disclosure, combined with NK-1 antagonists, are useful in the treatment of neurological disorders, as set out herein. The NK-1 antagonist can serve as an adjunct to reduce the abuse potential of the compounds of the present disclosure. NK-1 antagonists are also known to be effective therapeutic agents (Kramer, M.S. et al. 2004). Therefore, pharmaceutical compositions of the compounds of the present disclosure, combined with NK-1 antagonists, can be used to treat neurological disorders with enhanced efficacy compared to the compounds of the present disclosure alone. Alternatively, the opioid modulator and NK-1 antagonist may be dosed separately, as a novel method for treating the neurological disorders, as set out herein.

Non-Limiting Examples of Neurokinin 1 Receptor Antagonists: aprepitant, fosaprepitant, casopitant, maropitant, vestipitant, vofopitant, lanepitant, orvepitant, ezlopitant, netupitant, rolapitant, L-733060, L-703606, L-759274, L-822429, L-760735, L-741671, L-742694, L-732138, CP-122721, RPR-100893, CP-96345, CP-99994, TAK-637, T-2328, CJ-11974, RP 67580, NKP608, VPD-737, GR 205171, LY686017, AV608, SR140333B, SSR240600C, FK 888, GR 82334

### Example 9. Combinations With Neurokinin 2 Receptor Antagonists

Antagonists of the neurokinin 2 receptor (NK-2) are known to show therapeutic effects (Overstreet, D.H. et al. 2010). Therefore, pharmaceutical compositions of the novel compounds disclosed herein, combined with NK-2 antagonists, are useful in the treatment of neurological disorders, as set out herein, with increased efficacy. Alternatively, the opioid modulator and NK-2 antagonist may be dosed separately, as a novel method for treating neurological disorders.

Pharmaceutical compositions of the compounds of the present disclosure, combined with NK-2 antagonists, are useful in the treatment of neurological disorders, as set out herein, with increased efficacy compared to the compounds of the present disclosure alone. Alternatively, the opioid modulator and NK-2 antagonist may be dosed separately, as a novel method for treating neurological disorders.

### Non-Limiting Examples of Neurokinin 2 Receptor Antagonists:

saredutant, ibodutant, nepadutant, GR-159897, MEN-10376

### Example 10. Combinations With Neurokinin 3 Receptor Antagonists

Antagonists of the neurokinin 3 receptor (NK-3) are known to show therapeutic effects (Salome, et al. 2006). Further, the actions of NK-3 modulators show a dependency on the opioid receptor system (Panocka, I. et al. 2001). Therefore, pharmaceutical compositions of the novel compounds disclosed herein, combined with NK-3 antagonists, are useful in the treatment of neurological disorders, as set out herein, with increased efficacy. Alternatively, the opioid modulator and NK-3 antagonist may be dosed separately, as a novel method for treating neurological disorders.

Pharmaceutical compositions of the compounds of the present disclosure, combined with NK-3 antagonists, may be useful in the treatment of neurological disorders, as set out herein, with increased efficacy compared to the compounds of the present disclosure alone. Alternatively, the opioid modulator and NK-3 antagonist may be dosed separately, as a novel method for treating neurological disorders.

### Non-Limiting Examples of Neurokinin 3 Receptor Antagonists:

osanetant, talnetant, SB-222200, SB-218795

### Example 11. Combinations With DOR Agonists

DOR Agonists have also been shown to elicit therapeutic effects (Torregrossa, et al. 2005). Therefore, pharmaceutical compositions of the novel compounds disclosed herein, combined with DOR agonists, are useful in the treatment of neurological disorders, as set out herein, with increased efficacy. Alternatively, the opioid modulator and DOR agonist may be dosed separately, as a novel method for treating neurological disorders.

DOR Agonists have been shown to elicit therapeutic effects (Saitoh, A. et al. 2004; Torregrossa, et al. 2005; Jutkiewicz, E.M. 2006; Vanderah, T.W. 2010; Peppin, J.F. and Raffa, R.B. 2015). They have also been shown to reverse the respiratory depression induced by MOR agonists (Su, Y-F. et al. 1998). Therefore, pharmaceutical compositions of the compounds of the present disclosure, combined with DOR agonists, are useful in the treatment of neurological disorders, as set out herein, with increased efficacy or reduced side effects compared to the compounds of the present disclosure alone. Alternatively, the opioid modulator and DOR agonist may be dosed separately, as a novel method for treating the neurological disorders described herein.

### Non-Limiting Examples of DOR Agonists:

tianeptine, (+)BW373U86, SNC-80, SNC-121, SNC-162, DPI-287, DPI-3290, DPI-221, TAN-67, KN-127, AZD2327, JNJ-20788560, NIH11082, RWJ-394674, ADL5747, ADL5859, UFP-512, AR-M100390, SB-235863, 7-spiroindanyloxymorphone.

### Example 12. Combinations With DOR Antagonists

DOR antagonists have been shown in animals to attenuate several negative side effects exhibited by MOR agonists. For example, DOR antagonists slow the development of tolerance to the analgesic effects of morphine and also limit physical dependence (Hepburn, M.J. et al. 1997; Suzuki, T. et al. 1997). Further, DOR antagonists have been shown to attenuate the abuse liability of MOR agonists as determined by both conditioned place preference and self-administration paradigms (Suzuki, T. et al. 1994; Martin, T.J. et al. 2000). Therefore, pharmaceutical compositions of the compounds of the present disclosure, combined with DOR antagonists, may be useful in the treatment of neurological disorders, as set out herein. The DOR antagonist can serve as an adjunct to prevent the development of tolerance to the compounds of the present disclosure or to reduce their abuse potential. Additionally, DOR antagonists have been shown to reverse the respiratory depression induced by MOR agonists (Su, Y-F. et al. 1998). Therefore, pharmaceutical compositions of the compounds of the present disclosure, combined with DOR antagonists, are useful in the treatment of neurological disorders, as set out herein, with enhanced efficacy or safety compared to the compounds of the present disclosure alone. Alternatively, the opioid modulator and DOR antagonist may be dosed separately, as a novel method for treating the neurological disorders described herein.

### Non-Limiting Examples of DOR Antagonists:

naltrindole, naltriben, N-benzylnaltrindole, 7-benzylidenenaltrexone, SDM25N, DPI-2505

### Example 13. Combinations With SSRI or SNRIs

Selective serotonin reuptake inhibitors (SSRIs) and serotonin-norepinephrine reuptake inhibitors (SNRIs) are effective therapeutic agents (Thase, M.E. 2008; Vaswani, M. et al. 2003; Marks, D.M. et al. 2009). Therefore, pharmaceutical compositions of the compounds of the present disclosure, combined with SSRIs or SNRIs, are useful in the treatment of neurological disorders, as set out herein, with increased efficacy compared to the compounds of the present disclosure alone. Alternatively, the opioid modulator and SSRI or SNRI may be dosed separately, as a novel method for treating the neurological disorders described herein. Further, the compounds of the present disclosure may be used as an add-on therapy to enhance the efficacy of preexisting SSRI or SNRI therapy for the disorders described herein.

### Non-Limiting Examples of SSRIs:

citalopram, escitalopram, fluoxetine, fluvoxamine, paroxetine, sertraline, dapoxetine

### Non-Limiting Examples of SNRIs:

venlafaxine, desvenlafaxine

### Example 14. Combination With Naloxone

Naloxone is an MOR antagonist that is effective in blockading all behavioral effects induced by classical MOR agonists and is the standard treatment for opioid overdose. It is highly bioavailable by parenteral routes of administration but not by the oral route (Smith, K. et al. 2012). Accordingly, pharmaceutical compositions containing mixtures of an MOR agonist and naloxone remain effective when given by the oral route but the naloxone component inhibits the effects of the MOR agonist component when the mixture is administered parenterally. Thus, addition of naloxone to pharmaceutical compositions containing MOR agonists is useful for preventing their misuse or abuse by parenteral routes of administration. Therefore, pharmaceutical compositions of the compounds of the present disclosure, combined with naloxone, are useful in providing the therapeutic benefits of the compounds of the present disclosure while having diminished potential for abuse.

### Example 15. Combinations With Methylnaltrexone

Constipation is a frequent, unpleasant side effect of MOR agonists resulting from inhibition of intestinal smooth muscle contractions via activation of MORs located in this tissue. Methylnaltrexone (Relistor) is a clinically approved quaternary ammonium salt of the opioid receptor antagonist naltrexone that does not cross the blood brain barrier. Accordingly, this compound is capable of inhibiting MORs in the gastrointestinal tract and preventing opioid-induced constipation while avoiding simultaneous inhibition of centrally mediated therapeutic effects. Therefore, pharmaceutical compositions of the compounds of the present disclosure, combined with methylnaltrexone, are useful in the treatment of neurological disorders, as set out herein, with reduced constipation compared to the compounds of the present disclosure alone. Alternatively, the opioid modulator and methylnaltrexone may be dosed separately, as a novel method for treating the neurological disorders described herein with less constipation.

### Example 16. Efficacy of Compounds in Treating Huntington’s Disease (HD)

Overview - The compounds of the present invention were tested for their potential ability to reverse a Huntington’s disease-like behavioral phenotype in mice using the SmartCube® (SC) system and associated data analysis methods. The tested compounds demonstrated activity likely to reverse the disease phenotype and therefore, are useful in treating Huntington’s disease and other neurological disorders, as described herein.

Animals - All mouse husbandry and experimental procedures were conducted with the approval of the appropriate Animal Care and Use Committee. Q175 HD model heterozygous mice (PsychoGenics) were bred and aged. These mice were subjected to testing at 6 and 10 months of age in the SC system to establish a Huntington’s Disease-like behavioral phenotype for comparison to drug-induced behavioral phenotypes, as described further below. For screening of the test compounds, wild-type male C57BL/6 mice were used. All animals were examined, handled, and weighed prior to initiation of testing to assure adequate health and suitability and to minimize non-specific stress associated with manipulation. During the course of the study, mice were group-housed in OPTI mouse ventilated cages with 4 mice/cage. 12/12 light/dark cycles were maintained. The room temperature was maintained between 20 and 23° C. with a relative humidity maintained between 30 and 70%. Chow and water were provided ad libitum for the duration of the study. Animals were acclimated to the vivarium for at least one week prior to commencing testing and tested at 8-9 weeks of age. Body weight was measured prior to testing.

Treatment groups - 12 mice were used in each of the following groups:
1. Vehicle
2. compound 84 1.5 mg/kg
3. compound 84 3.0 mg/kg
4. compound 84 6.0 mg/kg
5. compound 84 12 mg/kg
6. compound 7e 2.25 mg/kg
7. compound 7e 4.5 mg/kg
8. compound 7e 9.0 mg/kg
9. compound 7e 18 mg/kg
10. tianeptine, 10 mg/kg
11. tianeptine, 20 mg/kg

Compound 84:

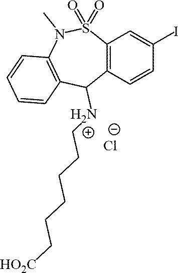 [SMILES: CN1c2ccccc2C([NH2+]CCCCCCC(=O)O)c2ccc(I)cc2S1(=O)=O ; [Cl-]]

Compound 7e:

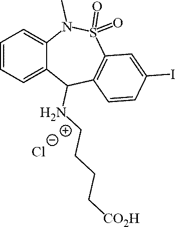 [SMILES: CN1c2ccccc2C([NH2+]CCCCC(=O)O)c2ccc(I)cc2S1(=O)=O ; [Cl-]]

Administration of test compounds was by subcutaneous injection at an injection volume of 10 mL/kg, except for tianeptine, which was administered intraperitoneally at an injection volume of 10 mL/kg. Saline was used as the vehicle for all test compounds. All testing and data analysis was carried out blinded to treatment.

Behavior testing - The SC system (PsychoGenics) was utilized to measure numerous spontaneous behaviors and response to challenges in the same testing environment. The hardware included force sensors and a number of aversive stimuli to elicit behavior. Three high-resolution video cameras provided a constant 3D view of the mouse in the SC apparatus throughout the entire testing period. The mice were exposed to a sequence of challenges during the 45-minute test period. Mice were injected with vehicle or test compound and placed in the SC 15 minutes after administration. The cubes were cleaned between each run. Computational modelling was performed for behavioral signatures, including tail flick, grooming/sniffing, ear twitch (orientation response), and orientation (walking, running, rearing, freezing).

Data analysis - Several analytical methods including Bayesian probabilistic density models were utilized alongside data mining algorithms (Alexandrov, V. et al. 2015). The algorithms can consider more than 2,000 measures including frequency and duration of behavioral states such as grooming, rearing, mobility, behavioral transitions and many other features obtained during the test session.

Two major types of analyses are routinely conducted: class and subclass. For class and subclass analysis, a reference data set was built from hundreds of drug doses in multiple drug classes plus a control group. Each reference drug was tested at multiple doses appropriate for that drug in mice. The best performing classifiers were chosen from evaluation tests and two separate types of classifiers were built that make independent predictions at drug class and subclass levels.

The class consists of drugs that are currently on the market or that have been clinically validated for that specific indication. The subclass consists of both marketed drugs and other compounds that have been mechanistically validated and is a larger set than the class. The reference databases have been continually expanded and new databases have been kept in development. Once validated, these new databases were incorporated into the analysis to provide additional information about each compound tested.

Data from the screening was processed using proprietary computer vision and data mining algorithms developed by PsychoGenics and the results were compared to signatures of the reference compounds in the database. Multiple analyses of the data were performed to quantitatively produce independent predictions of drug class, and drug subclass. The behavioral signatures of the test drugs were evaluated using these classifiers to confirm therapeutic utility. The results for the class and subclass analyses were presented as standardized bar charts with percentages that sum to 100 for each dose. The results of the classification at the drug level were presented as individual similarities.

Similarity analysis - The outcome from SC provides a large set of features (behavioral parameters) that has been used for various analyses. Many of these features have been correlated. Therefore, it has been useful to form statistically independent combinations of the original features (further referred to as de-correlated features). Each de-correlated feature extracted information from the whole cluster of original features, so the new feature space had lower dimensionality. Next, a feature ranking algorithm was applied to score each feature for its discrimination power (ability to separate the two groups, e.g., reference and test).

Ranking was used in the analyses because it weighs each feature change by its relevance. If there were to be a significant change in some irrelevant features measured for a particular phenotype, the low rank of these features will automatically reduce the effect of such change in the analyses. This means that a conventional “feature selection” approach did not need to be used and information was not lost among the less informative features. Ranking was applied to either the original or the de-correlated features.

It was also useful to examine the de-correlated ranked features as “clouds”. This refers to Gaussian distributions approximating the groups of mice (e.g., between test compound and vehicle or between test compound and a reference compound like tianeptine) in the ranked de-correlated features space. This was used to calculate a quantitative measure of separability (distinguishability) between the two groups. The two highest ranked de-correlated features were chosen to form the 2D coordinate plane for visualization purposes. For visualization purposes, it was possible to plot each cloud with its semi-axes equal to one standard deviation along the corresponding dimensions.

In “proximity-to-the-drug” experiments, the data are typically presented as three classes: reference, target, and test drug. Therefore, it was deemed instructive to consider and plot the third group, test drug, in the same coordinate system that best discriminates the other groups (reference and target). The drug treatment effect was then able to be represented as a combination of two components: one along the direction of the “proximity line” (the line connecting the centers of the target and reference clouds) and the component orthogonal to (“pointing away” from) that direction. The relative length of the “proximity” line with respect to the target-reference distance was then able to be interpreted as the “proximity to target”, whereas the relative length of the “other effect” line represents feature changes that move the target+treatment group away from the reference group. The summary of this analysis was effectively represented as a bar graph, which was referred to as the proximity signature.

Drug-induced behavior similarity analysis (DBSA) - Computational methods for in vivo drug discovery using large-scale datasets have been used to substantially reduce the high cost of drug screening by providing comprehensive and systematic means to discover new biological targets for existing drugs. One of the most successful computational methods for drug screening has been the correlative analysis, which uses a wide collection of genome-wide transcriptional expression data from cultured human cells or in vivo behavioral data following treatment with bioactive small molecules together with pattern-matching algorithms to discover functional connections between drugs and diseases.

The underlying hypothesis for correlative analysis is that a drug effective in the treatment of a disease (or reduction of side-effects of another compound) should induce an opposite behavioral profile to that seen in the un-treated disease state (or the other compound), whereas drugs with similar therapeutic indications should induce similar behavioral profiles. Based on this principle, an algorithm called Drug Behavioral Signature Analysis (DBSA; PsychoGenics) was employed. This has allowed drug-induced behavioral data to be used to predict functional connections among drugs or between drugs and diseases.

DBSA was used to compare the behavioral profile induced by a test drug in the SC to the behavioral profile of the disease model of interest in the SC and to compute a similarity score to identify the potential therapeutic benefit of the test drug. This analysis can thereby identify compounds with three different sets of profiles: a) ‘similar’ indicating a profile similar to that of the test compound or disease; b) ‘reverse’ indicating a profile opposite to that of the test compound or disease, thus suggesting a potential therapeutic effect on the disease; and c) ‘random’ indicating no similarity. The profile of any desired compound or disease can be queried against an existing database of compounds and disease states in order to identify compounds with similar or reverse profiles, depending on the underlying requirements.

For DBSA analysis in the present experiment, the SC behavioral profile induced by each test compound in wild-type mice was compared to the SC behavioral profile of Q175 heterozygous mice (a mouse model of Huntington’s disease) at 6 and 10 months of age. Results of DBSA are presented as normalized enrichment scores (the sign +/- of which indicates similar/reverse signature), associated p-value (signifying random or non-random), and odds-ratio (level of enrichment).

Additional information on data analysis with the SC can be found in Alexandrov, V. et al. 2015, the contents of which are herein incorporated by reference.

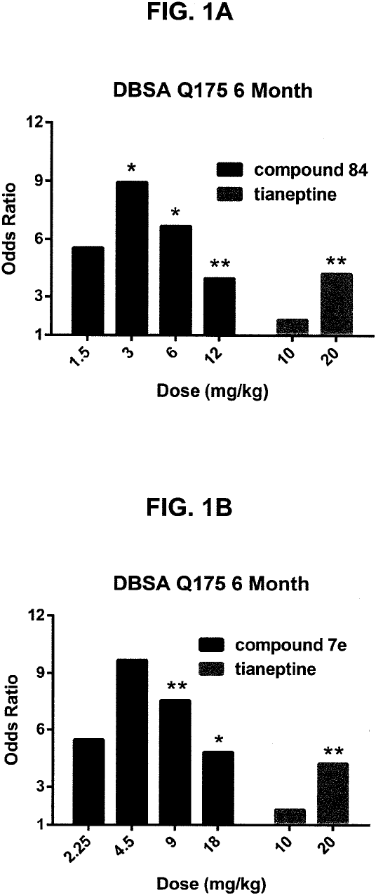

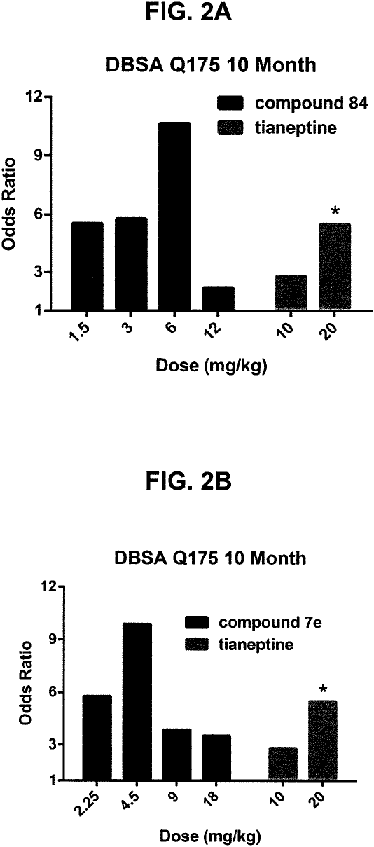

Results - Administration of the disclosed compounds to wild-type mice induced a behavioral phenotype opposed to the behavioral phenotype exhibited by Q175 mice at 6 and 10 months of age, as demonstrated by DBSA analysis (FIGS. 1A-2B), indicating that the compounds can be used to reverse the Huntington’s disease-like behavioral deficits exhibited by Q175 mice. The compounds are therefore expected to be beneficial in the treatment of Huntington’s disease and other neurological disorders, as described herein. Comparison to tianeptine in the same experimental paradigm also revealed that the disclosed compounds are more potent and more efficacious than this commercially available pharmaceutical agent.

### DISCUSSION

The compounds disclosed herein activate MOR or dually activate MOR and DOR. Furthermore, this activity leads to indirect modulation of the glutamatergic system. Accordingly, the compounds disclosed herein are also useful as neurological therapeutics.

Compounds of the present disclosure are advantageous compared to tianeptine due to their higher potency, which allows reduced dosing to treat a subject and thus, less probability for idiosyncratic toxicity due to off target effects.

The compounds of the present disclosure are also advantageous compared to tianeptine in that they are longer lasting in vivo (longer half-life). Accordingly, the compounds of the present disclosure can replace tianeptine therapy with reduced side effects and reduced dosing frequency, i.e., one or two times daily as compared to three times daily for tianeptine.

An additional aspect of the disclosure provides synthetic methods and chemical intermediates that may be used to encompass chemical space around the diarythiazepinamine core structure.

### REFERENCES

Alexandrov, V.; Brunner, D.; Hanania, T.; Leahy, E. European Journal of Pharmacology 2015, 753, 127-134.

Barbier, E. et al. Neuropsychopharmacology 2013, 38, 976-984.

Bart, G.J. Addict. Dis. 2012, 31, 207-25.

Beaumont, K.; Webster, R.; Gardner, I.; Dack, K. Curr. Drug Metab. 2003, 4, 461-485.

Berrocoso, E.; Sánchez-Blázquez, P.; Garzón, J.; Mico, J.A. Curr.Pharm. Des. 2009, 15, 1612-1622.

Besson, A.; Privat, A.M.; Eschalier, A.; Fialip, J.Psychopharmacology 1996, 123, 71-78.

Bodkin, J.A.; Zornberg, G.L.; Lukas, S.E.; Cole, J.O. J. Clin. Psychopharmacol. 1995, 15, 49-57.

Buchwald, S.L.; Huang, X.; Zim, D. Ligands for metals and improved metal-catalyzed processes based thereon. US2004/171833 A1, 2004.

Corbett, A. D.; Henderson, G.; McKnight, A.T.; Paterson, S.J.; Brit. J. Pharmacol. 2006, 147, S153-S162.

David, D.J. et al. Neuron 2009, 62, 479-493.

Dreher, S.D.; Lim, S.-E.; Sandrock, D. L.; Molander, G.A. J. Org. Chem. 2009, 74, 3626-3631.

Durand, P.; Richard, P.; Renaut, P. J. Org. Chem. 1998, 63, 9723-9727.

Gassaway, M.M.; Rives, M-L.; Kruegel A.C.; Javitch, J.A.; Sames, D. Transl. Psychiatry 2014, 4, e411.

Gilleron, P.; Wlodarczyk, N.; Houssin, R.; Farce, A.; Laconde, G.; Goossens, J.-F.; Lemoine, A.; Pommery, N.; Hénichart, J.-P.; Millet, R. Bioorg. Med. Chem. Lett. 2007, 17, 5465-5471.

Grislain, L.; Gele, P.; Bertrand, M.; Luijten, W.; Bromet, N.; Salvadori, C.; Kamoun, A. Drug Metab. Dispos. 1990, 18, 804-808.

Hepburn, M.J. et al. J. Pharmacol. Exp. Ther. 1997, 281, 1350-1356.

Invernizzi, R.; Pozzi, L.; Garattini, S.; Samanin, R. Neuropharmacology 1992, 31, 221-227.

Jutkiewicz, E.M. Mol. Interv. 2006, 6, 162-169.

Kramer, M. S. et al. Neuropsychopharmacology 2004, 29, 385-392.

Mao, J.; Price, D. D.; Caruso, F. S.; Mayer, D. J. Pain 1996, 67, 361-368.

Martin, T.J. et al. J. Pharmacol. Exp. Ther. 2000, 294, 975-982.

McNeill, E.; Barder, T. E.; Buchwald, S. L. Org. Lett. 2007, 99, 3785-3788.

Moskal, J. R. Et al. Neuropharmacology 2005, 49, 1077-1087.

Murrough, J. W.; Iosifescu, D. V.; Chang, L. C.; Al Jurdi, R. K.; Green, C. E.; Perez, A. M.; Iqbal, S.; Pillemer, S.; Foulkes, A.; Shah, A.; Charney, D. S.; Mathew, S. J. Am. J. Psychiatry 2013, 170, 1134-1142.

Negri, A. Et al. J. Chem. Inf. Model.2013, 53 (3), 521-526.

Overstreet, D.H. Et al. Pharmacol. Biochem. Behav.2010, 96, 206-210.

Rives, M.-L. et al. J. Biol.Chem. 2012, 287 (32), 27050-27054.

Robinson, J. E. et al. Psychopharmacology 2012, 220, 215-224.

Pan, J.; Wang, X.; Zhang, Y.; Buchwald, S. L. Org. Lett. 2011, 13, 4974-4976.

Panocka, I. et al. Peptides 2001, 22, 1037-1042.

Pattinson, K.T.S. Br. J. Anaesth. 2008, 100, 747-758.

Paul, I.A.; Skolnick, P. Ann. N. Y. Acad. Sci. 2003, 1003, 250-272.

Peppin, J.F.; Raffa, R.B. J. Clin. Pharm. Ther. 2015, 40, 155-166.

Prossin, A.R.; Love, T.M.; Koeppe, R.A.; Zubieta, J-K.; Silk, K.R. Am. J. Psychiatry 2010, 167, 925-933.

Saitoh, A. et al. J. Pharmacol. Sci. 2004, 95, 374-380.

Salomé, N.; Stemmelin, J.; Cohen, C.; Griebel, G. Pharmacol. Biochem. Behav. 2006, 83, 533-539.

Sansone, R.A.; Whitecar, P.; Wiederman, M.W. Int. J. Psychiatry Med. 2008, 38, 217-226.

Smith, K. et al. Int. J. Clin. Pharmacol. Ther. 2012, 50, 360-367.

Su, Y.F.; McNutt, R.W.; Chang, K.J. J. Pharmacol. Exp. Ther. 1998, 287, 815-823.

Suzuki, T. et al. Jpn. J. Pharmacol. 1994, 66, 131-137.

Suzuki, T. et al. PharmacolBiochem. Behav. 1997., 57, 293-299.

Svoboda, K.R.; Adams, C.E.; Lupica, C.R.; J. Neurosci. 1999, 19, 85-95.

Thase, M.E. Psychopharmacol. Bull. 2008, 41, 58-85.

Torregrossa, M.M. et al. Psychopharmacology (Berl). 2005, 183(1), 31-40.

Trujillo, K. A.; Akil, H. Brain Res. 1994, 633, 178-188.

Uy, R.; Yang, L.; Zhou, H.; Price, S.C.; You, W. Macromolecules 2011, 44, 9146-9154.

Vanderah, T.W. Clin. J. Pain. 2010, 26 Suppl, S10-15.

Vaswani, M.; Linda, F.K.; Ramesh, S. Prog. Neuropsychopharmacol. Biol. Psychiatry 2003, 27, 85-102.

Wermeling, D.P. Ther. Adv. Drug Saf. 2015, 6, 20-31.

Williams, J. T. et al. Pharmacol. Rev. 2013, 65, 223-254.

Xie, C.W., Lewis D.V. J. Neurophysiol. 1997, 78: 759-766.

Zarate, C.A. Jret al. Arch. Gen. Psychiatry 2006, 63, 856-864.

Persons of ordinary skill can utilize the disclosures and teachings herein to produce other embodiments and variations without undue experimentation. All such embodiments and variations are considered to be part of this invention.

Accordingly, one of ordinary skill in the art will readily appreciate from the disclosure that later modifications, substitutions, and/or variations performing substantially the same function or achieving substantially the same result as embodiments described herein may be utilized according to such related embodiments of the present invention. Thus, the invention is intended to encompass, within its scope, the modifications, substitutions, and variations to processes, manufactures, compositions of matter, compounds, means, methods, and/or steps disclosed herein.

The description herein may contain subject matter that falls outside of the scope of the claimed invention. This subject matter is included to aid understanding of the invention.

In this specification, where reference has been made to external sources of information, including patent specifications and other documents, this is generally for the purpose of providing a context for discussing the features of the present invention.

What is claimed is:

1. A compound having the structure: 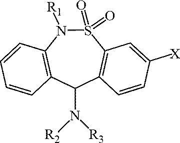
wherein
R₁ is —H or -(alkyl);
R₂ is -(alkyl)-CO₂H or -(alkyl)-CO₂-(alkyl);
R₃ is —H or -(alkyl); and
X is —Br or —I,
or a pharmaceutically acceptable salt or ester thereof,
wherein the compound is for treating or preventing a neurological disorder in a subject, wherein the neurological disorder is selected from Huntington’s disease; Rett syndrome; a Rett syndrome variant; Angelman syndrome; Prader-Willi syndrome; neonatal onset encephalopathy; X-linked recessive mental retardation; fetal alcohol spectrum disorder; Hirschsprung disease; CDKL5 disorder; West syndrome; FOXG1 syndrome; or Lennox-Gastaut syndrome.

2. The compound of claim 1, wherein
R₁ is —H or -(alkyl);
R₂ is -(C₂-C₅ alkyl)-CO₂H or -(C₂-C₅ alkyl)-CO₂-(alkyl);
R₃ is —H or -(alkyl); and
X is —Br or —I.

3. The compound of claim 2, wherein
R₁ is —H or -(alkyl);
R₂ is -(C₂-C₅ alkyl)-CO₂H;
R₃ is —H or -(alkyl); and
X is —Br or —I.

4. The compound of claim 1, wherein
R₁ is —CH₃; and
R₃ is —H.

5. The compound of claim 1, wherein
R₁ is —H, —CH₃ or —CH₂CH₃; and R₃ is —H, —CH₃ or —CH₂CH₃.

6. The compound of claim 1, wherein
R₁ is —CH₃; and
R₃ is —H.

7-9. (canceled)

10. The compound of claim 1 having the structure: 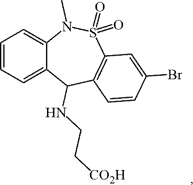 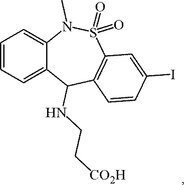 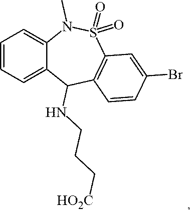 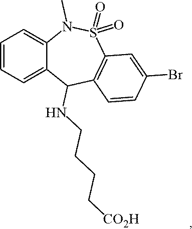 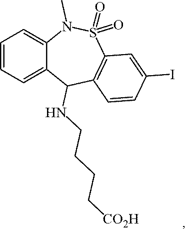 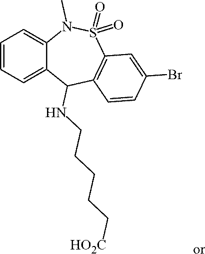 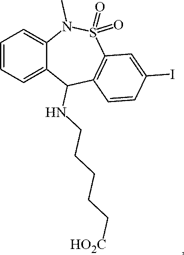
or a pharmaceutically acceptable salt or ester thereof.

11. The compound of claim 1 having the structure: 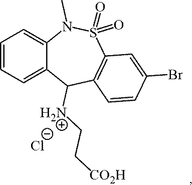 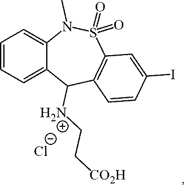 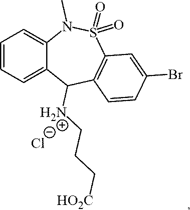 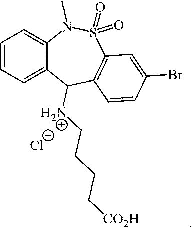 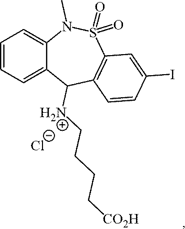 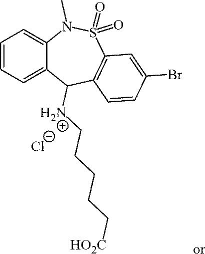 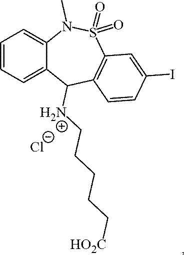
or an ester thereof.

12. The compound of claim 1 having the structure: 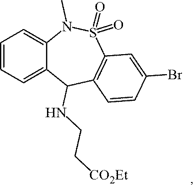 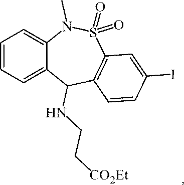 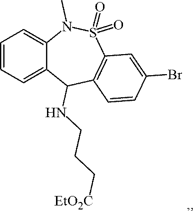 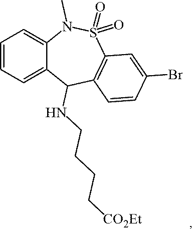 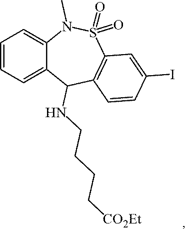 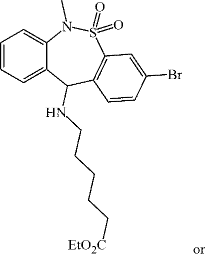 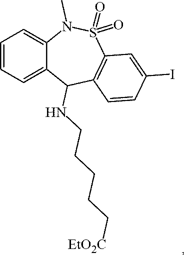
or a pharmaceutically acceptable salt or ester thereof.

13. A pharmaceutical composition comprising the compound of claim 1 and a pharmaceutically acceptable carrier.

14. (canceled)

15. A method of treating a neurological disorder in a subject, wherein the neurological disorder is selected from Huntington’s disease; Rett syndrome; a Rett syndrome variant; Angelman syndrome; Prader-Willi syndrome; neonatal onset encephalopathy; X-linked recessive mental retardation; fetal alcohol spectrum disorder; Hirschsprung disease; CDKL5 disorder; West syndrome; FOXG1 syndrome; or Lennox-Gastaut syndrome comprising administering an effective amount of the compound of claim 1 to the subject so as to treat the neurological disorder.

16. A method of treating Huntington’s disease, Rett syndrome, or CDKL5 disorder in a subject comprising administering an effective amount of the compound of claim 1 to the subject so as to treat the Huntington’s disease, Rett syndrome, or CDKL5 disorder.

17-31. (canceled)
